SEQUENCE LISTING (17 sequences)

SEQ ID NO: 1 - protein, 15 aa, Artificial Sequence
  Description of Artificial Sequence/note = Synthetic Construct
HAEDFARRRKRSVSL

SEQ ID NO: 2 - protein, 17 aa, Artificial Sequence
  Description of Artificial Sequence/note = Synthetic Construct
DKEASLGLWRTGSFLCK

SEQ ID NO: 3 - protein, 17 aa, Artificial Sequence
  Description of Artificial Sequence/note = Synthetic Construct
MDPEETSVYLDYYYATS

SEQ ID NO: 4 - protein, 15 aa, Artificial Sequence
  Description of Artificial Sequence/note = Synthetic Construct
SGLRQEHYLPSAILQ

SEQ ID NO: 5 - protein, 18 aa, Artificial Sequence
  Description of Artificial Sequence/note = Synthetic Construct
RELTLIDDKPYCAEKKAT

SEQ ID NO: 6 - protein, 18 aa, Artificial Sequence
  Description of Artificial Sequence/note = Synthetic Construct
KNYDFGSSTETSDSHLTK

SEQ ID NO: 7 - protein, 511 aa, Artificial Sequence
  Description of Artificial Sequence/note = Synthetic Construct
MRVKEKYQHLWRWGWRWGTMLLGMLMICSATEKLWVTVYYGVPVWKEATTTLFCASDAKAYDTEVHNVWATHACVPTDPNPQEVVLVNVTENFNMWKNDMVEQMHEDIISLWDQSLKPCVKLTPLCVSLKCTDLKNDTNTNSSSGRMIMEKGEIKNCSFNISTSIRGKVQKEYAFFYKLDIIPIDNDTTSYTLTSCNTSVITQACPKVSFEPIPIHYCAPAGFAILKCNNKTFNGTGPCTNVSTVQCTHGIRPVVSTQLLLNGSLAEEEVVIRSANFTDNAKTIIVQLNQSVEINCTRPNNNTRKSIRIQRGPGRAFVTIGKIGNMRQAHCNISRAKWNNTLKQIDSKLREQFGNNKTIIFKQSSGGDPEIVTHSFNCGGEFFYCNSTQLFNSTWFNSTWSTKGSNNTEGSDTITLPCRIKQIINMWQEVGKAMYAPPISGQIRCSSNITGLLLTRDGGNSNNESEIFRPGGGDMRDNWRSELYKYKVVKIEPLGVAPTKAKRRVVQREKR

SEQ ID NO: 8 - protein, 15 aa, Artificial Sequence
  Description of Artificial Sequence/note = Synthetic Construct
HAEDFARRRKRSISL

SEQ ID NO: 9 - protein, 360 aa, Artificial Sequence
  Description of Artificial Sequence/note = Synthetic Construct
MDPEETSVYLDYYYATSPNSDIRETHSHVPYTSVFLPVFYTAVFLTGVLGNLVLMGALHFKPGSRRLIDIFIINLAASDFIFLVTLPLWVDKEASLGLWRTGSFLCKGSSYMISVNMHCSVLLLTCMSVDRYLAIVWPVVSRKFRRTDCAYVVCASIWFISCLLGLPTLLSRELTLIDDKPYCAEKKATPIKLIWSLVALIFTFFVPLLSIVTCYCCIARKLCAHYQQSGKHNKKLKKSIKIIFIVVAAFLVSWLPFNTFKFLAIVSGLRQEHYLPSAILQLGMEVSGPLAFANSCVNPFIYYIFDSYIRRAIVHCLCPCLKNYDFGSSTETSDSHLTKALSTFIHAEDFARRRKRSVSL

SEQ ID NO: 10 - DNA, 1533 bp, Artificial Sequence
  Description of Artificial Sequence/note = Synthetic Construct
atgagagtgaaggagaaatatcagcacttgtggagatgggggtggagatggggcaccatgctccttgggatgttgatgatctgtagtgctacagaaaaattgtgggtcacagtctattatggggtacctgtgtggaaggaagcaaccaccactctattttgtgcatcagatgctaaagcatatgatacagaggtacataatgtttgggccacacatgcctgtgtacccacagaccccaacccacaagaagtagtattggtaaatgtgacagaaaattttaacatgtggaaaaatgacatggtagaacagatgcatgaggatataatcagtttatgggatcaaagcctaaagccatgtgtaaaattaaccccactctgtgttagtttaaagtgcactgatttgaagaatgatactaataccaatagtagtagcgggagaatgataatggagaaaggagagataaaaaactgctctttcaatatcagcacaagcataagaggtaaggtgcagaaagaatatgcatttttttataaacttgatataataccaatagataatgatactaccagctatacgttgacaagttgtaacacctcagtcattacacaggcctgtccaaaggtatcctttgagccaattcccatacattattgtgccccggctggttttgcgattctaaaatgtaataataagacgttcaatggaacaggaccatgtacaaatgtcagcacagtacaatgtacacatggaattaggccagtagtatcaactcaactgctgttaaatggcagtctggcagaagaagaggtagtaattagatctgccaatttcacagacaatgctaaaaccataatagtacagctgaaccaatctgtagaaattaattgtacaagacccaacaacaatacaagaaaaagtatccgtatccagagaggaccagggagagcatttgttacaataggaaaaataggaaatatgagacaagcacattgtaacattagtagagcaaaatggaataacactttaaaacagatagatagcaaattaagagaacaatttggaaataataaaacaataatctttaagcagtcctcaggaggggacccagaaattgtaacgcacagttttaattgtggaggggaatttttctactgtaattcaacacaactgtttaatagtacttggtttaatagtacttggagtactaaagggtcaaataacactgaaggaagtgacacaatcaccctcccatgcagaataaaacaaattataaacatgtggcaggaagtaggaaaagcaatgtatgcccctcccatcagtggacaaattagatgttcatcaaatattacagggctgctattaacaagagatggtggtaatagcaacaatgagtccgagatcttcagacctggaggaggagatatgagggacaattggagaagtgaattatataaatataaagtagtaaaaattgaaccattaggagtagcacccaccaaggcaaagagaagagtggtgcagagagaaaaaaga

SEQ ID NO: 11 - DNA, 1083 bp, Artificial Sequence
  Description of Artificial Sequence/note = Synthetic Construct
atggacccagaagaaacttcagtttatttggattattactatgctacgagcccaaactctgacatcagggagacccactcccatgttccttacacctctgtcttccttccagtcttttacacagctgtgttcctgactggagtgctggggaaccttgttctcatgggagcgttgcatttcaaacccggcagccgaagactgatcgacatctttatcatcaatctggctgcctctgacttcatttttcttgtcacattgcctctctgggtggataaagaagcatctctaggactgtggaggacgggctccttcctgtgcaaagggagctcctacatgatctccgtcaatatgcactgcagtgtcctcctgctcacttgcatgagtgttgaccgctacctggccattgtgtggccagtcgtatccaggaaattcagaaggacagactgtgcatatgtagtctgtgccagcatctggtttatctcctgcctgctggggttgcctactcttctgtccagggagctcacgctgattgatgataagccatactgtgcagagaaaaaggcaactccaattaaactcatatggtccctggtggccttaattttcaccttttttgtccctttgttgagcattgtgacctgctactgttgcattgcaaggaagctgtgtgcccattaccagcaatcaggaaagcacaacaaaaagctgaagaaatctataaagatcatctttattgtcgtggcagcctttcttgtctcctggctgcccttcaatactttcaagttcctggccattgtctctgggttgcggcaagaacactatttaccctcagctattcttcagcttggtatggaggtgagtggacccttggcatttgccaacagctgtgtcaaccctttcatttactatatcttcgacagctacatccgccgggccattgtccactgcttgtgcccttgcctgaaaaactatgactttgggagtagcactgagacatcagatagtcacctcactaaggctctctccaccttcattcatgcagaagattttgccaggaggaggaagaggtctgtgtcactctaa

SEQ ID NO: 12 - protein, 35 aa, Artificial Sequence
  Description of Artificial Sequence/note = Synthetic Construct
CTRPNNNTRRRLSIGPGRAFYARRNIIGDIRQAHC

SEQ ID NO: 13 - protein, 35 aa, Artificial Sequence
  Description of Artificial Sequence/note = Synthetic Construct
CTRPNNNTRKRISMGPGRVFYTTKDIVGDIRQAHC

SEQ ID NO: 14 - DNA, 9193 bp, Artificial Sequence
  Description of Artificial Sequence/note = Synthetic Construct
ggtctctcnngttagaccagatttgagcctggnagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggaggcgactggtgagtacgccaaaaattttgactagcggaggctagaaggagagagatgggtgcgagagcgtcagtattaagcgggggagaattagatcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaaaaaagcacagcaagcagcagctgacacaggacacagcagccaggtcagccaaaattaccctatagtgcagaacatccaggggcaaatggtacatcaggccatatcacctagaactttaaatgcatgggtaaaagtagtagaagagaaggctttcagcccagaagtgatacccatgttttcagcattatcagaaggagccaccccacaagatttaaacaccatgctaaacacagtggggggacatcaagcagccatgcaaatgttaaaagagaccatcaatgaggaagctgcagaatgggatagagtgcatccagtgcatgcagggcctattgcaccaggccagatgagagaaccaaggggaagtgacatagcaggaactactagtacccttcaggaacaaataggatggatgacaaataatccacctatcccagtaggagaaatttataaaagatggataatcctgggattaaataaaatagtaagaatgtatagccctaccagcattctggacataagacaaggaccaaaagaaccctttagagactatgtagaccggttctataaaactctaagagccgagcaagcttcacaggaggtaaaaaattggatgacagaaaccttgttggtccaaaatgcgaacccagattgtaagactattttaaaagcattgggaccagcagctacactagaagaaatgatgacagcatgtcagggagtgggaggacccggccataaggcaagagttttggctgaagcaatgagccaagtaacaaattcagctaccataatgatgcaaagaggcaattttaggaaccaaagaaagattgttaagtgtttcaattgtggcaaagaagggcacatagccagaaattgcagggcccctaggaaaaagggctgttggaaatgtggaaaggaaggacaccaaatgaaagattgtactgagagacaggctaattttttagggaagatctggccttcctacaagggaaggccagggaattttcttcagagcagaccagagccaacagccccaccagaagagagcttcaggtctggggtagagacaacaactccctctcagaagcaggagccgatagacaaggaactgtatcctttaacttccctcagatcactctttggcaacgacccctcgtcacaataaagataggggggcaactaaaggaagctctattagatacaggagcagatgatacagtattagaagaaatgagtttgccaggaagatggaaaccaaaaatgatagggggaattggaggttttatcaaagtaagacagtatgatcagatactcatagaaatctgtggacataaagctataggtacagtattagtaggacctacacctgtcaacataattggaagaaatctgttgactcagattggttgcactttaaattttcccattagtcctattgaaactgtaccagtaaaattaaagccaggaatggatggcccaaaagttaaacaatggccattgacagaagaaaaaataaaagcattagtagaaatttgtacagaaatggaaaaggaagggaaaatttcaaaaattgggcctgaaaatccatacaatactccagtatttgccataaagaaaaaagacagtactaaatggagaaaattagtagatttcagagaacttaataagagaactcaagacttctgggaagttcaattaggaataccacatcccgcagggttaaaaaagaaaaaatcagtaacagtactggatgtgggtgatgcatatttttcagttcccttagatgaagacttcaggaagtatactgcatttaccatacctagtataaacaatgagacaccagggattagatatcagtacaatgtgcttccacagggatggaaaggatcaccagcaatattccaaagtagcatgacaaaaatcttagagccttttagaaaacaaaatccagacatagttatctatcaatacatggatgatttgtatgtaggatctgacttagaaatagggcagcatagaacaaaaatagaggagctgagacaacatctgttgaggtggggacttaccacaccagacaaaaaacatcagaaagaacctccattcctttggatgggttatgaactccatcctgataaatggacagtacagcctatagtgctgccagaaaaagacagctggactgtcaatgacatacagaagttagtgggaaaattgaattgggcaagtcagatttacccagggattaaagtaaggcaattatgtaaactccttagaggaaccaaagcactaacagaagtaataccactaacagaagaagcagagctagaactggcagaaaacagagagattctaaaagaaccagtacatggagtgtattatgacccatcaaaagacttaatagcagaaatacagaagcaggggcaaggccaatggacatatcaaatttatcaagagccatttaaaaatctgaaaacaggaaaatatgcaagaacgaggggtgcccacactaatgatgtaaaacaattaacagaggcagtgcaaaaaataaccacagaaagcatagtaatatggggaaagactcctaaatttaaactacccatacaaaaggaaacatgggaaacatggtggacagagtattggcaagccacctggattcctgagtgggagtttgtcaatacccctcctttagtgaaattatggtaccagttagagaaagaacccatagtaggagcagaaacgttctatgtagatggggcagctagcagggagactaaattaggaaaagcaggatatgttactaatagaggaagacaaaaagttgtcaccctaactgacacaacaaatcagaagactgagttacaagcaattcatctagctttgcaggattcgggattagaagtaaatatagtaacagactcacaatatgcattaggaatcattcaagcacaaccagataaaagtgaatcagagttagtcaatcaaataatagagcagttaataaaaaaggaaaaggtctatctggcatgggtaccagcacacaaaggaattggaggaaatgaacaagtagataaattagtcagtgctggaatcaggaaagtactatttttagatggaatagataaggcccaagatgaacatgagaaatatcacagtaattggagagcaatggctagtgattttaacctgccacctgtagtagcaaaagaaatagtagccagctgtgataaatgtcagctaaaaggagaagccatgcatggacaagtagactgtagtccaggaatatggcaactagattgtacacatttagaaggaaaagttatcctggtagcagttcatgtagccagtggatatatagaagcagaagttattccagcagaaacagggcaggaaacagcatactttcttttaaaattagcaggaagatggccagtaaaaacaatacatacagacaatggcagcaatttcaccagtactacggttaaggccgcctgttggtgggcgggaatcaagcaggaatttggaattccctacaatccccaaagtcaaggagtagtagaatctatgaataaagaattaaagaaaattataggccaggtaagagatcaggctgaacatcttaagacagcagtacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccactttggaaaggaccagcaaagctcctctggaaaggtgaaggggcagtagtaatacaagataatagtgacataaaagtagtgccaagaagaaaagcaaagatcattagggattatggaaaacagatggcaggtgatgattgtgtggcaagtagacaggatgaggattagaacatggaaaagtttagtaaaacaccatatgtatgtttcagggaaagctaggggatggttttatagacatcactatgaaagccctcatccaagaataagttcagaagtacacatcccactaggggatgctagattggtaataacaacatattggggtctgcatacaggagaaagagactggcatctgggtcagggagtctccatagaatggaggaaaaagagatatagcacacaagtagaccctgaactagcagaccaactaattcatctgtattactttgactgtttttcagactctgctataagaaaggccttattaggacatatagttagccctaggtgtgaatatcaagcaggacataacaaggtaggatctctacaatacttggcactagcagcattaataacaccaaaaaagataaagccacctttgcctagtgttacgaaactgacagaggatagatggaacaagccccagaagaccaagggccacagagggagccacacaatgaatggacactagagcttttagaggagcttaagaatgaagctgttagacattttcctaggatttggctccatggcttagggcaacatatctatgaaacttatggggatacttgggcaggagtggaagccataataagaattctgcaacaactgctgtttatccatttcagaattgggtgtcgacatagcagaataggcgttactcaacagaggagagcaagaaatggagccagtagatcctagactagagccctggaagcatccaggaagtcagcctaaaactgcttgtaccacttgctattgtaaaaagtgttgctttcattgccaagtttgtttcacaacaaaagccttaggcatctcctatggcangaagaagcggagacagcgacgaanacctcctcaaggcagtcagactcatcaagtttctctatcaaagcagtaagtagtacatgtaatgcaacctatacaaatagcaatagcagcattagtagtagcaataataatagcaatagttgtgtggtccatagtaatcatagaatataggaaaatattaagacaaagaaaaatagacaggttaattgatagactaatagaaagagcagaagacagtggcaatgagagtgaaggagaaatatcagcacttgtggagatgggggtggaaatggggcaccatgctccttgggatattgatgatctgtagtgctacagaaaaattgtgggtcacagtctattatggggtacctgtgtggaaggaagcaaccaccactctattttgtgcatcagatgctaaagcatatgatacagaggtacataatgtttgggccacacatgcctgtgtacccacagaccccaacccacaagaagtagtattggtaaatgtgacagaaaattttaacatgtggaaaaatgacatggtagaacagatgcatgaggatataatcagtttatgggatcaaagcctaaagccatgtgtaaaattaaccccactctgtgttagtttaaagtgcactgatttggggaatgctactaataccaatagtagtaataccaatagtagtagcggggaaatgatgatggagaaaggagagataaaaaactgctctttcaatatcagcacaagcataagaggtaaggtgcagaaagaatatgcatttttttataaacttgatataataccaatagataatgatactaccagctatacgttgacaagttgtaacacctcagtcattacacaggcctgtccaaaggtatcctttgagccaattcccatacattattgtgccccggctggttttgcgattctaaaatgtaataataagacgttcaatggaacaggaccatgtacaaatgtcagcacagtacaatgtacacatggaattaggccagtagtatcaactcaactgctgttgaatggcagtctagcagaagaagaggtagtaattagatctgccaatttcacagacaatgctaaaaccataatagtacagctgaaccaatctgtagaaattaattgtacaagacccaacaacaatacaagaaaaagtatccgtatccagaggggaccagggagagcatttgttacaataggaaaaataggaaatatgagacaagcacattgtaacattagtagagcaaaatggaatgccactttaaaacagatagctagcaaattaagagaacaatttggaaataataaaacaataatctttaagcaatcctcaggaggggacccagaaattgtaacgcacagttttaattgtggaggggaatttttctactgtaattcaacacaactgtttaatagtacttggtttaatagtacttggagtactgaagggtcaaataacactgaaggaagtgacacaatcacactcccatgcagaataaaacaatttataaacatgtggcaggaagtaggaaaagcaatgtatgcccctcccatcagcggacaaattagatgttcatcaaatattacagggctgctattaacaagagatggtggtaataacaacaatgggtccgagatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcacggtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaataacatgacctggatggagtgggacagagaaattaacaattacacaagcttaatacattccttaattgaagaatcgcaaaaccagcaagaaaagaatgaacaagaattattggaattagataaatgggcaagtttgtggaattggtttaacataacaaattggctgtggtatataaaaatattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatccttagcacttatctgggacgatctgcggagcctgtgcctcttcagctaccaccgcttgagagacttactcttgattgtaacgaggattgtggaacttctgggacgcagggggtgggaagccctcaaatattggtggaatctcctacagtattggagtcaggaactaaagaatagtgctgttagcttgctcaatgccacagccatagcagtagctgaggggacagatagggttatagaagtagtacaaggagcttgtagagctattcgccacatacctagaagaataagacagggcttggaaaggattttgctataagatgggtggcaagtggtcaaaaagtagtgtggttggatggcctactgtaagggaaagaatgagacgagctgagccagcagcagatggggtgggagcagcatctcgagacctggaaaaacatggagcaatcacaagtagcaatacagcagctaccaatgctgcttgtgcctggctagaagcacaagaggaggaggaggtgggttttccagtcacacctcaggtacctttaagaccaatgacttacaaggcagctgtagatcttagccactttttaaaagaaaaggggggactggaagggctaattcactcccaacgaagacaagatatccttgatctgtggatctaccacacacaaggctacttccctgattggcagaactacacaccagggccaggggtcagatatccactgacctttggatggtgctacaagctagtaccagttgagccagataaggtagaagaggccaataaaggagagaacaccagcttgttacaccctgtgagcctgcatggaatggatgaccctgagagagaagtgttagagtggaggtttgacagccgcctagcatttcatcacgtggcccgagagctgcatccggagtacttcaagaactgctgacatcgagcttgctacaagggactttccgctggggactttccagggaggcgtggcctggnngggactggggagtggcgagccctcagatgctgcatataagcagctgctttttgcctgtactgggtctctctggttagaccagatttgagcctgggagctctctggctaactagggaacccactgcttaagcctcaataaagcttgccttgagtgcttca

SEQ ID NO: 15 - protein, 6 aa, Artificial Sequence
  Description of Artificial Sequence/note = Synthetic Construct
GPGRAF

SEQ ID NO: 16 - protein, 6 aa, Artificial Sequence
  Description of Artificial Sequence/note = Synthetic Construct
IQRGPG

SEQ ID NO: 17 - protein, 9 aa, Artificial Sequence
  Description of Artificial Sequence/note = Synthetic Construct
GPGRAFVTI

(19) United States
(12) Patent Application Publication
(10) Pub. No.: US 2005/0042747 A1
(43) Pub. Date: Feb. 24, 2005

(54) Hivgp120-induced bob/gpr15 activation

(76) Inventors: Frederic Clayton, Salt Lake City, UT (US); Jacques Fantini, Septemes-les-Vallons (FR)

Correspondence Address: NEEDLE & ROSENBERG, P.C., SUITE 1000, 999 PEACHTREE STREET, ATLANTA, GA, 30309-3915, US

(21) Appl. No.: 10/494,161

(22) PCT Filed: Oct. 25, 2002
(86) PCT No.: PCT/US02/34336

(30) Foreign Application Priority Data
Oct. 29, 2001 (US) 60341045

(51) Int. Cl. C12N 5/06; A61K 39/395; C12N 5/16; C12P 21/06

(52) U.S. Cl. 435/331; 435/344.1; 435/345; 424/143.1; 424/156.1

(57) ABSTRACT

Disclosed are compositions and methods for reducing the interaction between gp120 and Bob.

This application claims benefit of U.S. Provisional Application No. 60/341,045, filed Oct. 29, 2001, which is hereby incorporated herein by reference in its entirety.

### I. ACKNOWLEDGEMENTS

The research leading to the present invention was funded in part by Veterans Administration research funds. The government may have certain rights in the invention.

### H. BACKGROUND

Human immunodeficiency viruses (HIV) infect many differenant types of cells including macrophages, lymphocytes, and epithelial cells. HIV infection of these and other cells occurs through interactions between the HIV viral particle and the cell membrane. These interactions are mediated by receptors on the cell surface, within the cell membrane, and typically a coat protein on HIV call gp120. Different types of cells can be infected through different receptors or sets of receptors. Disclosed herein is an HIV co-receptor that can be found, for example, on intestinal epithelial cells. This co-receptor, which is the G-coupled protein Bob, also is shown herein to mediate the calcium induced activation of cells which can lead to many HIV effects, including increased infectivity and HIV enteropathy. Compositions that inhibit the interactions between Bob and gp120, methods of inhibiting HIV infection and effects, and methods of identifying compositions that modulate the Bob-gp120 interaction are disclosed, for example.

### III. SUMMARY

Disclosed are compositions and methods related to gp120 activation of lymphocytes, epithelial cells, and related cells.

### IV. BRIEF DESCRIPTION OF THE DRAWINGS

FIG. 1 shows tissue and cell line of Bob expression in Western blots. FIG. 1A shows Protein homogenates of 1) colonic mucosa, 2) small bowel mucosa, 3) lymph node, 4) testis, 5) lung, 6) brain, 7) placenta, 8) skeletal muscle, 9) ovary, 10) liver, 11) heart, 12) prostate, and 13) pancreas. FIG. 1B shows HT-29 antiBob immunoprecipitation. HT-29 eluates were immunoprecipitated with either Bob37 antibody (indicated by “B”) or polyclonal antigalactosyl ceramide antibody (indicated by “G”). Western blots were stained with either the Bob37 or Bob39 antibodies or monoclonal antigalactosyl ceramide as indicated below the band. Each lane had a strong band at 36 Id).

FIG. 2 shows—Cellular localization of Bob. In situ hybridization showed granular staining of rectal epithelium and most lamina propria lymphocytes (A). Original magnification x450. Immunofluorescent staining (with Bob37) of a small bowel villus tip, with finely granular membranous staining, much of which was at the basal surface and near the apical surface and, to a lesser degree, along the lateral sides (B). Original magnification x550. By confocal microscopy, most colonic epithelial cells had granular cytoplasmic staining (C). Original magnification x700. Bob-transfected Ghost (3) cells had finely granular, predominantly cytoplasmic staining when stained with antiBob37 (D). CXCR4-transfected Ghost (3) cells, stained and photographed like the Bob-transfected cells, had no staining (E). Original magnification for (D) and (E) x270.

FIG. 3 shows calcium signaling was induced by gp120 and blocked by pertussis toxin, U73122, and antiBob antibodies. Treatment with gp120_{IIIB} (50 pM in Locke's buffer) induced an immediate calcium signal (A). Similar signals (not shown) were seen with gp120_{CM235} (10 nM). No significant gp120_{IIIB}-induced calcium signaling was seen after pretreatment with pertussis toxin (500 ng/ml for 18 hours) (B) or U73122 (10 μM for 30 minutes) (C). U73343, an inactive analog of U73122, had no inhibitory effect (data not shown). AntiBob37 (100 μg/ml for 18 hours) also inhibited the gp120_{IIIB}-induced calcium signaling (D). These cells were then treated with SDF-1, causing a substantial calcium signal (E). Calcium signals were observed in Ghost (3) cell Bob cells with 15 fM gp120_{IIIB} (F). Original magnification x160.

FIG. 4 shows the Gp120 effects on microtubules were blocked by antiBob, pertussis toxin, and U73122. Anti-acetylated tubulin antibodies stained microtubules in many HT-29 cells (A). Staining was markedly reduced by a one hour treatment with gp120CM235 (B), particularly among clusters of several cells. The gp120-induced microtubule loss was inhibited by antiBob37 antibodies, 100 μg/ml for 18 hours prior to the gp120 treatment (C). Original magnification x460.

FIG. 5 shows the microtubule depolymerization induced by purified gp120 (IIIB isolate). Differentiated HT-29-D4 cells were either not exposed (a-c) or exposed (d-f) to purified gp120 (IIB isolate) for 1 hr. Microtubules were stained with an tubulin antibody followed by fluorescein-conjugated anti-mouse IgG. Actin filaments were stained with rhodamine-phalloidin. Confocal images were acquired with a Leica laser scanning microscope. A series of three confocal planes corresponding to the apical, medium and basal regions of the cells (from left to right respectively) is presented.

FIG. 6 shows intracellular lumina in HT-29-D4 cells exposed to HIV-1 or purified gp120 (IIIB isolate). Differentiated HT-29-D4 cells were either not exposed (a-d) or exposed (e-h) to HIV-1(IIIB) for 16 hr. The cells were stained with rhodamine-phalloidin and analyzed by confocal microscopy. Four confocal planes from the apical to the basal part of the cells are presented for each experimental condition. Lumina are indicated with arrows. The presence of lumina was also observed after a 1 hr incubation with purified gp120 (IIIB) (i-1).

FIG. 7 shows ultrastructure of HT-29-D4 cells exposed to HIV-1 or purified gp120 (IIB isolate). Differentiated HT-29-D4 cells were exposed to HIV-1(IIIB) for 16 hr (a-c) or to purified gp120(IIIB) for 1 hr (d). The cells were then fixed with glutaraldehyde and processed for transmission electron microscopy analysis. As a result of virus or gp120 exposure, the cells formed numerous intra- and inter-cellular lumina. The ultrastructure of control HT-29-D4 cells is shown in (e). As, apical space; d, desmosome; arrow and ial, intracellular lumen; iel, intercellular lumen; is, intercellular space; n, nucleus; tj, tight junction.

FIG. 8 shows strain-specific effects of gp120 on the intestinal barrier function. A—Differentiated HT-29-D4 cells cultured in Transwell chambers were either not exposed (squares) or exposed to gp120 purified from HIV-1(IIIB) (circles) or HIV-1(SEN) (triangles). Electrophysiological parameters were continuously recorded for 14 hr in electrophysiological medium and the relative value of TEER (corresponding to the TEER value at a given time divided by TEER at time 0) is shown. B—Dose-dependent effects of gp120 purified from HIV-1(IIIB) (circles) or HIV-1(SEN) (triangles) on TEER after 20 min of incubation. C—Initial increase in TEER expressed as relative TEER after 20 min of treatment with various gp120 or with neurotensin.

FIG. 9 shows lack of effect of purified gp120 on chloride secretion. Differentiated HT-29-D4 cells cultured in Transwell chambers were first treated with forskolin and then with either neurotensin or gp120 purified from HIV-1(IIIB) full circles, 89.6 (open circles), or SEN (open triangles). The electrophysiological recording of control, mock-treated cells is shown with full squares.

FIG. 10 shows partial protection of intestinal cells from gp120-induced toxicity. Differentiated HT-29-D4 cells cultured in Transwell chambers were exposed to gp120 purified from HIV-1(SEN) in the presence of either the indicated antibody or the synthetic soluble analog of GalCer CA52 (100 microg/ml). Anti-CXCR4, anti-GalCer and anti-GPR15/Bob (Bob37+Bob39) antibodies were used at 20 microg/ml, 1:400, and 1:100 dilutions, respectively. These concentrations corresponded to saturation binding of each antibody on HT-29-D4 cells as determined by quantitative cellular enzyme-linked assay. The results show the initial increase in TEER expressed as relative TEER after 20 min of treatment.

FIG. 11 shows galCer-gp120 interactions measured at the air-water interface. A monomolecular film of GalCer was prepared at the air-water interface and gp120 purified from either IIIB (full squares), SEN (open circles) or 89.6 (full circles) isolates was added in the aqueous subphase at a concentration of 5 nM. The kinetics of the surface pressure increase induced by the viral glycoproteins are shown.

FIG. 12 shows a schematic representation of GPR15/Bob. The sequence and proposed membrane topology of GPR15/Bob are shown. By analogy with similar receptors, the Cys residues in extracellular loops 1 and 2 are proposed to form a disulfide bond. Conserved regions in cytoplasmic loops characteristic of G-protein-coupled receptors are indicated by shading.

FIG. 13 shows a schematic of gp120 virotoxin and Bob receptor interactions.

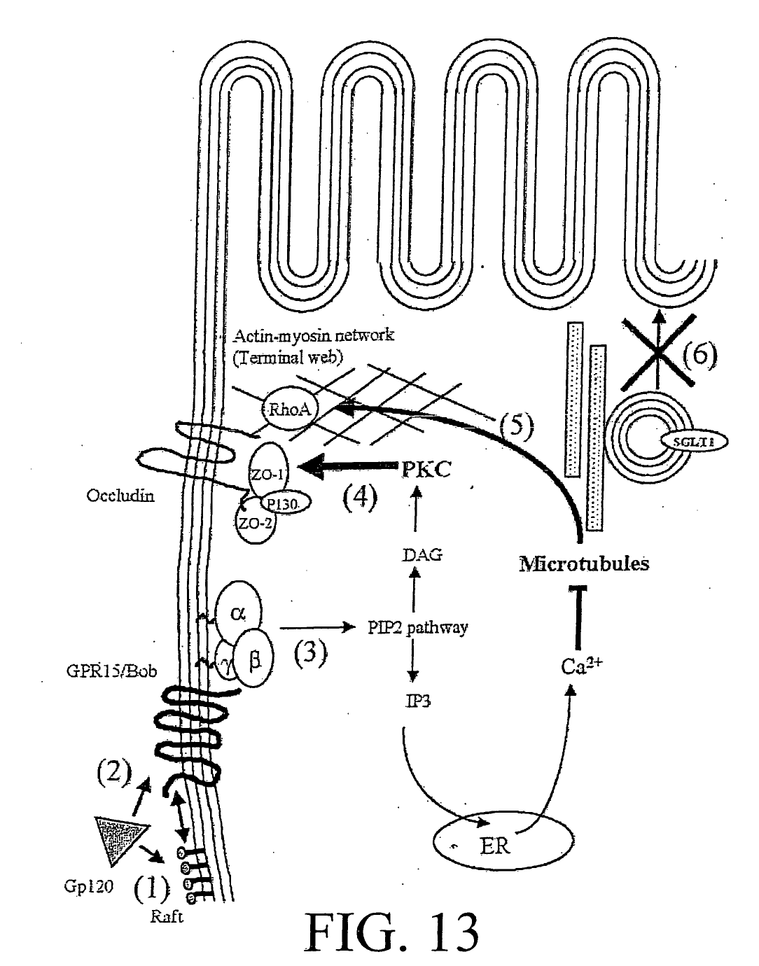

FIG. 14 shows peripheral blood mononuclear cells, loaded with the calcium sensitive fluorochrome Fluo-4. The left (darker) image is before the cells were treated, the right image was taken of the identical field 4 seconds after treatment with 50 picomolar gp120IIIB. Approximately 30% of the cells were substantially brighter after treatment with gp120, indicating an increase in intracellular calcium.

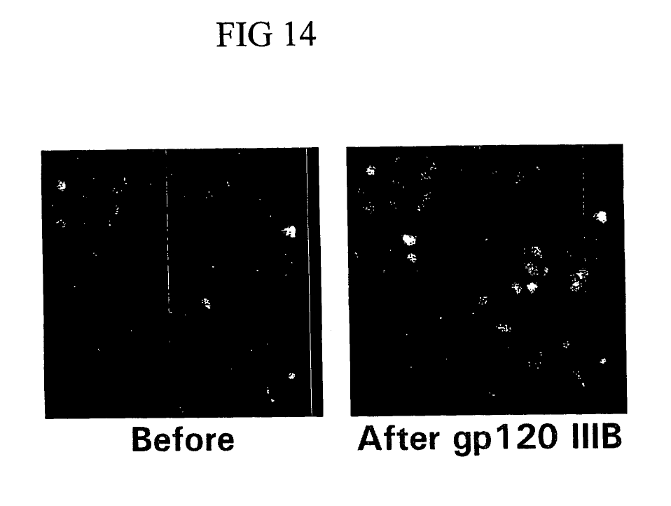

FIG. 15 shows HIV (strain 111B) infection of peripheral blood mononuclear cells, with or without overnight pretreatment with antiBob37 neutralizing antibody. Daily, from the 4^{th} day onward, ⅔ of the supernate was removed (and replaced). The removed supemate was assayed by ELISA for the content of HIV protein p24, with results expressed in nanograms per ml. The solid line and squares refer to the cells infected without the neutralizing antibody (an equivalent amount of nonspecific rabbit IgG was added instead). The dashed line and circles refers to the cells pretreated with antiBob37. Note that the neutralizing antibody pretreatment reduces the amount of virus produced by about 68%, and the peak of infection is delayed about one day.

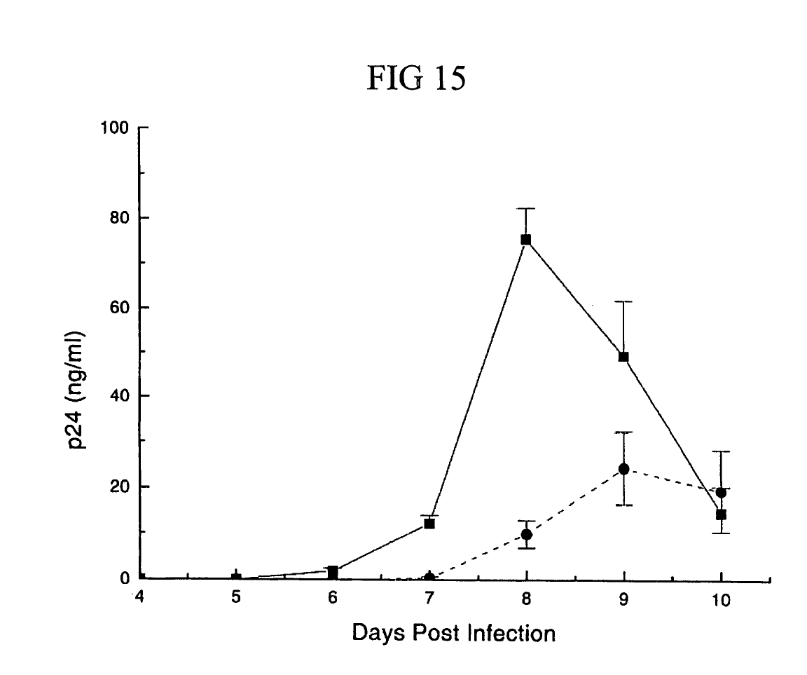

### IV. DETAILED DESCRIPTION

Human immunodeficiency viruses infect macrophages and activated CD4 lymphocytes. CD4 lymphocytes which are not activated are very resistant to infection. As HIV infection progresses, there is a decline in the number of CD4 lymphocytes and, simultaneously, such lymphocytes are found to have altered metabolism of inositol polyphosphates, intermediate signaling molecules that cause, among other things, calcium fluxes in the cells.

In vitro, normal peripheral blood lymphocytes are not easily HIV-1-infected unless stimulated, usually by phytohemaglutinin, which induces calcium signaling and renders them easily infected by appropriate strains of HIV. Calcium fluxes are known to cause actin based contraction. Actin based contraction induces the aggregation of surface molecules together on one side of the cell surface, a phenomenon called capping. Capping induces further activation, and is needed for full activation and an appropriate immune response. Effective HIV-1 infection only occurs when CD4 and the coreceptor (usually CCR5 or CXCR4) are clustered near each other, which capping promotes.

During asymptomatic HIV infection, there is a long period of stable or slowly declining CD4 lymphocyte content, then often a more rapid decline in the CD4 lymphocyte content that just precedes or coincides with the development of AIDS. It is plausible that this would coincide with the virus evolving the ability to activate resting CD4 lymphocytes, bringing them to the state of being easily HIV infected.

A further phenomenon that occurs in HIV infected subjects is CD4 lymphocyte apoptosis—a mechanism of cell death that contributes to the decline in CD4 lymphocytes that appears to cause AIDS. Calcium signaling is also known to promote apoptosis.

The inositol triphosphate signals are generated by phospholipases such as phospholipase C, which is itself activated by G proteins. G proteins are in turn activated by G protein coupled receptors, which include essentially all the HIV coreceptors. Prior studies had noted calcium fluxes and inositol phosphate signaling via CXCR4 and CCR5, caused by the HIV envelope protein gp120 of certain specific viral strains. However, these studies usually used nanomolar (10⁻⁹ M) concentrations of gp120, and the lowest concentration of gp120 which was effective was 0.2 nanomolar. However, these changes were seen in peripheral blood, where the amount of gp120 in most HIV infected subjects was subpicomolar (<10⁻¹² M). Thus there was scepticism that these receptors mediated this effect, and the mechanism by which physiologially relevant amounts of gp120 activates lymphocytes has been unclear.

A receptor causing calcium signaling, and thereby activating resting CD4 lymphocytes to a state in which they can be HIV-infected and in which they more easily undergo apoptosis or other cell death, could be present and would be relevant to progression from asymptomatic infection to AIDS. Inhibition of such a mechanism could delay or prevent progression to AIDS or help reverse the immunodeficiency.

Diarrhea, weight loss, and life-threatening cachexia are frequent problems in advanced HIV infection. Many HIV-infected subjects develop increased small intestinal permeability¹ and malabsorption of lipids² and sugars³ with minimal histologic findings.⁴ Since these occur without identifiable enteric infections, and improve with initial retroviral treatment,⁵ these problems were thought to be directly related to HIV and were thus termed HIV enteropathy. Gp120 induces calcium signaling and microtubule loss in the HT-29 intestinal cell line, resulting in malabsorption and increases in paracellular permeability resembling HIV enteropathy.^{6,7} Antibodies to the glycosphingolipid HIV receptor galactosyl ceramide caused similar changes. Evidence of reduced microtubule stability was also recently shown in the intestinal epithelium of HIV-infected subjects.⁸ Microtubule disrupting agents such as colchicine also cause malabsorption,9 increased intestinal permeability¹⁰, and diarrhea¹¹, similar to HIV enteropathy. It would be beneficial to known the cell signaling partners of gp120 caused calcium signaling and microtubule loss in intestinal epithelium.

The major HIV coreceptors CCRS and CXCR4 are both present in enteric epithelium as well as HT-29 cells, but in vivo they are present mainly at and near the luminal surface.¹² However, most productively HIV-infected cells in intestinal mucosa are superficial lamina propria macrophages.¹³ It was realized that a coreceptor present on the basolateral surface of the enteric epithelium would be a more plausible mediator of this effect, and disclosed herein is a coreceptor on the basolateral surface of enteric epithlium which interacts with gp120.

The orphan G-protein coupled receptor GPR15/Bob (hereafter called Bob).¹⁴⁻¹⁹ Although gp120 is the HIV-1 envelope surface protein, most of it is shed from infected cells rather than incorporated into virions.²⁰ It was thus realized that gp120 is a plausible mediator of toxic effects in uninfected cells. The principal HIV coreceptors (CXCR4 and CCR5) mediate gp120-induced calcium signaling, but prior studies demonstrated calcium signaling using these receptors only at relatively high gp120 concentrations (≧200 pM).²¹⁻²³ While the gp120 content probably varies widely in different tissue compartments, blood gp120 content in HIV-infected subjects is very low (0.075-0.80 pM), and is mostly in immune complexes.²⁴

Disclosed herein it is shown that 1) the Bob receptor is involved in gp120-induced signaling, 2) antibodies to Bob can block (neutralize) the gp120-induced signaling in cells that express Bob, 3) Bob mediates the gp120-induced effects seen in HT-29 cells, 4) Bob either interacts with galactosyl ceramide and/or cross reacts with antigalactosyl ceramide antibodies, and 5) Bob induces calcium signaling at the low gp120 concentrations anticipated in vivo.

The disclosed compounds, compositions, articles, devices, and/or methods are not limited to specific synthetic methods or specific recombinant biotechnology methods unless otherwise specified, or to particular reagents unless otherwise specified, as such may, of course, vary.

A. Definitions

As used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

Ranges may be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed.

“Optional” or “optionally” means that the subsequently described event or circumstance may or may not occur, and that the description includes instances where said event or circumstance occurs and instances where it does not.

“Primers” are a subset of probes which are capable of supporting some type of enzymatic manipulation and which can hybridize with a target nucleic acid such that the enzymatic manipulation can occur. A primer can be made from any combination of nucleotides or nucleotide derivatives or analogs available in the art, which do not interfere with the enzymatic manipulation.

“Probes” are molecules capable of interacting with a target nucleic acid, typically in a sequence specific manner, for example through hybridization. The hybridization of nucleic acids is well understood in the art and discussed herein. Typically a probe can be made from any combination of nucleotides or nucleotide derivatives or analogs available in the art.

Reducing means lowering or decreasing. It can be any amount of reduction, and would typically be determined as “reducing” by comparing amount of interest to a reference amount. For example, “reducing HIV enteropathy” would be any decrease of the HIV enteropathy symptoms in the presence of an inhibitor as compared to the amount of enteropathy symptoms in the absence of the inhibitor or a control. Any of the assays disclosed herein or known can be used to determine whether something is “reducing.”

Set of molecules. Disclosed are sets of molecules. These sets comprise at least two different molecules disclosed herein. The molecules can differ by as little as single atom up to being made up of completely different atoms. The sets can be made up of any of the disclosed compositions or combinations of compositions disclosed herein. A set of molecules can be referred to as a library of molecules.

A system as used herein refers to any combination of components that has a set of desired characteristics. For example, a system might be a cell that has been transfected with a particular gene or combination of genes so that the cell has certain properties. Or for example, the system could be a cell type that has been developed so as being capable of being used in HIV enteropathy analysis. For example, the Ghost 3 cells, expressing Bob disclosed herein can be considered a system. The system, could also be a, for example, chromatographic column having certain properties that would for example, include the covalent attachment of one of the disclosed peptides, such as SEQ ID Nos: 1-6.

Throughout this application, various publications are referenced. The disclosures of these publications in their entireties are hereby incorporated by reference into this application to support the disclosed compositions and methods. The references disclosed are also individually and specifically incorporated by reference herein for the material contained in them that is discussed in the sentence in which the reference is relied upon.

Disclosed are the components to be used to prepare the disclosed compositions as well as the compositions themselves and to be used within the methods disclosed herein. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a particular Bob molecule is disclosed and discussed and a number of modifications that can be made to a number of molecules including the Bob molecule are discussed, specifically contemplated is each and every combination and permutation of the Bob molecule and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods.

B. Compositions and Methods

Disclosed are compositions and methods that are drawn to gp120 induced activation of cells that are infected or can be infected by HIV. Disclosed is a mechanism by which gp120, by activating Bob (a G-protein coupled receptor), induces inositol triphosphate activation, calcium signaling, and thereby induce in lymphocytes a state of partial activation, rendering lymphocytes more easily HIV-infected or more susceptible to apoptosis. Agents reducing Bob activation by preventing gp120 activation are disclosed. Compositions and methods related to delaying or preventing productive HIV infection and thus the loss of CD4 lymphocytes and thus progression to immunodeficiency are disclosed. Also disclosed are compositions and methods for detecting Bob activation and calcium signaling by a given strain of HIV which can have bearing on the prognosis and on the appropriateness of anti-HIV therapy.

Disclosed are soluble Bob compositions and methods, such as inactivating substances, such as CA52 or other analogs of galactosyl ceramide or the modified amino acid citrulline, antibodies or other substances that bind ceramide or citrulline, or antibodies or other substances that bind the V3 region of gp120, or antibodies or other substances that bind Bob and thus block the gp120-induced activation in lymphocytes. Bob is highly conserved amongst individuals and therefore can act as a therapeutic target. Also disclosed are compositions and methods, such as inactivating substances, such as CA52, that inhibit Bob-mediated lymphocyte activation. Also disclosed are compositions and methods, such as CA52 that inhibit the loss of CD4 lymphocytes in HIV infected individuals.

Disclosed are compositions and methods for determining Bob activation and correlation of the same for diagnostic and prognostic purposes.

Cells typically must be activated to increase or enhance their susceptibility to HIV infection. The compositions and methods disclosed herein are drawn to the mechanisms and components involved in gp120 activation of cells when the gp120 concentrations are below about 0.15 nM.

The compositions and methods are drawn to the disclosure that Bob activation can occur through interactions with gp120 and that this activation causes calcium mediated HIV effects, and that the disclosed compositions and methods can mediate the gp120 and Bob interaction and thus mediate the calcium flux and thus mediate the HIV effects on cells.

1. Compositions

Disclosed are compositions that interact with Bob. Disclosed are compositions that interact with Bob such that Bob activation by HIV gp120 is reduced. It is disclosed herein that HIV gp120 interacts with Bob and that this interaction can activate Bob, causing calcium flux to occur in a cell in which Bob is associated. While any composition that inhibits activating gp120-Bob interactions is disclosed herein and these compositions can be identified using methods disclosed herein, there are different regions of Bob that can be specifically targeted by the compositions to reduce the gp120-Bob interaction. These regions are discussed herein.

a) Bob

Unless specifically indicated it is understood that Bob refers to any molecule considered a homolog of the protein set forth in SEQ ID NO:9. As discussed herein, there are allelic variants of the genes and homologs encoding Bob.

Unless specifically indicated, a substance that binds a region of Bob can be any type of molecule that interacts with Bob. For example, the substance could be a protein or a functional nucleic acid or a small molecule.

Reducing binding between Bob and gp120 means lowering or decreasing the binding relative to a control binding reaction. It can be any amount of reduction, and would typically be determined as “reducing” by comparing an amount of interest to a reference amount. For example, “reducing binding between Bob and gp120 would be any decrease of the Bob-gp120 binding in the presence of an inhibitor as compared to the amount of Bob-gp120 binding in the absence of the inhibitor or a control. Any of the assays disclosed herein or known can be used to determine whether the Bob-gp120 binding is reduced, including binding assays, cellular assays, activity assays, and infectivity assays.

The disclosed compositions can preferentially bind Bob rather than galactose-ceramide. Preferentially bind Bob over galactose ceramide means that the compositions bind Bob more tightly than they bind galactose ceramide. For example, the disclosed compositions, such as antibodies, bind Bob with Kds lower than the Kd with which they bind galactosyl ceramide. This could be of value because binding galactosyl ceramide or the various proteins which cross react with it (such as myelin basic protein) could potentially induce toxic effects. Antibodies to galactosly ceramide or other myelin components (myelin basic protein or MOG) could potentially induce a demyelinating neurologic process such as multiple sclerosis. Also disclosed compositions that bind Bob, but do not have detectable binding to neural tissue and specifically myelin. This includes the antiBob37 antibody, which is an effective Bob-neutralizing antibody but does not bind neural tissue as determined by Western blots as well as by immunofluorescent staining under cold conditions (0-5° C., conditions under which galactosyl ceramide and related glycosphyngolipids remain insoluble and could be stained. For certain compositions, when 5 micrograms per ml of a monoclonal antibody or 5 microcgrams per ml of an affinity purified polyclonal antibody are incubated in an immunostain of a tissue section experiment as described herein a band corresponding to galactosyl ceramide is not detectable beyond that present in a control reaction not containing the antibody.

Disclosed are compositions that reduce gp120 activation of cells, such as calcium activation. Gp120 activates cells by interaction with receptors on the cell surface. Activation of a G protein coupled receptor such as Bob causes activation of G proteins and phospholipase C and/or D activation, thus inducing an increase in inositol triphosphate and diacylglycerol content. Inositol triphosphate induces a calcium flux, and diacylglycerol induces activation of certain protein kinase C isoforms. Both calcium flux and treatment with diacylglycerol analogs (phorbol esters) enhance HIV proliferation in appropriate settings in vitro. Gp120-induced activation has been demonstrated in peripheral blood mononuclear cells such as lymphocytes and macrophages, cultured cells differentiating like intestinal epithelium, and Bob-transfected osteosarcoma cells. Comparable activation should occur in other Bob-expressing cell types such as renal tubular cells, other renal epithelial cells, germinal epithelium of the testis and sperm, prostatic epithelium, and hepatocytes. Activation has been detected by the detection of a calcium flux and, less directly, by a loss of microtubules and altered intestinal epithelial cell line differentiation and function. Disclosed herein blocking Bob activation retarded proliferation of HIV in vitro. Gp120 activation typically causes certain cell signaling events called gp120-induced cell signaling. As explained above, detection of calcium fluxes or protein kinase C activation provide relatively direct mechanisms to examine gp120-induced Bob activation. Disclosed are compositions that cause a requirement of at least 1.5, 2, 4, 6, 8, 10, 20, 30, 40, 50, 60, 70, 80, 90, 100, 150, 200, 300, 400, 500, 1000 fold more gp120 to be present in a calcium flux assay to get the same level of response as in a control assay run in the absence of the composition. Also disclosed are compositions that reduce amount of protein kinase C activation caused by HIV by at least 1, 5, 10, 15, 20, 25, 30, 40, 50 60, 70 80, 90, 91, 92, 93, 94, 95, 96, 97, 98, or 99%. Calcium flux and protein kinase C activation can be measured as discussed herein.

The disclosed compositions can delay and lessen the amount of productive infection of peripheral blood mononuclear cells, CD4 lymphocytes, and/or macrophages. By delay is meant an increase in the time that a cycle of productive HIV infection occurs in the presence of the disclosed compositions as compared to the absence of the disclosed compositions. This can be determined using the following assay. Peripheral blood mononulcear cells are grown in resting conditions, i.e. without serum, phytohemaglutinin, or other substances that could elicit a calcium flux. For at least 18 hours, some of the cells—as controls—are grown in the presence of a composition, such as Bob neutralizing antibody (such as antiBob37, 40-50 micrograms/ml). The cells are exposed to HIV (multiplicity of infection approximately 0.1 to 0.3) of a strain that activates Bob (such as HIV-1 LAI or HIV-1 IIIB) for 90-120 minutes in the presence of polybrene (1-2.5 micrograms/ml). The cells are then washed and the virus removed, and the cells are subsequently grown in RPMI 1640 supplimented with 10% fetal bovine serum, phytohemaglutinin-P (5 micrograms/ml), and IL-2 (25 U/ml). The supernates are sampled daily, starting with day four, and the supernates are examined by HIV p24 ELISA assay for the presence of productive viral infection. Disclosed are compositions that produce an increase in the amount of time for the effective HIV life cycle by at least 1, 2, 3, 4, 5, 6, 7, 10, 20, 30, 40, 50, 60, 70, 80, 90, 100, 200, 400, 600, 800, or 1,000%.

The carboxy terminal domain of Bob consists of amino acids from 301-311, such as 306 to the end of the protein of of SEQ ID NO:9, or the analogous positions of other sequences. (this determination is estimated from the appearance of a sequence of hydrophobic amino acids at about 305 onward toward the N terminal end.

The 1^{st} extracellular loop of Bob consists of approximately amino acids 86-96, such as 91, through 115-125, such as 120 of SEQ ID NO:9 or the analogous positions of other sequences (this determination is estimated from the appearance of a sequence of hydrophobic amino acids at about 90 and about 121).

The 2^{nd} extracellular loop of Bob consists of approximately amino acids 166-176, such as 171, through 187-197, such as 192 of SEQ ID NO:9 or the analogous position of other sequences (this determination is estimated from the appearance of a sequence of hydrophobic amino acids at about 170 and about 193).

The 3^{rd} extracellular loop of Bob consists of approximately amino acids 256-266 such as 261, through 279-289,such as 284 of SEQ ID NO:9 or the analogous position of other sequences (this determination is estimated from the appearance of a sequence of hydrophobic amino acids at about 260 and about 285.

The N-terminal extracellular domain of Bob is from amino acid 1 to approximately amino acid 28-38, such as 33 of SEQ ID NO:9 or the analogous position of other sequences (this determination is estimated from the appearance of a sequence of hydrophobic amino acids at about amino acid 34).

(1) Bob Expression Patterns

Bob is expressed in a number of tissues. Western Blot analysis can determine where Bob is expressed. Western Blot analysis has indicated that Bob is expressed in for example, in the lymph node, spleen, tonsil, peripheral blood mononuclear cells, colonic mucosa, small bowel mucosa, prostate, kidney, testis, and liver. Western Blot analysis has also indicated that certain tissues contain little or no Bob. For example, brain (grey matter and white matter), lung, pancreas, heart, skeletal muscle, ovary, fat, placenta, and uterine wall.

There are certain cell lines that also contain abundant Bob as determined by Western Blot. For example, intestinal cell lines: HT-29, HCT-116, HCA-7, the renal cell line: Madin-Darby canine kidney, and the lymphocytic Cell Line: Daudi. There are also specific cell types containing Bob as determined by immunostaining: colonic epithelium, small bowel epithelium, renal tubular epithelium, germinal epithelium of the testis, CD4-positive T cell lymphocytes, CD8-positive T cell lymphocytes, macrophages, CD20-positive B cell lymphocytes, and hepatocytes (in and near bile canaliculi).

(2) Bob Target Regions

Disclosed herein are certain regions that have been shown that if they are interacted with binding between Bob and gp120 can be reduced. These disclosed regions can be represented by the primary amino acid structure. It is understood, however, that these regions of amino acids form specific three dimensional structures and that recognition occurs at the three dimensional structural level. It is further understood that as these specific regions have been shown to be able to generate molecules that recognize them and that molecules that recognize them as a region also recognize the full length Bob polypeptide as well as the native peptide of Bob, these regions are capable of folding in three dimensional structures as regions that are similar to the structure these regions adopt in the full length Bob polypeptide. Six specific regions have been identified, as well as sets of these regions. One region comprises the sequence HAEDFARRRKRSVSL (SEQ ID NO:1). Another region comprises the sequence DKEASLGLWRTGSFLCK (SEQ ID NO:2). Another region comprises the sequence MDPEETSVYLDYYYATS (SEQ ID NO:3). Another region comprises the sequence SGLRQEHYLPSAILQ (SEQ ID NO:4). Another region comprises the sequence RELTLIDDKPYCAEKKAT (SEQ ID NO:5). Another region comprises the sequence KNYDFGSSTETSDSHLTK (SEQ ID NO:6).

SEQ ID NO:1 is a portion of the carboxy end of the carboxy terminal intracellular domain. SEQ ID NO:2 is the N terminal end of the first extracellular loop. SEQ ID NO:3 is the N terminal end of the extracellular domain. SEQ ID NO:4 is from the third extracellular loop and SEQ ID NO:5 is from the second extracellular loop. SEQ ID NO:6 is from the carboxy terminal intracellular domain.

Disclosed are compositions that inhibit gp120-Bob interactions, such as compositions that bind SEQ ID NO:2 and SEQ ID NO:3.

The antibodies raised towards SEQ ID NOs:1-6 had unusually high affinity for the peptides to SEQ ID NOs:2-6 and lower affinity towards SEQ ID NO:1. SEQ ID NO:1 can be found in the carboxy terminal and intracellular domain. The approximate minimum detection limits for the antibodies in the Bob39 preparation were detectable at <2.8*10⁻¹¹ M for SEQ IN NO:4, <2.8*10⁻¹¹ M for SEQ ID NO:5, and <2.8*10⁻¹¹ M for SEQ ID NO:6 and the antibodies for the Bob37 preparation were detectable at 1.4*10⁻⁷ M for SEQ ID NO:1, 2.8*10⁻¹¹ M for SEQ ID NO:2, and 2.8*10⁻¹¹ M for SEQ ID NO:3 in an Elisa assay (for example, solid phase of peptide (antigen), add dilutions of antibody (such as Bob37), add second antibody (recognizes IGG) with peroxydase, and then assay for color change.)

Disclosed are compositions that bind Bob with Kds of less than or equal to 10⁻⁵, 10⁻⁶ 10⁻⁷, 10⁻⁸, 10⁻⁹, 10⁻¹⁰, 10⁻¹¹, or 10⁻¹². Compositions can bind Bob with specificity. Disclosed are compositions wherein the compositions bind Bob with a Kd at least 5, 10, 25, 50, 75, 100, 125, 150, 200, 500, 1000, 5000, 10,000, 50,000, 100,000, 500,000, 10⁶, 10⁷, or 10⁸, fold lower than the Kd with binding a galactose ceramide or some other background molecule.

While compositions that bind any of the disclosed regions of Bob are capable of interfering with the gp120-Bob interaction, certain regions, not only elicit binding responses that bind Bob, but which are also highly specific for Bob, showing very little background binding to other proteins. When assessing a polyclonal sera raised to multiple antigens, binding specificity and indications can be obtained by incubating the target molecules with the antibody as well as an excess of one of the antigenic peptides used to generate the binding polyclonal sera. For example, a 100 to 500 fold excess of one of the peptides can be added and overnight incubation at 4° C. can be performed before being tested in immunostaining procedures. For example, assessing the Bob37 sera indicates the following, preincubation with SEQ ID NO:2, essentially eliminate nonspecific staining, while very strong specific staining remained; preincubation with SEQ ID NO:1, reduced the specific staining, although the nonspecific staining remained, and preincubation with SEQ ID NO:3, had no effect on the immunostaining pattern.

Assessing the Bob39 sera indicates the following, preincubation with SEQ ID NO:6, gave a strong and partially specific staining pattern, while adding either of the others reduced the specific staining. There is still cross staining with neural tissue proteins of about 90 kD. However, even with this absorption the nonspecific staining of neural tissue, particularly axons, remained.

Thus, the carboxy terminal end sequence defined at least in part by the sequence set forth in SEQ ID NO:1 represents one specific target for isolating compositions that bind specifically with Bob such that interactions with gp120 is reduced. The sequences set forth in SEQ ID NOs:5 and 6 which represent at least part of the 2^{nd} and 3^{rd} extracellular loop domains) and can also be used. SEQ ID NOs: 2, 3, and 4 can also be used to develop compositions that interact with Bob such that gp120-Bob interactions are reduced.

As a Western blot control test, all three appropriate peptides were added (to which an antibody was raised and affinity purified) and, after an overnight incubation, tested in Western blots. The 36 kD band corresponding to Bob was greatly reduced by the absorbing peptides.

The galactosyl ceramide artificial membrane results indicate that each of the three extracellular loop regions bind galactosyl ceramide, although the extracellular N terminal end domain and both the carboxy terminal domain peptides do not specifically bind galactosyl ceramide.

In neutralizing antibody tests, the Bob37 antibody was found to be much more effective than Bob39. Since the antibody would not have access to the intracellular carboxy terminal domain, this implies that the N terminal domain (MDPEET . . . ) or the first extracellular loop (DKEAS . . . ) or both must be accessible for the gp120-induced activation to occur.

b) Compositions that Interact with Bob, gp120, or Inhibit HIV

The disclosed compositions can be any composition that binds Bob such that a Bob-gp120 interacation is reduced. There are many differenant types of compositions that can perform this task, such as antibodies, peptides, peptide memetics, functional nucleic acids, such as aptamers, and small molecules. It is understood that molecules of each of these categories can be identified methods disclosed herein and that which is known, along with the disclosed information that Bob interacts with gp120, and information that certain regions of Bob can be involed in that interaction.

Disclosed are cells containing the disclosed compositions. The cells can be any type of cell. For example, the cell comprising the disclosed compositions can be lymphocyte, macrophage, intestinal epithelial cell, renal tubular or glomerular epithelial cell, hepatocytes, prostatic epithelial cells, and germinal epithelium of the testis.

Disclosed are animals comprising the disclosed compositions and cells comprising the disclosed compositions. It is understood that the disclosed animals can be any animal, including mammals, such as murines, mouse, rat, rabbit, hamster, ovines, such as sheep, bovines, such as a cow, equines, such as a horse, porcines, such as a pig, or primates, such as human, monkey, baboon, orangatang, goriila, or chimpanzee.

(1) Antibodies

As used herein, the term “antibody” encompasses, but is not limited to, whole immunoglobulin (i.e., an intact antibody) of any class. Native antibodies are usually heterotetrameric glycoproteins, composed of two identical light (L) chains and two identical heavy (H) chains. Typically, each light chain is linked to a heavy chain by one covalent disulfide bond, while the number of disulfide linkages varies between the heavy chains of different immunoglobulin isotypes. Each heavy and light chain also has regularly spaced intrachain disulfide bridges. Each heavy chain has at one end a variable domain (V(H)) followed by a number of constant domains. Each light chain has a variable domain at one end (V(L)) and a constant domain at its other end; the constant domain of the light chain is aligned with the first constant domain of the heavy chain, and the light chain variable domain is aligned with the variable domain of the heavy chain. Particular amino acid residues are believed to form an interface between the light and heavy chain variable domains. The light chains of antibodies from any vertebrate species can be assigned to one of two clearly distinct types, called kappa (k) and lambda (l), based on the amino acid sequences of their constant domains. Depending on the amino acid sequence of the constant domain of their heavy chains, immunoglobulins can be assigned to different classes. There are five major classes of human immunoglobulins: IgA, IgD, IgE, IgG and IgM, and several of these may be further divided into subclasses (isotypes), e.g., IgG-1, IgG-2, IgG-3, and IgG-4; IgA-1 and IgA-2. One skilled in the art would recognize the comparable classes for mouse. The heavy chain constant domains that correspond to the different classes of immunoglobulins are called alpha, delta, epsilon, gamma, and mu, respectively. The term antibody can encompass both polyclonal antibodies and monoclonal antibodies. It is understood that an antigenic reaction producing an antibody produces both polyclonal sera and that individual monoclonal antibodies can be isolated using standard procedures.

The term “variable” is used herein to describe certain portions of the variable domains that differ in sequence among antibodies and are used in the binding and specificity of each particular antibody for its particular antigen. However, the variability is not usually evenly distributed through the variable domains of antibodies. It is typically concentrated in three segments called complementarity determining regions (CDRs) or hypervariable regions both in the light chain and the heavy chain variable domains. The more highly conserved portions of the variable domains are called the framework (FR). The variable domains of native heavy and light chains each comprise four FR regions, largely adopting a b-sheet configuration, connected by three CDRs, which form loops connecting, and in some cases forming part of, the b-sheet structure. The CDRs in each chain are held together in close proximity by the FR regions and, with the CDRs from the other chain, contribute to the formation of the antigen binding site of antibodies (see Kabat E. A. et al., “Sequences of Proteins of Immunological Interest,” National Institutes of Health, Bethesda, Md. (1987)). The constant domains are not involved directly in binding an antibody to an antigen, but exhibit various effector functions, such as participation of the antibody in antibody-dependent cellular toxicity.

As used herein, the term “antibody or fragments thereof” encompasses chimeric antibodies and hybrid antibodies, with dual or multiple antigen or epitope specificities, and fragments, such as F(ab′)₂, Fab′, Fab and the like, including hybrid fragments, as well as antibodies. Thus, fragments of the antibodies that retain the ability to bind their specific antigens are provided. For example, fragments of antibodies which maintain Bob antigen binding activity are included within the meaning of the term “antibody or fragment thereof.” Such antibodies and fragments can be made by techniques known in the art and can be screened for specificity and activity according to general methods for producing antibodies and screening antibodies for specificity and activity (See Harlow and Lane. Antibodies, A Laboratory Manual. Cold Spring Harbor Publications, New York, (1988)).

Also included within the meaning of “antibody or fragments thereof” are conjugates of antibody fragments and antigen binding proteins (single chain antibodies) as described, for example, in U.S. Pat. No. 4,704,692, the contents of which are hereby incorporated by reference.

Optionally, the antibodies are generated in other species and “humanized” for administration in humans. Humanized forms of non-human (e.g., murine) antibodies are chimeric immunoglobulins, immunoglobulin chains or fragments thereof (such as Fv, Fab, Fab′, F(ab′)₂, or other antigen-binding subsequences of antibodies) which contain minimal sequence derived from non-human immunoglobulin. Humanized antibodies include human immunoglobulins (recipient antibody) in which residues from a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues that are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region (Fc), typically that of a human immunoglobulin (Jones et al., Nature, 321:522-525 (1986); Riechmann et al., Nature, 332:323-327 (1988); and Presta, Curr. Op. Struct. Biol., 2:593-596 (1992)).

Methods for humanizing non-human antibodies are well known in the art. Generally, a humanized antibody has one or more amino acid residues introduced into it from a source that is non-human. These non-human amino acid residues are often referred to as “import” residues, which are typically taken from an “import” variable domain. Humanization can be essentially performed following the method of Winter and co-workers (Jones et al., Nature, 321:522-525 (1986); Riechmann et al., Nature, 332:323-327 (1988); Verhoeyen et al., Science, 239:1534-1536 (1988)), by substituting rodent CDRs or CDR sequences for the corresponding sequences of a human antibody. Accordingly, such “humanized” antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

The choice of human variable domains, both light and heavy, to be used in making the humanized antibodies is very important in order to reduce antigenicity. According to the “best-fit” method, the sequence of the variable domain of a rodent antibody is screened against the entire library of known human variable domain sequences. The human sequence which is closest to that of the rodent is then accepted as the human framework (FR) for the humanized antibody (Sims et al., J. Immunol., 151:2296 (1993) and Chothia et al., J. Mol. Biol., 196:901 (1987)). Another method uses a particular framework derived from the consensus sequence of all human antibodies of a particular subgroup of light or heavy chains. The same framework may be used for several different humanized antibodies (Carter et al., Proc. Natl. Acad. Sci. USA, 89:4285 (1992); Presta et al., J. Immunol., 151:2623 (1993)).

It is further important that antibodies be humanized with retention of high affinity for the antigen and other favorable biological properties. To achieve this goal, according to a preferred method, humanized antibodies are prepared by a process of analysis of the parental sequences and various conceptual humanized products using three dimensional models of the parental and humanized sequences. Three dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, FR residues can be selected and combined from the consensus and import sequence so that the desired antibody characteristic, such as increased affinity for the target antigen(s), is achieved. In general, the CDR residues are directly and most substantially involved in influencing antigen binding (see, WO 94/04679, published 3 Mar. 1994).

Transgenic animals (e.g., mice) that are capable, upon immunization, of producing a full repertoire of human antibodies in the absence of endogenous immunoglobulin production can be employed. For example, it has been described that the homozygous deletion of the antibody heavy chain joining region (J(H)) gene in chimeric and germ-line mutant mice results in complete inhibition of endogenous antibody production. Transfer of the human germ-line immunoglobulin gene array in such germ-line mutant mice will result in the production of human antibodies upon antigen challenge (see, e.g., Jakobovits et al., Proc. Natl. Acad. Sci. USA, 90:2551-255 (1993); Jakobovits et al., Nature, 362:255-258 (1993); Bruggemann et al., Year in Immuno., 7:33 (1993)). Human antibodies can also be produced in phage display libraries (Hoogenboom et al., J. Mol. Biol., 227:381 (1991); Marks et al., J. Mol. Biol., 222:581 (1991)). The techniques of Cote et al. and Boerner et al. are also available for the preparation of human monoclonal antibodies (Cole et al., Monoclonal Antibodies and Cancer Therapy, Alan R. Liss, p. 77 (1985); Boemer et al., J. Immunol., 147(1):86-95 (1991)).

Disclosed are hybidoma cells that can produce monoclonal antibodies. The term “monoclonal antibody” as used herein refers to an antibody obtained from a substantially homogeneous population of antibodies, i.e., the individual antibodies comprising the population are identical except for possible naturally occurring mutations that may be present in minor amounts. The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired activity (See, U.S. Pat. No. 4,816,567 and Morrison et al., Proc. Natl. Acad. Sci. USA, 81:6851-6855 (1984)).

Monoclonal antibodies can be prepared using hybridoma methods, such as those described by Kohler and Milstein, Nature, 256:495 (1975) or Harlow and Lane. Antibodies, A Laboratory Manual. Cold Spring Harbor Publications, New York, (1988). In a hybridoma method, a mouse or other appropriate host animal, is typically immunized with an immunizing agent to elicit lymphocytes that produce or are capable of producing antibodies that will specifically bind to the immunizing agent. Alternatively, the lymphocytes may be immunized in vitro. Preferably, the immunizing agent comprises Bob. Traditionally, the generation of monoclonal antibodies has depended on the availability of purified protein or peptides for use as the immunogen. More recently DNA based immunizations have shown promise as a way to elicit strong immune responses and generate monoclonal antibodies. In this approach, DNA-based immunization can be used, wherein DNA encoding a portion of Bob expressed as a fusion protein with human IgG1 is injected into the host animal according to methods known in the art (e.g., Kilpatrick K E, et al. Gene gun delivered DNA-based immunizations mediate rapid production of murine monoclonal antibodies to the Flt-3 receptor. Hybridoma. 1998 December;17(6):569-76; Kilpatrick KE et al. High-affinity monoclonal antibodies to PED/PEA-15 generated using 5 microg of DNA. Hybridoma. 2000 August;19(4):297-302, which are incorporated herein by referenced in full for the the methods of antibody production).

An alternate approach to immunizations with either purified protein or DNA is to use antigen expressed in baculovirus. The advantages to this system include ease of generation, high levels of expression, and post-translational modifications that are highly similar to those seen in mammalian systems. Use of this system involves expressing domains of Bob antibody as fusion proteins. The antigen is produced by inserting a gene fragment in-frame between the signal sequence and the mature protein domain of the Bob antibody nucleotide sequence. This results in the display of the foreign proteins on the surface of the virion. This method allows immunization with whole virus, eliminating the need for purification of target antigens.

Generally, either peripheral blood lymphocytes (“PBLs”) are used in methods of producing monoclonal antibodies if cells of human origin are desired, or spleen cells or lymph node cells are used if non-human mammalian sources are desired. The lymphocytes are then fused with an immortalized cell line using a suitable fusing agent, such as polyethylene glycol, to form a hybridoma cell (Goding, “Monoclonal Antibodies: Principles and Practice” Academic Press, (1986) pp. 59-103). Immortalized cell lines are usually transformed mammalian cells, including myeloma cells of rodent, bovine, equine, and human origin. Usually, rat or mouse myeloma cell lines are employed. The hybridoma cells may be cultured in a suitable culture medium that preferably contains one or more substances that inhibit the growth or survival of the unfused, immortalized cells. For example, if the parental cells lack the enzyme hypoxanthine guanine phosphoribosyl transferase (HGPRT or HPRT), the culture medium for the hybridomas typically will include hypoxanthine, aminopterin, and thymidine (“HAT medium”), which substances prevent the growth of HGPRT-deficient cells. Preferred immortalized cell lines are those that fuse efficiently, support stable high level expression of antibody by the selected antibody-producing cells, and are sensitive to a medium such as HAT medium. More preferred immortalized cell lines are murine myeloma lines, which can be obtained, for instance, from the Salk Institute Cell Distribution Center, San Diego, Calif. and the American Type Culture Collection, Rockville, Md. Human myeloma and mouse-human heteromyeloma cell lines also have been described for the production of human monoclonal antibodies (Kozbor, J. Immunol., 133:3001 (1984); Brodeur et al., “Monoclonal Antibody Production Techniques and Applications” Marcel Dekker, Inc., New York, (1987) pp. 51-63). The culture medium in which the hybridoma cells are cultured can then be assayed for the presence of monoclonal antibodies directed against Bob. Preferably, the binding specificity of monoclonal antibodies produced by the hybridoma cells is determined by immunoprecipitation or by an in vitro binding assay, such as radioimmunoassay (RIA) or enzyme-linked immunoabsorbent assay (ELISA). Such techniques and assays are known in the art, and are described in Harlow and Lane “Antibodies, A Laboratory Manual” Cold Spring Harbor Publications, New York, (1988).

After the desired hybridoma cells are identified, the clones may be subcloned by limiting dilution or FACS sorting procedures and grown by standard methods. Suitable culture media for this purpose include, for example, Dulbecco's Modified Eagle's Medium and RPMI-1640 medium. Alternatively, the hybridoma cells may be grown in vivo as ascites in a mammal.

The monoclonal antibodies secreted by the subclones may be isolated or purified from the culture medium or ascites fluid by conventional immunoglobulin purification procedures such as, for example, protein A-Sepharose, protein G, hydroxylapatite chromatography, gel electrophoresis, dialysis, or affinity chromatography.

The monoclonal antibodies may also be made by recombinant DNA methods, such as those described in U.S. Pat. No. 4,816,567. DNA encoding the monoclonal antibodies can be readily isolated and sequenced using conventional procedures (e.g., by using oligonucleotide probes that are capable of binding specifically to genes encoding the heavy and light chains of murine antibodies). The hybridoma cells serve as a preferred source of such DNA. Once isolated, the DNA may be placed into expression vectors, which are then transfected into host cells such as simian COS cells, Chinese hamster ovary (CHO) cells, plasmacytoma cells, or myeloma cells that do not otherwise produce immunoglobulin protein, to obtain the synthesis of monoclonal antibodies in the recombinant host cells. The DNA also may be modified, for example, by substituting the coding sequence for human heavy and light chain constant domains in place of the homologous murine sequences (U.S. Pat. No. 4,816,567) or by covalently joining to the immunoglobulin coding sequence all or part of the coding sequence for a non-immunoglobulin polypeptide. Optionally, such a non-immunoglobulin polypeptide is substituted for the constant domains of an antibody or substituted for the variable domains of one antigen-combining site of an antibody to create a chimeric bivalent antibody comprising one antigen-combining site having specificity for Bob and another antigen-combining site having specificity for a different antigen.

In vitro methods are also suitable for preparing monovalent antibodies. Digestion of antibodies to produce fragments thereof, particularly, Fab fragments, can be accomplished using routine techniques known in the art. For instance, digestion can be performed using papain. Examples of papain digestion are described in WO 94/29348 published Dec. 22, 1994, U.S. Pat. No. 4,342,566, and Harlow and Lane, Antibodies, A Laboratory Manual, Cold Spring Harbor Publications, New York, (1988). Papain digestion of antibodies typically produces two identical antigen binding fragments, called Fab fragments, each with a single antigen binding site, and a residual Fc fragment. Pepsin treatment yields a fragment, called the F(ab′)2 fragment, that has two antigen combining sites and is still capable of cross-linking antigen.

The Fab fragments produced in the antibody digestion also contain the constant domains of the light chain and the first constant domain of the heavy chain. Fab′ fragments differ from Fab fragments by the addition of a few residues at the carboxy terminus of the heavy chain domain including one or more cysteines from the antibody hinge region. The F(ab′)2 fragment is a bivalent fragment comprising two Fab′ fragments linked by a disulfide bridge at the hinge region. Fab′-SH is the designation herein for Fab′ in which the cysteine residue(s) of the constant domains bear a free thiol group. Antibody fragments originally were produced as pairs of Fab′ fragments which have hinge cysteines between them. Other chemical couplings of antibody fragments are also known.

An isolated immunogenically specific paratope or fragment of the antibody is also provided. A specific immunogenic epitope of the antibody can be isolated from the whole antibody by chemical or mechanical disruption of the molecule. The purified fragments thus obtained are tested to determine their immunogenicity and specificity by the methods taught herein. Immunoreactive paratopes of the antibody, optionally, are synthesized directly. An immunoreactive fragment is defined as an amino acid sequence of at least about two to five consecutive amino acids derived from the antibody amino acid sequence.

One method of producing proteins comprising the disclosed antibodies is to link two or more peptides or polypeptides together by protein chemistry techniques. For example, peptides or polypeptides can be chemically synthesized using currently available laboratory equipment using either Fmoc (9-fluorenylmethyloxycarbonyl) or Boc (tert-butyloxycarbonoyl) chemistry. (Applied Biosystems, Inc., Foster City, Calif.). The disclosed peptides or polypeptides corresponding to the antibodies, for example, can be synthesized by standard chemical reactions. For example, a peptide or polypeptide can be synthesized and not cleaved from its synthesis resin whereas the other fragment of an antibody can be synthesized and subsequently cleaved from the resin, thereby exposing a terminal group which is functionally blocked on the other fragment. By peptide condensation reactions, these two fragments can be covalently joined via a peptide bond at their carboxyl and amino termini, respectively, to form an antibody, or fragment thereof (Grant G A (1992) Synthetic Peptides: A User Guide. W. H. Freeman and Co., N.Y. (1992); Bodansky M and Trost B., Ed. (1993) Principles of Peptide Synthesis. Springer-Verlag Inc., NY. Alternatively, the peptide or polypeptide is independently synthesized in vivo as described above. Once isolated, these independent peptides or polypeptides may be linked to form an antibody or fragment thereof via similar peptide condensation reactions.

For example, enzymatic ligation of cloned or synthetic peptide segments allow relatively short peptide fragments to be joined to produce larger peptide fragments, polypeptides or whole protein domains (Abrahmsen L et al., Biochemistry, 30:4151 (1991)). Alternatively, native chemical ligation of synthetic peptides can be utilized to synthetically construct large peptides or polypeptides from shorter peptide fragments. This method consists of a two step chemical reaction (Dawson et al. Synthesis of Proteins by Native Chemical Ligation. Science, 266:776-779 (1994)). The first step is the chemoselective reaction of an unprotected synthetic peptide-alpha-thioester with another unprotected peptide segment containing an amino-terminal Cys residue to give a thioester-linked intermediate as the initial covalent product. Without a change in the reaction conditions, this intermediate undergoes spontaneous, rapid intramolecular reaction to form a native peptide bond at the ligation site. Application of this native chemical ligation method to the total synthesis of a protein molecule is illustrated by the preparation of human interleukin 8 (IL-8) (Baggiolini M et al. (1992) FEBS Lett. 307:97-101; Clark-Lewis I et al., J. Biol. Chem., 269:16075 (1994); Clark-Lewis I et al., Biochemistry, 30:3128 (1991); Rajarathnam K et al., Biochemistry 33:6623-30 (1994)).

Alternatively, unprotected peptide segments are chemically linked where the bond formed between the peptide segments as a result of the chemical ligation is an unnatural (non-peptide) bond (Schnolzer, M et al. Science, 256:221 (1992)). This technique has been used to synthesize analogs of protein domains as well as large amounts of relatively pure proteins with full biological activity (deLisle Milton RC et al., Techniques in Protein Chemistry IV. Academic Press, New York, pp. 257-267 (1992)).

Also disclosed are fragments of antibodies which have bioactivity. The polypeptide fragments can be recombinant proteins obtained by cloning nucleic acids encoding the polypeptide in an expression system capable of producing the polypeptide fragments thereof, such as an adenovirus or baculovirus expression system. For example, one can determine the active domain of an antibody from a specific hybridoma that can cause a biological effect associated with the interaction of the antibody with Bob. For example, amino acids found to not contribute to either the activity or the binding specificity or affinity of the antibody can be deleted without a loss in the respective activity. For example, in various embodiments, amino or carboxy-terminal amino acids are sequentially removed from either the native or the modified non-immunoglobulin molecule or the immunoglobulin molecule and the respective activity assayed in one of many available assays. In another example, a fragment of an antibody comprises a modified antibody wherein at least one amino acid has been substituted for the naturally occurring amino acid at a specific position, and a portion of either amino terminal or carboxy terminal amino acids, or even an internal region of the antibody, has been replaced with a polypeptide fragment or other moiety, such as biotin, which can facilitate in the purification of the modified antibody. For example, a modified antibody can be fused to a maltose binding protein, through either peptide chemistry or cloning the respective nucleic acids encoding the two polypeptide fragments into an expression vector such that the expression of the coding region results in a hybrid polypeptide. The hybrid polypeptide can be affinity purified by passing it over an amylose affinity column, and the modified antibody receptor can then be separated from the maltose binding region by cleaving the hybrid polypeptide with the specific protease factor Xa. (See, for example, New England Biolabs Product Catalog, 1996, pg. 164.). Similar purification procedures are available for isolating hybrid proteins from eukaryotic cells as well.

The fragments, whether attached to other sequences or not, include insertions, deletions, substitutions, or other selected modifications of particular regions or specific amino acids residues, provided the activity of the fragment is not significantly altered or impaired compared to the nonmodified antibody or antibody fragment. These modifications can provide for some additional property, such as to remove or add amino acids capable of disulfide bonding, to increase its bio-longevity, to alter its secretory characteristics, etc. In any case, the fragment must possess a bioactive property, such as binding activity, regulation of binding at the binding domain, etc. Functional or active regions of the antibody may be identified by mutagenesis of a specific region of the protein, followed by expression and testing of the expressed polypeptide. Such methods are readily apparent to a skilled practitioner in the art and can include site-specific mutagenesis of the nucleic acid encoding the antigen. (Zoller M J et al. Nucl. Acids Res. 10:6487-500 (1982).

A variety of immunoassay formats may be used to select antibodies that selectively bind with a particular protein, variant, or fragment. For example, solid-phase ELISA immunoassays are routinely used to select antibodies selectively immunoreactive with a protein, protein variant, or fragment thereof. See Harlow and Lane. Antibodies, A Laboratory Manual. Cold Spring Harbor Publications, New York, (1988), for a description of immunoassay formats and conditions that could be used to determine selective binding. The binding affinity of a monoclonal antibody can, for example, be determined by the Scatchard analysis of Munson et al., Anal. Biochem., 107:220 (1980).

Also provided is an antibody reagent kit comprising containers of the monoclonal antibody or fragments thereof and one or more reagents for detecting binding of the antibody or fragment thereof to the Bob receptor molecule. The reagents can include, for example, fluorescent tags, enzymatic tags, or other tags. The reagents can also include secondary or tertiary antibodies or reagents for enzymatic reactions, wherein the enzymatic reactions produce a product that can be visualized.

(a) Antibody Conjugates

The disclosed antibodies, for example those to Bob or fragments thereof, as well as other molecules that bind Bob or fragments of Bob can be used, for example to reduce HIV enteropathy.

As discussed herein, it is understood that the disclosed antibodies can be conjugated to a variety of molecules. For example, the antibody can be coupled to a label which is detectable but which does not interfere with binding to the integrin receptors or fragments thereof. Although described primarily with reference to radioisotopes, especially indium (“In”), which is useful for diagnostic purposes, and yttrium (“Y”), which is cytotoxic, other substances which harm or inactivate cells can be substituted for the radioisotope. The antibodies may be unlabeled or labeled with a therapeutic agent. These agents can be coupled either directly or indirectly to the disclosed antibodies or substrate analogs. One example of indirect coupling is by use of a spacer moiety. These spacer moieties, in turn, can be either insoluble or soluble (Diener, et al., Science, 231:148, 1986) and can be selected to enable drug release from the antibodies or substrate analogs at the target site. Examples of therapeutic agents which can be coupled to the disclosed antibodies or analogs are drugs, radioisotopes, lectins, and toxins or agents which will covalently attach the antibody or substrate analog to the target or surrounding moelcules.

Crosslinking agents have two reactive functional groups and are classified as being homo or heterobifunctional. Examples of homobifunctional crosslinking agents include bismaleimidohexane (BMH) which is reactive with sulfhydryl groups (Chen, et al. J Biol Chem 266: 18237-18243 (1991) and ethylene glycolbis[succinimidylsucciate] EGS which is reactive with amino groups (Browning, et al., J. Immunol. 143: 1859-1867 (1989)). An example of a heterobifunctional crosslinker is m-maleimidobenzoyl-N-hydroxysuccinimide ester (MBS) (Myers, et al. J. Immunol. Meth. 121: 129-142 (1989)). These methodologies are simple and are commonly employed.

(2) Functional Nucleic Acids

Functional nucleic acids are nucleic acid molecules that have a specific function, such as binding a target molecule or catalyzing a specific reaction. Functional nucleic acid molecules can be divided into the following categories, which are not meant to be limiting. For example, functional nucleic acids include antisense molecules, aptamers, ribozymes, triplex forming molecules, and external guide sequences. The functional nucleic acid molecules can act as affectors, inhibitors, modulators, and stimulators of a specific activity possessed by a target molecule, or the functional nucleic acid molecules can possess a de novo activity independent of any other molecules.

Functional nucleic acid molecules can interact with any macromolecule, such as DNA, RNA, polypeptides, or carbohydrate chains. Thus, functional nucleic acids can interact with the mRNA of Bob or the genomic DNA of Bob or they can interact with the polypeptide Bob, for example, with fragments of the Bob polypeptide comprising either SEQ D NO:1, SEQ ID NO:2, SEQ ID NO:3, SEQ ID NO:4, SEQ ID NO:5, or SEQ ID NO:6. Often functional nucleic acids are designed to interact with other nucleic acids based on sequence homology between the target molecule and the functional nucleic acid molecule. In other situations, the specific recognition between the functional nucleic acid molecule and the target molecule is not based on sequence homology between the functional nucleic acid molecule and the target molecule, but rather is based on the formation of tertiary structure that allows specific recognition to take place.

Antisense molecules are designed to interact with a target nucleic acid molecule through either canonical or non-canonical base pairing. The interaction of the antisense molecule and the target molecule is designed to promote the destruction of the target molecule through, for example, RNAseH mediated RNA-DNA hybrid degradation. Alternatively the antisense molecule is designed to interrupt a processing function that normally would take place on the target molecule, such as transcription or replication. Antisense molecules can be designed based on the sequence of the target molecule. Numerous methods for optimization of antisense efficiency by finding the most accessible regions of the target molecule exist. Exemplary methods would be in vitro selection experiments and DNA modification studies using DMS and DEPC. It is preferred that antisense molecules bind the target molecule with a dissociation constant (k_{d}) less than 10⁻⁶. It is more preferred that antisense molecules bind with a k_{d} less than 10⁻⁸. It is also more preferred that the antisense molecules bind the target moelcule with a k_{d} less than 10⁻¹⁰. It is also preferred that the antisense molecules bind the target molecule with a k_{d} less than 10⁻¹². A representative sample of methods and techniques which aid in the design and use of antisense molecules can be found in the following non-limiting list of U.S. Pat. Nos. 5,135,917, 5,294,533, 5,627,158, 5,641,754, 5,691,317, 5,780,607, 5,786,138, 5,849,903, 5,856,103, 5,919,772, 5,955,590, 5,990,088, 5,994,320, 5,998,602, 6,005,095, 6,007,995, 6,013,522, 6,017,898, 6,018,042, 6,025,198, 6,033,910, 6,040,296, 6,046,004, 6,046,319, and 6,057,437.

Aptamers are molecules that interact with a target molecule, preferably in a specific way. Typically aptamers are small nucleic acids ranging from 15-50 bases in length that fold into defined secondary and tertiary structures, such as stem-loops or G-quartets. Aptamers can bind small molecules, such as ATP (U.S. Pat. No. 5,631,146) and theophiline (U.S. Pat. No. 5,580,737), as well as large molecules, such as reverse transcriptase (U.S. Pat. No. 5,786,462) and thrombin (U.S. Pat. No. 5,543,293). Aptamers can bind very tightly with kds from the target molecule of less than 10−12 M. It is preferred that the aptamers bind the target molecule with a kd less than 10−6. It is more preferred that the aptamers bind the target molecule with a kd less than 10−8. It is also more preferred that the aptarners bind the target molecule with a kd less than 10−10. It is also preferred that the aptamers bind the target molecule with a kd less than 10−12. Aptamers can bind the target molecule with a very high degree of specificity. For example, aptamers have been isolated that have greater than a 10000 fold difference in binding affinities between the target molecule and another molecule that differ at only a single position on the molecule (U.S. Pat. No. 5,543,293). It is preferred that the aptarner have a kd with the target molecule at least 10 fold lower than the kd with a background binding molecule. It is more preferred that the aptamer have a kd with the target molecule at least 100 fold lower than the kd with a background binding molecule. It is more preferred that the aptamer have a kd with the target molecule at least 1000 fold lower than the kd with a background binding molecule. It is preferred that the aptamer have a kd with the target molecule at least 10000 fold lower than the kd with a background binding molecule. It is preferred when doing the comparison for a polypeptide for example, that the background molecule be a different polypeptide. For example, when determining the specificity of Bob or Bob fragment aptamers, the background protein could be bovine serum albumin. Representative examples of how to make and use aptamers to bind a variety of different target molecules can be found in the following non-limiting list of U.S. Pat. Nos. 5,476,766, 5,503,978, 5,631,146, 5,731,424, 5,780,228, 5,792,613, 5,795,721, 5,846,713, 5,858,660, 5,861,254, 5,864,026, 5,869,641, 5,958,691, 6,001,988, 6,011,020, 6,013,443, 6,020,130, 6,028,186, 6,030,776, and 6,051,698.

Ribozymes are nucleic acid molecules that are capable of catalyzing a chemical reaction, either intramolecularly or intermolecularly. Ribozymes are thus catalytic nucleic acid. It is preferred that the ribozymes catalyze intermolecular reactions. There are a number of different types of ribozymes that catalyze nuclease or nucleic acid polymerase type reactions which are based on ribozymes found in natural systems, such as hammerhead ribozymes, (for example, but not limited to the following U.S. Pat. Nos. 5,334,711, 5,436,330, 5,616,466, 5,633,133, 5,646,020, 5,652,094, 5,712,384, 5,770,715, 5,856,463, 5,861,288, 5,891,683, 5,891,684, 5,985,621, 5,989,908, 5,998,193, 5,998,203, WO 9858058 by Ludwig and Sproat, WO 9858057 by Ludwig and Sproat, and WO 9718312 by Ludwig and Sproat) hairpin ribozymes (for example, but not limited to the following U.S. Pat. Nos. 5,631,115, 5,646,031, 5,683,902, 5,712,384, 5,856,188, 5,866,701, 5,869,339, and 6,022,962), and tetrahymena ribozymes (for example, but not limited to the following U.S. Pat. Nos, 5,595,873 and 5,652,107). There are also a number of ribozymes that are not found in natural systems, but which have been engineered to catalyze specific reactions de novo (for example, but not limited to the following U.S. Pat. Nos. 5,580,967, 5,688,670, 5,807,718, and 5,910,408). Preferred ribozymes cleave RNA or DNA substrates, and more preferably cleave RNA substrates. Ribozymes typically cleave nucleic acid substrates through recognition and binding of the target substrate with subsequent cleavage. This recognition is often based mostly on canonical or non-canonical base pair interactions. This property makes ribozymes particularly good candidates for target specific cleavage of nucleic acids because recognition of the target substrate is based on the target substrates sequence. Representative examples of how to make and use ribozymes to catalyze a variety of different reactions can be found in the following non-limiting list of U.S. Pat. Nos. 5,646,042, 5,693,535, 5,731,295, 5,811,300, 5,837,855, 5,869,253, 5,877,021, 5,877,022, 5,972,699, 5,972,704, 5,989,906, and 6,017,756.

Triplex forming functional nucleic acid molecules are molecules that can interact with either double-stranded or single-stranded nucleic acid. When triplex molecules interact with a target region, a structure called a triplex is formed, in which there are three strands of DNA forming a complex dependant on both Watson-Crick and Hoogsteen base-pairing. Triplex molecules are preferred because they can bind target regions with high affinity and specificity. It is preferred that the triplex forming molecules bind the target molecule with a k_{d} less than 10⁻⁶. It is more preferred that the triplex forming molecules bind with a k_{d} less than 10⁻⁸. It is also more preferred that the triplex forming molecules bind the target moelcule with a k_{d} less than 10⁻¹⁰. It is also preferred that the triplex forming molecules bind the target molecule with a k_{d} less than 10⁻¹². Representative examples of how to make and use triplex forming molecules to bind a variety of different target molecules can be found in the following non-limiting list of U.S. Pat. Nos. 5,176,996, 5,645,985, 5,650,316, 5,683,874, 5,693,773, 5,834,185, 5,869,246, 5,874,566, and 5,962,426.

External guide sequences (EGSs) are molecules that bind a target nucleic acid molecule forming a complex, and this complex is recognized by RNase P, which cleaves the target molecule. EGSs can be designed to specifically target a RNA molecule of choice. RNAse P aids in processing transfer RNA (tRNA) within a cell. Bacterial RNAse P can be recruited to cleave virtually any RNA sequence by using an EGS that causes the target RNA:EGS complex to mimic the natural tRNA substrate. (WO 92/03566 by Yale, and Forster and Altman, Science 238:407-409 (1990)).

Similarly, eukaryotic EGS/RNAse P-directed cleavage of RNA can be utilized to cleave desired targets within eukarotic cells. (Yuan et al., Proc. Natl. Acad. Sci. USA 89:8006-8010 (1992); WO 93/22434 by Yale; WO 95/24489 by Yale; Yuan and Altman, EMBO J. 14:159-168 (1995), and Carrara et al., Proc. Natl. Acad. Sci. (USA) 92:2627-2631 (1995)). Representative examples of how to make and use EGS molecules to facilitate cleavage of a variety of different target molecules be found in the following non-limiting list of U.S. Pat. Nos. 5,168,053, 5,624,824, 5,683,873, 5,728,521, 5,869,248, and 5,877,162

(3) Compositions Identified by Screening with Disclosed Compositions/Combinatorial Chemistry

(a) Combinatorial Chemistry

The disclosed compositions can be used as targets for any combinatorial technique to identify molecules or macromolecular molecules that interact with the disclosed compositions in a desired way. The nucleic acids, peptides, and related molecules disclosed herein can be used as targets for the combinatorial approaches. Also disclosed are the compositions that are identified through combinatorial techniques or screening techniques in which the compositions disclosed in SEQ ID NOS:1-10 or portions thereof, are used as the target in a combinatorial or screening protocol.

It is understood that when using the disclosed compositions in combinatorial techniques or screening methods, molecules, such as macromolecular molecules, will be identified that have particular desired properties such as inhibition or stimulation or the target molecule's function. The molecules identified and isolated when using the disclosed compositions, such as, SEQ ID NOs:1-6, are also disclosed. Thus, the products produced using the combinatorial or screening approaches that involve the disclosed compositions, such as, SEQ ID NOs:1-6, are also considered herein disclosed.

Combinatorial chemistry includes but is not limited to all methods for isolating small molecules or macromolecules that are capable of binding either a small molecule or another macromolecule, typically in an iterative process. Proteins, oligonucleotides, and sugars are examples of macromolecules. For example, oligonucleotide molecules with a given function, catalytic or ligand-binding, can be isolated from a complex mixture of random oligonucleotides in what has been referred to as “in vitro genetics” (Szostak, TIBS 19:89, 1992). One synthesizes a large pool of molecules bearing random and defined sequences and subjects that complex mixture, for example, approximately 10¹⁵ individual sequences in 100 μg of a 100 nucleotide RNA, to some selection and enrichment process. Through repeated cycles of affinity chromatography and PCR amplification of the molecules bound to the ligand on the column, Ellington and Szostak (1990) estimated that 1 in 10¹⁰ RNA molecules folded in such a way as to bind a small molecule dyes. DNA molecules with such ligand-binding behavior have been isolated as well (Ellington and Szostak, 1992; Bock et al, 1992). Techniques aimed at similar goals exist for small organic molecules, proteins, antibodies and other macromolecules known to those of skill in the art. Screening sets of molecules for a desired activity whether based on small organic libraries, oligonucleotides, or antibodies is broadly referred to as combinatorial chemistry. Combinatorial techniques are particularly suited for defining binding interactions between molecules and for isolating molecules that have a specific binding activity, often called aptamers when the macromolecules are nucleic acids.

There are a number of methods for isolating proteins which either have de novo activity or a modified activity. For example, phage display libraries have been used to isolate numerous peptides that interact with a specific target. (See for example, U.S. Pat. Nos. 6,031,071; 5,824,520; 5,596,079; and 5,565,332 which are herein incorporated by reference at least for their material related to phage display and methods relate to combinatorial chemistry)

A preferred method for isolating proteins that have a given function is described by Roberts and Szostak (Roberts R. W. and Szostak J. W. Proc. Natl. Acad. Sci. USA, 94(23)12997-302 (1997). This combinatorial chemistry method couples the functional power of proteins and the genetic power of nucleic acids. An RNA molecule is generated in which a puromycin molecule is covalently attached to the 3′-end of the RNA molecule. An in vitro translation of this modified RNA molecule causes the correct protein, encoded by the RNA to be translated. In addition, because of the attachment of the puromycin, a peptdyl acceptor which cannot be extended, the growing peptide chain is attached to the puromycin which is attached to the RNA. Thus, the protein molecule is attached to the genetic material that encodes it. Normal in vitro selection procedures can now be done to isolate functional peptides. Once the selection procedure for peptide function is complete traditional nucleic acid manipulation procedures are performed to amplify the nucleic acid that codes for the selected functional peptides. After amplification of the genetic material, new RNA is transcribed with puromycin at the 3′-end, new peptide is translated and another functional round of selection is performed. Thus, protein selection can be performed in an iterative manner just like nucleic acid selection techniques. The peptide which is translated is controlled by the sequence of the RNA attached to the puromycin. This sequence can be anything from a random sequence engineered for optimum translation (i.e. no stop codons etc.) or it can be a degenerate sequence of a known RNA molecule to look for improved or altered function of a known peptide. The conditions for nucleic acid amplification and in vitro translation are well known to those of ordinary skill in the art and are preferably performed as in Roberts and Szostak (Roberts R. W. and Szostak J. W. Proc. Natl. Acad. Sci. USA, 94(23)12997-302 (1997)).

Another preferred method for combinatorial methods designed to isolate peptides is described in Cohen et al. (Cohen B. A., et al., Proc. Natl. Acad. Sci. USA 95(24):14272-7 (1998)). This method utilizes and modifies two-hybrid technology. Yeast two-hybrid systems are useful for the detection and analysis of protein:protein interactions. The two-hybrid system, initially described in the yeast Saccharomyces cerevisiae, is a powerful molecular genetic technique for identifying new regulatory molecules, specific to the protein of interest (Fields and Song, Nature 340:245-6 (1989)). Cohen et al., modified this technology so that novel interactions between synthetic or engineered peptide sequences could be identified which bind a molecule of choice. The benefit of this type of technology is that the selection is done in an intracellular environment. The method utilizes a library of peptide molecules that attached to an acidic activation domain. A peptide of choice, for example, gp120 interacting fragments of Bob set forth in SEQ ID NOs:1-6 is attached to a DNA binding domain of a transcriptional activation protein, such as Gal 4. By performing the Two-hybrid technique on this type of system, molecules that bind the gp120 interacting fragment of Bob set forth in SEQ ID NOs:1-6 can be identified.

Using methodology well known to those of skill in the art, in combination with various combinatorial libraries, one can isolate and characterize those small molecules or macromolecules, which bind to or interact with the desired target. The relative binding affinity of these compounds can be compared and optimum compounds identified using competitive binding studies, which are well known to those of skill in the art.

Techniques for making combinatorial libraries and screening combinatorial libraries to isolate molecules which bind a desired target are well known to those of skill in the art. Representative techniques and methods can be found in but are not limited to U.S. Pat. Nos. 5,084,824, 5,288,514, 5,449,754, 5,506,337, 5,539,083, 5,545,568, 5,556,762, 5,565,324, 5,565,332, 5,573,905, 5,618,825, 5,619,680, 5,627,210, 5,646,285, 5,663,046, 5,670,326, 5,677,195, 5,683,899, 5,688,696, 5,688,997, 5,698,685, 5,712,146, 5,721,099, 5,723,598, 5,741,713, 5,792,431, 5,807,683, 5,807,754, 5,821,130, 5,831,014, 5,834,195, 5,834,318, 5,834,588, 5,840,500, 5,847,150, 5,856,107, 5,856,496, 5,859,190, 5,864,010, 5,874,443, 5,877,214, 5,880,972, 5,886,126, 5,886,127, 5,891,737, 5,916,899, 5,919,955, 5,925,527, 5,939,268, 5,942,387, 5,945,070, 5,948,696, 5,958,702, 5,958,792, 5,962,337, 5,965,719, 5,972,719, 5,976,894, 5,980,704, 5,985,356, 5,999,086, 6,001,579, 6,004,617, 6,008,321, 6,017,768, 6,025,371, 6,030,917, 6,040,193, 6,045,671, 6,045,755, 6,060,596, and 6,061,636.

Combinatorial libraries can be made from a wide array of molecules using a number of different synthetic techniques. For example, libraries containing fused 2,4-pyrimidinediones (U.S. Pat. No. 6,025,371) dihydrobenzopyrans (U.S. Pat. No. 6,017,768 and 5,821,130), amide alcohols (U.S. Pat. No. 5,976,894), hydroxy-amino acid amides (U.S. Pat. No. 5,972,719) carbohydrates (U.S. Pat. No. 5,965,719), 1,4-benzodiazepin-2,5-diones (U.S. Pat. No. 5,962,337), cyclics (U.S. Pat. No. 5,958,792), biaryl amino acid amides (U.S. Pat. No. 5,948,696), thiophenes (U.S. Pat. No. 5,942,387), tricyclic Tetrahydroquinolines (U.S. Pat. No. 5,925,527), benzofurans (U.S. Pat. No. 5,919,955), isoquinolines (U.S. Pat. No. 5,916,899), hydantoin and thiohydantoin (U.S. Pat. No. 5,859,190), indoles (U.S. Pat. No. 5,856,496), imidazol-pyrido-indole and imidazol-pyrido-benzothiophenes (U.S. Pat. No. 5,856,107) substituted 2-methylene-2,3-dihydrothiazoles (U.S. Pat. No. 5,847,150), quinolines (U.S. Pat. No. 5,840,500), PNA (U.S. Pat. No. 5,831,014), containing tags (U.S. Pat. No. 5,721,099), polyketides (U.S. Pat. No. 5,712,146), morpholino-subunits (U.S. Pat. No. 5,698,685 and 5,506,337), sulfamides (U.S. Pat. No. 5,618,825), and benzodiazepines (U.S. Pat. No. 5,288,514).

Screening molecules similar to those disclosed herein for inhibition of gp120-Bob interaction, particularly screening for molecules that interact with fragments of Bob, set forth in SEQ ID NOs:1-6 is a method of isolating desired compounds.

As used herein combinatorial methods and libraries included traditional screening methods and libraries as well as methods and libraries used in interative processes.

Also disclosed are systems for isolating and producing molecules that bind Bob and inhibit gp120-Bob binding, as well as isolating and producing molecules that bind Bob and inhibit the effects of gp120 induced activation of cells.

As disclosed herein the interactions between gp120 and Bob cause a variety of activation events to occur. These events include calcium signaling induced by gp120-Bob binding. Thus, desired molecules are those that bind Bob and inhibit CA Flux in cells caused by gp120. Typically, to assay for calcium fluxes appropriate amounts of pure protein is required. Use of whole virus has been thwarted because HIV is traditionally grown in the presence of fetal bovine serum and phytohemaglutinin, both of which induce calcium fluxes which could interfere with a direct assay of culture supernates.

Disclosed herein are methods that allow for the direct assay of HIV culture supernatant to determine if calcium activation has occurred or been reduced. The method comprises the steps of growing the HIV in any medium, called here “amplification medium” including mediums which themselves can induce calcium flux. Typical mediums of this nature, would be for example, RPMI 1640 with 10% fetal calf serum, 5 micrograms/ml phytohemaglutinin-P, and 20 u/ml IL-2. The HIV can be grown in this medium approximately 3 days, since a cycle of productive infection is generally 4 to 5 days under optimal conditions in previously activated cells. Under optimal conditions (the cells are in an activated state from activation inducing agents) the time of growth in the amplification medium is limited by the understanding that productive infection of cells by HIV lasts typically less than about 6 days, and usually about 4-5 days, and the cells also need to have time to grow and become productively infected, which is usually at least about 1, 2, 3, 4, or 5 days. Typically the amplification medium is switched to the assay medium prior to significant viral budding or viral protein shedding. This can be determined by assaying for p24. Typically transfer will occur prior to the presence of greater than about 10 ng/ml p24. In other embodiments, the transfer from the amplification medium to the assay medium takes place prior to completion of viral and viral protein shedding. Thus, growth in the amplifcation medium typically occurs for at least about 1, 2, 3, 4, or 5 days. Following this growth in regular medium, the medium is switched to a medium that can allow for direct assaying of calcium flux, the “assay medium.” An example of an assay medium is AIM-V. This can be used in conjunction with 20 u/ml IL-2 but no serum or phytohemaglutinin. Any medium functioning as AIM-V can be used. This medium does not induce calcium fluxes, allowing the direct testing of culture supernates for Bob activation in an appropriately transfected cell line.

Disclosed are methods comprising first culturing HIV in an amplification medium, and then culturing the HIV in an assay medium.

In the calcium flux assay any monitoring means can be used, such as radioactivity or fluorescence. For example, Fluo-4, Fluo-3, and Indo-1 and others can be used. These are in the cytoplasm and they become more fluorescent (or there is an alteration such as a shift in the wavelength of the fluorescence) when they bind calcium that enters the cytoplasm. There are high throughput instruments that can measure 96 or 384 wells for calcium flux at once. One such device is called a “FLIPR” and is made by Molecular Devices, Sunnyvale Calif. (www.moldev.com).

In certain embodiments the amplification medium comprises RPMI 1640 with 10% fetal calf serum and phytohemaglutinin. Any other medium which contains serum and either phytohemaglutinin or some other substance which induces a calcium flux can also be used. For short term culture, the IL-2 is not needed. Typically IL-2 is used for the proliferation and the very survival of T cells in culture for more than a about 3 days.

The methods of direct testing of calcium flux can be utilized with combinatorial approaches to identify molecules that bind Bob and reduce Bob-gp120 interactions. These direct testing methods can also be used to assay and compare compositions that bind Bob, for their ability to inhibit calcium flux induced by gp120-Bob interactions.

Disclosed are methods of culturing HIV comprising first culturing HIV in an amplification medium, and then culturing the HIV in an assay medium wherein the assay medium does not elicit a calcium flux.

Also disclosed are methods comprising and further comprising infecting cells with the HIV grown in the assay medium producing infected cells.

Disclosed are methods, wherein the cells comprise Ghost (3) cells, human osteosarcoma cells, chinese hamster ovary (CHO) cells, HEK293 cells, Jurkat cells, HT-29 cells, HCT116 cells, DLD1 cells, intestinal cells, human lymphocytes, macrophages, peripheral blood mononuclear cells, renal tubular cell lines, or Daudi cells, and wherein the cells express Bob.

Disclosed are methods, wherein the cells comprise Ghost (3) cells, human osteosarcoma cells, chinese hamster ovary (CHO) cells, HEK293 cells, Jurkat cells, HT-29 cells, HCT116 cells, DLD1 cells, intestinal cells, human lymphocytes, macrophages, peripheral blood mononuclear cells, renal tubular cell lines, or Daudi cells, and wherein the cells comprise Bob.

Disclosed are methods comprising and, further comprising assaying the infected cells for calcium flux.

Disclosed are methods, wherein assaying the infected cells for calcium flux comprises assaying increased cytosolic calcium content resulting from gp120-induced, Bob-mediated activation.

Disclosed are methods, comprising and further comprising incubating the infected cells with a potential inhibitor of Bob-gp120 binding.

Disclosed are methods, wherein the amplification medium induces calcium flux.

Disclosed are methods, wherein the amplification medium comprises serum.

Disclosed are methods, wherein the serum is bovine serum.

Disclosed are methods, wherein the amplification medium comprises phytohemaglutinin.

Disclosed are methods, wherein the amplification medium further comprises bovine serum.

Disclosed are methods, wherein the HIV is cultured in the amplification medium for at least 3 days.

Disclosed are methods, wherein the amplification medium comprises RPMI 1640 with 10% fetal calf serum and phytohemaglutinin.

Disclosed are methods, wherein the assay medium does not comprise bovine serum or phytohemaglutinin.

Disclosed are methods, wherein the assay medium comprises AIM-V.

Disclosed are methods, wherein the assay medium comprises 20 u/ml IL-2.

(b) Computer Assisted Drug Design

The disclosed compositions can be used as targets for any molecular modeling technique to identify either the structure of the disclosed compositions or to identify potential or actual molecules, such as small molecules, which interact in a desired way with the disclosed compositions. The nucleic acids, peptides, and related molecules disclosed herein, such as the polypeptide Bob, for example, or fragments of the Bob polypeptide comprising either SEQ ID NO:1, SEQ ID NO:2, SEQ ID NO:3, SEQ ID NO:4, SEQ ID NO:5, or SEQ ID NO:6, can be used as targets in any molecular modeling program or approach.

It is understood that when using the disclosed compositions in modeling techniques, molecules, such as macromolecular molecules, will be identified that have particular desired properties such as inhibition or stimulation or the target molecule's function. The molecules identified and isolated when using the disclosed compositions, such as, the disclosed antibodies, are also disclosed. Thus, the products produced using the molecular modeling approaches that involve the disclosed compositions, such as, the polypeptide Bob or fragments of the Bob polypeptide comprising either SEQ ID NO:1, SEQ ID NO:2, SEQ ID NO:3, SEQ ID NO:4, SEQ ID NO:5, or SEQ ID NO:6 or molecules that interact with the polypeptide Bob or fragments of the Bob polypeptide comprising either SEQ ID NO:1, SEQ ID NO:2, SEQ ID NO:3, SEQ ID NO:4, SEQ ID NO:5, or SEQ ID NO:6, are also considered herein disclosed.

Thus, one way to isolate molecules that bind a molecule of choice is through rational design. This is achieved through structural information and computer modeling. Computer modeling technology allows visualization of the three-dimensional atomic structure of a selected molecule and the rational design of new compounds that will interact with the molecule. The three-dimensional construct typically depends on data from x-ray crystallographic analyses or NMR imaging of the selected molecule. The molecular dynamics require force field data. The computer graphics systems enable prediction of how a new compound will link to the target molecule and allow experimental manipulation of the structures of the compound and target molecule to perfect binding specificity. Prediction of what the molecule-compound interaction will be when small changes are made in one or both requires molecular mechanics software and computationally intensive computers, usually coupled with user-friendly, menu-driven interfaces between the molecular design program and the user.

Examples of molecular modeling systems are the CHARMm and QUANTA programs, Polygen Corporation, Waltham, Mass. CHARMm performs the energy minimization and molecular dynamics functions. QUANTA performs the construction, graphic modeling and analysis of molecular structure. QUANTA allows interactive construction, modification, visualization, and analysis of the behavior of molecules with each other.

A number of articles review computer modeling of drugs interactive with specific proteins, such as Rotivinen, et al., 1988 Acta Pharmaceutica Fennica 97, 159-166; Ripka, New Scientist 54-57 (Jun. 16, 1988); McKinaly and Rossmann, 1989 Annu. Rev. Pharmacol. Toxiciol. 29, 111-122; Perry and Davies, OSAR: Quantitative Structure-Activity Relationships in Drug Design pp. 189-193 (Alan R. Liss, Inc. 1989); Lewis and Dean, 1989 Proc. R. Soc. Lond. 236, 125-140 and 141-162; and, with respect to a model enzyme for nucleic acid components, Askew, et al., 1989 J. Am. Chem. Soc. 111, 1082-1090. Other computer programs that screen and graphically depict chemicals are available from companies such as BioDesign, Inc., Pasadena, Calif., Allelix, Inc, Mississauga, Ontario, Canada, and Hypercube, Inc., Cambridge, Ontario. Although these are primarily designed for application to drugs specific to particular proteins, they can be adapted to design of molecules specifically interacting with specific regions of DNA or RNA, once that region is identified.

Although described above with reference to design and generation of compounds which could alter binding, one could also screen libraries of known compounds, including natural products or synthetic chemicals, and biologically active materials, including proteins, for compounds which alter substrate binding or enzymatic activity.

c) Gp120

Unless specifically indicated, gp120 refers to the envelope surface protein of HIV. Gp 120 can come from any strain of HIV. Activation by gp120 includes G protein activation and its consequences, which are known to include inositol triphosphate/diacylglycerol signaling, calcium flux, protein kinase C activation, loss of microtubules, cyclic AMP production, or other signals that can be directly or indirectly related to G protein activation. Specifically, strains IIIB, LAI, CM235, and SEN are known to cause activation in the disclosed assays and the Bob 37 antibody has been shown to inhibit. Four of eight HIV strains tested activated Bob. Cyclic AMP is another molecule which can be activated by HIV through G-protein coupled cascades.

(1) Target gp120 Region

Synthetic soluble analogs of galactosylceramide (GalCer) bind to the V3 domain of HIV-1 gp120 and inhibit HIV-1-induced fusion and entry. Fantini et al. J Biol Chem 1997;272(11):7245-52)). CA-52 has been shown to bind a specific conserved sequence in gp120 that has the sequence “GPGRAF” (SEQ ID NO:) 15 which is amino acids 317 to 322 (in the V3 loop region) of the consensus sequence for the gp120 protein. CA-52 inhibits binding by two monoclonal antibodies, and recognizes the overlapping sequences “IQRGPG” (SEQ ID NO:16) and “GPGRAFVTI” (SEQ ID NO:17). Heparin is also known to bind the amino half of the V3 loop. The V3 loop is a region of gp120 that is among the least glycosylated, most antigenic, and among the most varied regions among different strains. The V3 loop is where gp120 binds CD4, the coreceptor used for viral fusion glycosphingolipids such as galactosyl ceramide. Fantini et al. J Biol Chem 1997;272(11):7245-52)).

CA-52 is an analog of galactosyl ceramide, and it strongly inhibits the calcium activation, suggesting that this small region is or is adjacent to the binding site used for Bob because Bob has, as is disclosed herein, antigenic similarity to galactosyl ceramide. This indicates that “GPGRAF” and/or a nearby sequence in the V3 loop of gp120 can bind either galactosyl ceramide or Bob. Galactosyl ceramide and Bob both reside in glycosphyngolipid-enriched microdomains (also called rafts), and the rafts are where viral fusion occurs. The G proteins, which are activated by Bob and the other G protein coupled receptors activate, are also known to be in rafts.

Double immunostaining of colonic tissue sections, containing lymphocytes and colonic epithelial cells, was performed with Cholera toxin FITC (cholera toxin binds GM1, another glycosphingolipid that is used as a marker for rafts) and antiBob37. Confocal microscopy showed that both had essentially identical granular to clumped membrane staining, although Bob immunostaining was also granular and cytoplasmic in some colonic epithelial cells. Thus Bob is found in rafts, and shuttling between cytoplasmic and raft membrane binding is common in G protein coupled receptors and this is a way their activity can be regulated.

There is a distinction between activation and infection: 1) antiBob but not antiCXCR4 blocks calcium signaling, while antiCXCR4 but not antiBob substantially blocks infection with the IIIB HIV-1 strain, 2) both the X4 (CXCR4-using for viral fusion, like IIIB) and R5 (CCR5-using for viral fusion, like SEN and CM235 strains) can activate Bob, demonstrating that different receptors are used for activation than for viral fusion, and 3) there are strains that activate Bob without infecting HT-29 cells (the SEN viral strain) or that infect HT-29 cells without having the activation effect (the 89.6 viral strain).

Immunoprecipitates of gp120 that has been allowed to bind appropriate cells, produce coimmunoprecipitates specifically with one strain or another. For example, a 36 kD protein corresponding to Bob coimmunoprecipitates along with the IIIB strain of gp120.

There are a number of gp120 V3 sequences in the vicinity of CA-52/Bob binding region (amino acids 315 to 322 of gp120, SEQ ID NO:7 For example, the sequence QRGPGRAF from IIIB gp120 and the analogous sequence of PIGPGQAF from CM235 gp120 are. It is also known that peptides having the sequence RIGPGQVF (93TH975, gp120), YIGPGRAF (SF2, gp120), and HIGPGRAF (MN, gp120) do not activate Bob.

In certain embodiments, gp120 sequences that activate Bob have a small, uncharged amino acid at residue 315 (or the analogous residue), then an (R/I)GP(G/Q)RAF. Those gp120 sequences that typically do not activate Bob have a large polar or charged amino acid at position 315 (or the analogous residue). It is likely the valine₃₂₁ of strain 93TH975 also may hinder binding.

d) Composition Characteristics

(1) Peptides

(a) Protein Variants

As discussed herein there are numerous variants of the Bob protein and gp120 protein that are known and herein contemplated. In addition, to the known functional gp120 strain variants and Bob variant there are derivatives of the gp120 and Bob proteins which also function in the disclosed methods and compositions. Protein variants and derivatives are well understood to those of skill in the art and in can involve amino acid sequence modifications. For example, amino acid sequence modifications typically fall into one or more of three classes: substitutional, insertional or deletional variants. Insertions include amino and/or carboxyl terminal fusions as well as intrasequence insertions of single or multiple amino acid residues. Insertions ordinarily will be smaller insertions than those of amino or carboxyl terminal fusions, for example, on the order of one to four residues. Immunogenic fusion protein derivatives, such as those described in the examples, are made by fusing a polypeptide sufficiently large to confer immunogenicity to the target sequence by cross-linking in vitro or by recombinant cell culture transformed with DNA encoding the fusion. Deletions are characterized by the removal of one or more amino acid residues from the protein sequence. Typically, no more than about from 2 to 6 residues are deleted at any one site within the protein molecule. These variants ordinarily are prepared by site specific mutagenesis of nucleotides in the DNA encoding the protein, thereby producing DNA encoding the variant, and thereafter expressing the DNA in recombinant cell culture. Techniques for making substitution mutations at predetermined sites in DNA having a known sequence are well known, for example M13 primer mutagenesis and PCR mutagenesis. Amino acid substitutions are typically of single residues, but can occur at a number of different locations at once; insertions usually will be on the order of about from 1 to 10 amino acid residues; and deletions will range about from 1 to 30 residues. Deletions or insertions preferably are made in adjacent pairs, i.e. a deletion of 2 residues or insertion of 2 residues. Substitutions, deletions, insertions or any combination thereof may be combined to arrive at a final construct. The mutations must not place the sequence out of reading frame and preferably will not create complementary regions that could produce secondary mRNA structure. Substitutional variants are those in which at least one residue has been removed and a different residue inserted in its place. Such substitutions generally are made in accordance with the following Tables 1 and 2 and are referred to as conservative substitutions.

TABLE 1
Amino Acid Abbreviations

| | |
|---|---|
| Amino Acid | Abbreviations |

| | | |
|---|---|---|
| alanine | Ala | A |
| allosoleucine | AIle | |
| arginine | Arg | R |
| asparagine | Asn | N |
| aspartic acid | Asp | D |
| cysteine | Cys | C |
| glutamic acid | Glu | E |
| glutamine | Gln | K |
| glycine | Gly | G |
| histidine | His | H |
| isolelucine | Ile | I |
| leucine | Leu | L |
| lysine | Lys | K |
| phenylalanine | Phe | F |
| proline | Pro | P |
| pyroglutamic acid | pGlu | |
| serine | Ser | S |
| threonine | Thr | T |
| tyrosine | Tyr | Y |
| tryptophan | Trp | W |
| valine | Val | V |

TABLE 2
Amino Acid Substitutions

| | Exemplary Conservative Substitutions, |
|---|---|
| Original Residue | others are known in the art. |
| Ala | ser |
| Arg | lys, gln |
| Asn | gln; his |
| Asp | glu |
| Cys | ser |
| Gln | asn, lys |
| Glu | asp |
| Gly | pro |
| His | asn; gln |
| Ile | leu; val |
| Leu | ile; val |
| Lys | arg; gln; |
| Met | Leu; ile |
| Phe | met; leu; tyr |
| Ser | thr |
| Thr | ser |
| Trp | tyr |
| Tyr | trp; phe |
| Val | ile; leu |

Substantial changes in function or immunological identity are made by selecting substitutions that are less conservative than those in Table 2, i.e., selecting residues that differ more significantly in their effect on maintaining (a) the structure of the polypeptide backbone in the area of the substitution, for example as a sheet or helical conformation, (b) the charge or hydrophobicity of the molecule at the target site or (c) the bulk of the side chain. The substitutions which in general are expected to produce the greatest changes in the protein properties will be those in which (a) a hydrophilic residue, e.g. seryl or threonyl, is substituted for (or by) a hydrophobic residue, e.g. leucyl, isoleucyl, phenylalanyl, valyl or alanyl; (b) a cysteine or proline is substituted for (or by) any other residue; (c) a residue having an electropositive side chain, e.g., lysyl, arginyl, or histidyl, is substituted for (or by) an electronegative residue, e.g., glutamyl or aspartyl; or (d) a residue having a bulky side chain, e.g., phenylalanine, is substituted for (or by) one not having a side chain, e.g., glycine, in this case, (e) by increasing the number of sites for sulfation and/or glycosylation.

For example, the replacement of one amino acid residue with another that is biologically and/or chemically similar is known to those skilled in the art as a conservative substitution. For example, a conservative substitution would be replacing one hydrophobic residue for another, or one polar residue for another. The substitutions include combinations such as, for example, Gly, Ala; Val, Ile, Leu; Asp, Glu; Asn, Gln; Ser, Thr; Lys, Arg; and Phe, Tyr. Such conservatively substituted variations of each explicitly disclosed sequence are included within the mosaic polypeptides provided herein.

Substitutional or deletional mutagenesis can be employed to insert sites for N-glycosylation (Asn-X-Thr/Ser) or O-glycosylation (Ser or Thr). Deletions of cysteine or other labile residues also may be desirable. Deletions or substitutions of potential proteolysis sites, e.g. Arg, is accomplished for example by deleting one of the basic residues or substituting one by glutaminyl or histidyl residues.

Certain post-translational derivatizations are the result of the action of recombinant host cells on the expressed polypeptide. Glutaminyl and asparaginyl residues are frequently post-translationally deamidated to the corresponding glutamyl and asparyl residues. Alternatively, these residues are deamidated under mildly acidic conditions. Other post-translational modifications include hydroxylation of proline and lysine, phosphorylation of hydroxyl groups of seryl or threonyl residues, methylation of the o-amino groups of lysine, arginine, and histidine side chains (T. E. Creighton, Proteins: Structure and Molecular Properties, W. H. Freeman & Co., San Francisco pp 79-86 [1983]), acetylation of the N-terminal amine and, in some instances, amidation of the C-terminal carboxyl.

It is understood that one way to define the variants and derivatives of the disclosed proteins herein is through defining the variants and derivatives in terms of homology/identity to specific known sequences. For example, SEQ ID NO:7 sets forth a particular sequence of gp120 (IIIa) and one of the disclosed Bob antigens is set forth in SEQ ID NO:1, for example. Specifically disclosed are variants of these and other proteins herein disclosed which have at least, 70% or 75% or 80% or 85% or 90% or 95% homology to the stated sequence. Those of skill in the art readily understand how to determine the homology of two proteins. For example, the homology can be calculated after aligning the two sequences so that the homology is at its highest level.

Another way of calculating homology can be performed by published algorithms. Optimal alignment of sequences for comparison may be conducted by the local homology algorithm of Smith and Waterman Adv. Appl. Math. 2: 482 (1981), by the homology alignment algorithm of Needleman and Wunsch, J. MoL Biol. 48: 443 (1970), by the search for similarity method of Pearson and Lipman, Proc. Natl. Acad. Sci. U.S.A. 85: 2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by inspection.

The same types of homology can be obtained for nucleic acids by for example the algorithms disclosed in Zuker, M. Science 244:48-52, 1989, Jaeger et al. Proc. Natl. Acad. Sci. USA86:7706-7710,1989, Jaeger et al. Methods Enzymol. 183:281-306, 1989 which are herein incorporated by reference for at least material related to nucleic acid alignment.

It is understood that the description of conservative mutations and homology can be combined together in any combination, such as embodiments that have at least 70% homology to a particular sequence wherein the variants are conservative mutations.

As this specification discusses various proteins and protein sequences it is understood that the nucleic acids that can encode those protein sequences are also disclosed. This would include all degenerate sequences related to a specific protein sequence, i.e. all nucleic acids having a sequence that encodes one particular protein sequence as well as all nucleic acids, including degenerate nucleic acids, encoding the disclosed variants and derivatives of the protein sequences. Thus, while each particular nucleic acid sequence may not be written out herein, it is understood that each and every sequence is in fact disclosed and described herein through the disclosed protein sequence. In addition, for example, a disclosed conservative derivative of SEQ ID NO:1 is shown in SEQ ID NO: 8, where the valine (V) at position 13 is changed to an isoleucine (I). It is understood that for this mutation all of the nucleic acid sequences that encode this particular derivative of the Bob antigen are also disclosed. It is also understood that while no amino acid sequence indicates what particular DNA sequence encodes that protein within an organism, where particular variants of a disclosed protein are disclosed herein, the known nucleic acid sequence that encodes that protein in the particular Bob antigen from which that antigen arises is also known and herein disclosed and described.

(2) Sequence Similarities

It is understood that as discussed herein the use of the terms homology and identity mean the same thing as similarity. Thus, for example, if the use of the word homology is used between two non-natural sequences it is understood that this is not necessarily indicating an evolutionary relationship between these two sequences, but rather is looking at the similarity or relatedness between their nucleic acid sequences. Many of the methods for determining homology between two evolutionarily related molecules are routinely applied to any two or more nucleic acids or proteins for the purpose of measuring sequence similarity regardless of whether they are evolutionarily related or not.

In general, it is understood that one way to define any known variants and derivatives or those that might arise, of the disclosed genes and proteins herein, is through defining the variants and derivatives in terms of homology to specific known sequences. This identity of particular sequences disclosed herein is also discussed elsewhere herein. In general, variants of genes and proteins herein disclosed typically have at least, about 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99 percent homology to the stated sequence or the native sequence. Those of skill in the art readily understand how to determine the homology of two proteins or nucleic acids, such as genes. For example, the homology can be calculated after aligning the two sequences so that the homology is at its highest level.

Another way of calculating homology can be performed by published algorithms. Optimal alignment of sequences for comparison may be conducted by the local homology algorithm of Smith and Waterman Adv. Appl. Math. 2: 482 (1981), by the homology alignment algorithm of Needleman and Wunsch, J. Mol. Biol. 48: 443 (1970), by the search for similarity method of Pearson and Lipman, Proc. Natl. Acad. Sci. U.S.A. 85: 2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by inspection.

The same types of homology can be obtained for nucleic acids by for example the algorithms disclosed in Zuker, M. Science 244:48-52, 1989, Jaeger et al. Proc. Natl. Acad. Sci. USA 86:7706-7710, 1989, Jaeger et al. Methods Enzymol. 183:281-306, 1989 which are herein incorporated by reference for at least material related to nucleic acid alignment. It is understood that any of the methods typically can be used and that in certain instances the results of these various methods may differ, but the skilled artisan understands if identity is found with at least one of these methods, the sequences would be said to have the stated identity, and be disclosed herein.

For example, as used herein, a sequence recited as having a particular percent homology to another sequence refers to sequences that have the recited homology as calculated by any one or more of the calculation methods described above. For example, a first sequence has 80 percent homology, as defined herein, to a second sequence if the first sequence is calculated to have 80 percent homology to the second sequence using the Zuker calculation method even if the first sequence does not have 80 percent homology to the second sequence as calculated by any of the other calculation methods. As another example, a first sequence has 80 percent homology, as defined herein, to a second sequence if the first sequence is calculated to have 80 percent homology to the second sequence using both the Zuker calculation method and the Pearson and Lipman calculation method even if the first sequence does not have 80 percent homology to the second sequence as calculated by the Smith and Waterman calculation method, the Needleman and Wunsch calculation method, the Jaeger calculation methods, or any of the other calculation methods. As yet another example, a first sequence has 80 percent homology, as defined herein, to a second sequence if the first sequence is calculated to have 80 percent homology to the second sequence using each of calculation methods (although, in practice, the different calculation methods will often result in different calculated homology percentages).

(3) Hybridization/Selective Hybridization

The term hybridization typically means a sequence driven interaction between at least two nucleic acid molecules, such as a primer or a probe and a gene. Sequence driven interaction means an interaction that occurs between two nucleotides or nucleotide analogs or nucleotide derivatives in a nucleotide specific manner. For example, G interacting with C or A interacting with T are sequence driven interactions. Typically sequence driven interactions occur on the Watson-Crick face or Hoogsteen face of the nucleotide. The hybridization of two nucleic acids is affected by a number of conditions and parameters known to those of skill in the art. For example, the salt concentrations, pH, and temperature of the reaction all affect whether two nucleic acid molecules will hybridize.

Parameters for selective hybridization between two nucleic acid molecules are well known to those of skill in the art. For example, in some embodiments selective hybridization conditions can be defined as stringent hybridization conditions. For example, stringency of hybridization is controlled by both temperature and salt concentration of either or both of the hybridization and washing steps. For example, the conditions of hybridization to achieve selective hybridization may involve hybridization in high ionic strength solution (6× SSC or 6× SSPE) at a temperature that is about 12-25° C. below the Tm (the melting temperature at which half of the molecules dissociate from their hybridization partners) followed by washing at a combination of temperature and salt concentration chosen so that the washing temperature is about 5° C. to 20° C. below the Tm. The temperature and salt conditions are readily determined empirically in preliminary experiments in which samples of reference DNA immobilized on filters are hybridized to a labeled nucleic acid of interest and then washed under conditions of different stringencies. Hybridization temperatures are typically higher for DNA-RNA and RNA-RNA hybridizations. The conditions can be used as described above to achieve stringency, or as is known in the art. (Sambrook et al., Molecular Cloning: A Laboratory Manual, 2nd Ed., Cold Spring Harbor Laboratory, Cold Spring Harbor, N.Y., 1989; Kunkel et al. Methods Enzymol. 1987:154:367, 1987 which is herein incorporated by reference for material at least related to hybridization of nucleic acids). A preferable stringent hybridization condition for a DNA:DNA hybridization can be at about 68° C. (in aqueous solution) in 6× SSC or 6× SSPE followed by washing at 68° C. Stringency of hybridization and washing, if desired, can be reduced accordingly as the degree of complementarity desired is decreased, and further, depending upon the G-C or A-T richness of any area wherein variability is searched for. Likewise, stringency of hybridization and washing, if desired, can be increased accordingly as homology desired is increased, and further, depending upon the G-C or A-T richness of any area wherein high homology is desired, all as known in the art.

Another way to define selective hybridization is by looking at the amount (percentage) of one of the nucleic acids bound to the other nucleic acid. For example, in some embodiments selective hybridization conditions would be when at least about, 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 percent of the limiting nucleic acid is bound to the non-limiting nucleic acid. Typically, the non-limiting primer is in for example, 10 or 100 or 1000 fold excess. This type of assay can be performed at under conditions where both the limiting and non-limiting primer are for example, 10 fold or 100 fold or 1000 fold below their k_{d}, or where only one of the nucleic acid molecules is 10 fold or 100 fold or 1000 fold or where one or both nucleic acid molecules are above their k_{d}.

Another way to define selective hybridization is by looking at the percentage of primer that gets enzymatically manipulated under conditions where hybridization is required to promote the desired enzymatic manipulation. For example, in some embodiments selective hybridization conditions would be when at least about, 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 percent of the primer is enzymatically manipulated under conditions which promote the enzymatic manipulation, for example if the enzymatic manipulation is DNA extension, then selective hybridization conditions would be when at least about 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 percent of the primer molecules are extended. Preferred conditions also include those suggested by the manufacturer or indicated in the art as being appropriate for the enzyme performing the manipulation.

Just as with homology, it is understood that there are a variety of methods herein disclosed for determining the level of hybridization between two nucleic acid molecules. It is understood that these methods and conditions may provide different percentages of hybridization between two nucleic acid molecules, but unless otherwise indicated meeting the parameters of any of the methods would be sufficient. For example if 80% hybridization was required and as long as hybridization occurs within the required parameters in any one of these methods it is considered disclosed herein.

It is understood that those of skill in the art understand that if a composition or method meets any one of these criteria for determining hybridization either collectively or singly it is a composition or method that is disclosed herein.

(4) Nucleic Acids

There are a variety of molecules disclosed herein that are nucleic acid based, including for example the nucleic acids that encode, for example the peptides set forth in SEQ ID NOs:1-6, as well as various functional nucleic acids. The disclosed nucleic acids are made up of for example, nucleotides, nucleotide analogs, or nucleotide substitutes. Non-limiting examples of these and other molecules are discussed herein. It is understood that for example, when a vector is expressed in a cell, that the expressed mRNA will typically be made up of A, C, G, and U. Likewise, it is understood that if, for example, an antisense molecule is introduced into a cell or cell environment through for example exogenous delivery, it is advantagous that the antisense molecule be made up of nucleotide analogs that reduce the degradation of the antisense molecule in the cellular environment.

(a) Nucleotides and Related Molecules

A nucleotide is a molecule that contains a base moiety, a sugar moiety and a phosphate moiety. Nucleotides can be linked together through their phosphate moieties and sugar moieties creating an internucleoside linkage. The base moiety of a nucleotide can be adenin-9-yl (A), cytosin-1-yl (C), guanin-9-yl (G), uracil-1-yl (U), and thymin-1-yl (T). The sugar moiety of a nucleotide is a ribose or a deoxyribose. The phosphate moiety of a nucleotide is pentavalent phosphate. An non-limiting example of a nucleotide would be 3′-AMP (3′-adenosine monophosphate) or 5′-GMP (5′-guanosine monophosphate).

A nucleotide analog is a nucleotide which contains some type of modification to either the base, sugar, or phosphate moieties. Modifications to the base moiety would include natural and synthetic modifications of A, C, G, and T/U as well as different purine or pyrimidine bases, such as uracil-5-yl (.psi.), hypoxanthin-9-yl (I), and 2-aminoadenin-9-yl. A modified base includes but is not limited to 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 8-azaguanine and 8-azaadenine, 7-deazaguanine and 7-deazaadenine and 3-deazaguanine and 3-deazaadenine. Additional base modifications can be found for example in U.S. Pat. No. 3,687,808, Englisch et al., Angewandte Chemie, International Edition, 1991, 30, 613, and Sanghvi, Y. S., Chapter 15, Antisense Research and Applications, pages 289-302, Crooke, S. T. and Lebleu, B. ed., CRC Press, 1993. Certain nucleotide analogs, such as 5-substituted pyrimidines, 6-azapyrimidines and N-2, N-6 and O-6 substituted purines, including 2-aminopropyladenine, 5-propynyluracil and 5-propynylcytosine. 5-methylcytosine can increase the stability of duplex formation. Often time base modifications can be combined with for example a sugar modifcation, such as 2′-O-methoxyethyl, to achieve unique properties such as increased duplex stability. There are numerous United States patents such as U.S. Pat. Nos. 4,845,205; 5,130,302; 5,134,066; 5,175,273; 5,367,066; 5,432,272; 5,457,187; 5,459,255; 5,484,908; 5,502,177; 5,525,711; 5,552,540; 5,587,469; 5,594,121, 5,596,091; 5,614,617; and 5,681,941, which detail and describe a range of base modifications. Each of these patents is herein incorporated by reference.

Nucleotide analogs can also include modifications of the sugar moiety. Modifications to the sugar moiety would include natural modifications of the ribose and deoxy ribose as well as synthetic modifications. Sugar modifications include but are not limited to the following modifications at the 2′ position: OH; F; O-, S-, or N-alkyl; O-, S-, or N-alkenyl; O-, S- or N-alkynyl; or O-alkyl-O-alkyl, wherein the alkyl, alkenyl and alkynyl may be substituted or unsubstituted C₁ to C₁₀, alkyl or C₂ to C₁₀ alkenyl and alkynyl. 2′ sugar modiifcations also include but are not limited to —O[(CH₂)ₙO]ₘCH₃, —O(CH₂)ₙOCH₃, —O(CH₂)ₙNH₂, —O(CH₂)ₙCH₃, —O(CH₂)ₙ—ONH₂, and —O(CH₂)ₙON[(CH₂)ₙ CH₃)]₂, where n and m are from 1 to about 10.

Other modifications at the 2′ position include but are not limted to: C₁ to C₁₀ lower alkyl, substituted lower alkyl, alkaryl, aralkyl, O-alkaryl or O-aralkyl, SH, SCH₃, OCN, Cl, Br, CN, CF₃, OCF₃, SOCH₃, SO₂ CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. Similar modifications may also be made at other positions on the sugar, particularly the 3′ position of the sugar on the 3′ terminal nucleotide or in 2′-5′ linked oligonucleotides and the 5′ position of 5′ terminal nucleotide. Modified sugars would also include those that contain modifications at the bridging ring oxygen, such as CH₂ and S. Nucleotide sugar analogs may also have sugar mimetics such as cyclobutyl moieties in place of the pentofuranosyl sugar. There are numerous United States patents that teach the preparation of such modified sugar structures such as U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; and 5,700,920, each of which is herein incorporated by reference in its entirety.

Nucleotide analogs can also be modified at the phosphate moiety. Modified phosphate moieties include but are not limited to those that can be modified so that the linkage between two nucleotides contains a phosphorothioate, chiral phosphorothioate, phosphorodithioate, phosphotriester, aminoalkylphosphotriester, methyl and other alkyl phosphonates including 3′-alkylene phosphonate and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates. It is understood that these phosphate or modified phosphate linkage between two nucleotides can be through a 3′-5′ linkage or a 2′-5′ linkage, and the linkage can contain inverted polarity such as 3′-5′ to 5′-3′ or 2′-5′ to 5′-2′. Various salts, mixed salts and free acid forms are also included. Numerous United States patents teach how to make and use nucleotides containing modified phosphates and include but are not limited to, U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; and 5,625,050, each of which is herein incorporated by reference.

It is understood that nucleotide analogs need only contain a single modification, but may also contain multiple modifications within one of the moieties or between different moieties.

Nucleotide substitutes are molecules having similar functional properties to nucleotides, but which do not contain a phosphate moiety, such as peptide nucleic acid (PNA). Nucleotide substitutes are molecules that will recognize nucleic acids in a Watson-Crick or Hoogsteen manner, but which are linked together through a moiety other than a phosphate moiety. Nucleotide substitutes are able to conform to a double helix type structure when interacting with the appropriate target nucleic acid.

Nucleotide substitutes are nucleotides or nucleotide analogs that have had the phosphate moiety and/or sugar moieties replaced. Nucleotide substitutes do not contain a standard phosphorus atom. Substitutes for the phosphate can be for example, short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH₂ component parts. Numerous United States patents disclose how to make and use these types of phosphate replacements and include but are not limited to U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; and 5,677,439, each of which is herein incorporated by reference.

It is also understood in a nucleotide substitute that both the sugar and the phosphate moieties of the nucleotide can be replaced, by for example an amide type linkage (aminoethylglycine) (PNA). U.S. Pat. Nos. 5,539,082; 5,714,331; and 5,719,262 teach how to make and use PNA molecules, each of which is herein incorporated by reference. (See also Nielsen et al., Science, 1991, 254, 1497-1500).

It is also possible to link other types of molecules (conjugates) to nucleotides or nucleotide analogs to enhance for example, cellular uptake. Conjugates can be chemically linked to the nucleotide or nucleotide analogs. Such conjugates include but are not limited to lipid moieties such as a cholesterol moiety (Letsinger et al., Proc. Natl. Acad. Sci. USA, 1989, 86, 6553-6556), cholic acid (Manoharan et al., Bioorg. Med. Chem. Let., 1994, 4, 1053-1060), a thioether, e.g., hexyl-S-tritylthiol (Manoharan et al., Ann. N.Y. Acad. Sci., 1992, 660, 306-309; Manoharan et al., Bioorg. Med. Chem. Let., 1993, 3, 2765-2770), a thiocholesterol (Oberhauser et al., Nucl. Acids Res., 1992, 20, 533-538), an aliphatic chain, e.g., dodecandiol or undecyl residues (Saison-Behmoaras et al., EMBO J., 1991, 10, 1111-1118; Kabanov et al., FEBS Lett., 1990, 259, 327-330; Svinarchuk et al., Biochimie, 1993, 75, 49-54), a phospholipid, e.g., di-hexadecyl-rac-glycerol or triethylammonium 1,2-di-O-hexadecyl-rac-glycero-3-H-phosphonate (Manoharan et al., Tetrahedron Lett., 1995, 36, 3651-3654; Shea et al., Nucl. Acids Res., 1990, 18, 3777-3783), a polyamine or a polyethylene glycol chain (Manoharan et al., Nucleosides & Nucleotides, 1995, 14, 969-973), or adamantane acetic acid (Manoharan et al., Tetrahedron Lett., 1995, 36, 3651-3654), a palmityl moiety (Mishra et al., Biochim. Biophys. Acta, 1995, 1264, 229-237), or an octadecylamine or hexylamino-carbonyl-oxycholesterol moiety (Crooke et al., J. Pharmacol. Exp. Ther., 1996, 277, 923-937. Numerous United States patents teach the preparation of such conjugates and include, but are not limited to U.S. Pat. Nos. 4,828,979; 4,948,882; 5,218,105; 5,525,465; 5,541,313; 5,545,730; 5,552,538; 5,578,717, 5,580,731; 5,591,584; 5,109,124; 5,118,802; 5,138,045; 5,414,077; 5,486,603; 5,512,439; 5,578,718; 5,608,046; 4,587,044; 4,605,735; 4,667,025; 4,762,779; 4,789,737; 4,824,941; 4,835,263; 4,876,335; 4,904,582; 4,958,013; 5,082,830; 5,112,963; 5,214,136; 5,082,830; 5,112,963; 5,214,136; 5,245,022; 5,254,469; 5,258,506; 5,262,536; 5,272,250; 5,292,873; 5,317,098; 5,371,241, 5,391,723; 5,416,203, 5,451,463; 5,510,475; 5,512,667; 5,514,785; 5,565,552; 5,567,810; 5,574,142; 5,585,481; 5,587,371; 5,595,726; 5,597,696; 5,599,923; 5,599,928 and 5,688,941, each of which is herein incorporated by reference.

A Watson-Crick interaction is at least one interaction with the Watson-Crick face of a nucleotide, nucleotide analog, or nucleotide substitute. The Watson-Crick face of a nucleotide, nucleotide analog, or nucleotide substitute includes the C2, N1, and C6 positions of a purine based nucleotide, nucleotide analog, or nucleotide substitute and the C2, N3, C4 positions of a pyrimidine based nucleotide, nucleotide analog, or nucleotide substitute.

A Hoogsteen interaction is the interaction that takes place on the Hoogsteen face of a nucleotide or nucleotide analog, which is exposed in the major groove of duplex DNA. The Hoogsteen face includes the N7 position and reactive groups (NH2 or O) at the C6 position of purine nucleotides.

(b) Sequences

There are a variety of sequences related to the gp120 gene and the Bob gene. These sequences and others are herein incorporated by reference in their entireties as well as for individual subsequences contained therein.

One particular sequence set forth in SEQ ID NO:9 is used herein, as an example, to exemplify the disclosed compositions and methods. It is understood that the description related to this sequence is applicable to any sequence related to SEQ ID NO:9 unless specifically indicated otherwise. Those of skill in the art understand how to resolve sequence discrepancies and differences and to adjust the compositions and methods relating to a particular sequence to other related sequences (i.e. sequences of gp120). Primers and/or probes can be designed for any disclosed sequence given the information disclosed herein and known in the art.

(c) Primers and Probes

Disclosed are compositions including primers and probes, which are capable of interacting with, for example, the Bob gene as disclosed herein. In certain embodiments the primers are used to support DNA amplification reactions. Typically the primers will be capable of being extended in a sequence specific manner. Extension of a primer in a sequence specific manner includes any methods wherein the sequence and/or composition of the nucleic acid molecule to which the primer is hybridized or otherwise associated directs or influences the composition or sequence of the product produced by the extension of the primer. Extension of the primer in a sequence specific manner therefore includes, but is not limited to, PCR, DNA sequencing, DNA extension, DNA polymerization, RNA transcription, or reverse transcription. Techniques and conditions that amplify the primer in a sequence specific manner are preferred. In certain embodiments the primers are used for the DNA amplification reactions, such as PCR or direct sequencing. It is understood that in certain embodiments the primers can also be extended using non-enzymatic techniques, where for example, the nucleotides or oligonucleotides used to extend the primer are modified such that they will chemically react to extend the primer in a sequence specific manner. Typically the disclosed primers hybridize with, for example, the Bob gene or region of the Bob gene or they hybridize with the complement of the Bob gene or complement of a region of the Bob gene.

The size of the primers or probes for interaction with, for example, the Bob gene in certain embodiments can be any size that supports the desired enzymatic manipulation of the primer, such as DNA amplification or the simple hybridization of the probe or primer. A typical Bob or other disclosed primer or probe would be at least 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 125, 150, 175, 200, 225, 250, 275, 300, 325, 350, 375, 400, 425, 450, 475, 500, 550, 600, 650, 700, 750, 800, 850, 900, 950, or 1000 nucleotides long.

In other embodiments a Bob or other disclosed primer or probe can be less than or equal to 6, 7, 8, 9, 10, 11, 12 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 125, 150, 175, 200, 225, 250, 275, 300, 325, 350, 375, 400, 425, 450, 475, 500, 550, 600, 650, 700, 750, 800, 850, 900, 950, or 1000 nucleotides long.

The primers for the Bob gene or other disclosed nucleic acid typically will be used to produce an amplified DNA product that contains fragments of the gene or disclosed nucleic acid, such as the regions of the Bob gene defined by SEQ ID NOs:11. In general, typically the size of the product will be such that the size can be accurately determined to within 3, or 2 or 1 nucleotides.

In certain embodiments this product is at least 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 125, 150, 175, 200, 225, 250, 275, 300, 325, 350, 375, 400, 425, 450, 475, 500, 550, 600, 650, 700, 750, 800, 850, 900, 950, or 1000 nucleotides long.

In other embodiments the product is less than or equal to 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 125, 150, 175, 200, 225, 250, 275, 300, 325, 350, 375, 400, 425, 450, 475, 500, 550, 600, 650, 700, 750, 800, 850, 900, 950, or 1000 nucleotides long.

(5) Delivery of the Compositions to Cells

There are a number of compositions and methods which can be used to deliver nucleic acids to cells, either in vitro or in vivo. These methods and compositions can largely be broken down into two classes: viral based delivery systems and non-viral based delivery systems. For example, the nucleic acids can be delivered through a number of direct delivery systems such as, electroporation, lipofection, calcium phosphate precipitation, plasmids, viral vectors, viral nucleic acids, phage nucleic acids, phages, cosmids, or via transfer of genetic material in cells or carriers such as cationic liposomes. Appropriate means for transfection, including viral vectors, chemical transfectants, or physico-mechanical methods such as electroporation and direct diffusion of DNA, are described by, for example, Wolff, J. A., et al., Science, 247, 1465-1468, (1990); and Wolff, J. A. Nature, 352, 815-818, (1991)Such methods are well known in the art and readily adaptable for use with the compositions and methods described herein. In certain cases, the methods will be modifed to specifically function with large DNA molecules. Further, these methods can be used to target certain diseases and cell populations by using the targeting characteristics of the carrier.

(a) Nucleic Acid Based Delivery Systems

Transfer vectors can be any nucleotide construction used to deliver genes into cells (e.g., a plasmid), or as part of a general strategy to deliver genes, e.g., as part of recombinant retrovirus or adenovirus (Ram et al. Cancer Res. 53:83-88, (1993)).

As used herein, plasmid or viral vectors are agents that transport the disclosed nucleic acids, such as vectors that encode molecules that interact with Bob and inhibit the interactions between gp120 and Bob into the cell without degradation and include a promoter yielding expression of the gene in the cells into which it is delivered. In some embodiments the delivery vehicles are derived from either a virus or a retrovirus. Viral vectors are, for example, Adenovirus, Adeno-associated virus, Herpes virus, Vaccinia virus, Polio virus, AIDS virus, neuronal trophic virus, Sindbis and other RNA viruses, including these viruses with the HIV backbone. Also preferred are any viral families which share the properties of these viruses which make them suitable for use as vectors. Retroviruses include Murine Maloney Leukemia virus, MMLV, and retroviruses that express the desirable properties of MMLV as a vector. Retroviral vectors are able to carry a larger genetic payload, i.e., a transgene or marker gene, than other viral vectors, and for this reason are a commonly used vector. However, they are not as useful in non-proliferating cells. Adenovirus vectors are relatively stable and easy to work with, have high titers, and can be delivered in aerosol formulation, and can transfect non-dividing cells. Pox viral vectors are large and have several sites for inserting genes, they are thermostable and can be stored at room temperature. A preferred embodiment is a viral vector which has been engineered so as to suppress the immune response of the host organism, elicited by the viral antigens. Preferred vectors of this type will carry coding regions for Interleukin 8 or 10.

Viral vectors can have higher transaction (ability to introduce genes) abilities than chemical or physical methods to introduce genes into cells. Typically, viral vectors contain, nonstructural early genes, structural late genes, an RNA polymerase III transcript, inverted terminal repeats necessary for replication and encapsidation, and promoters to control the transcription and replication of the viral genome. When engineered as vectors, viruses typically have one or more of the early genes removed and a gene or gene/promotor cassette is inserted into the viral genome in place of the removed viral DNA. Constructs of this type can carry up to about 8 kb of foreign genetic material. The necessary functions of the removed early genes are typically supplied by cell lines which have been engineered to express the gene products of the early genes in trans.

(i) Retroviral Vectors

A retrovirus is an animal virus belonging to the virus family of Retroviridae, including any types, subfamilies, genus, or tropisms. Retroviral vectors, in general, are described by Verma, I. M., Retroviral vectors for gene transfer. In Microbiology-1985, American Society for Microbiology, pp. 229-232, Washington, (1985), which is incorporated by reference herein. Examples of methods for using retroviral vectors for gene therapy are described in U.S. Pat. Nos. 4,868,116 and 4,980,286; PCT applications WO 90/02806 and WO 89/07136; and Mulligan, (Science 260:926-932 (1993)); the teachings of which are incorporated herein by reference.

A retrovirus is essentially a package which has packed into it nucleic acid cargo. The nucleic acid cargo carries with it a packaging signal, which ensures that the replicated daughter molecules will be efficiently packaged within the package coat. In addition to the package signal, there are a number of molecules which are needed in cis, for the replication, and packaging of the replicated virus. Typically a retroviral genome, contains the gag, pol, and env genes which are involved in the making of the protein coat. It is the gag, pol, and env genes which are typically replaced by the foreign DNA that it is to be transferred to the target cell. Retrovirus vectors typically contain a packaging signal for incorporation into the package coat, a sequence which signals the start of the gag transcription unit, elements necessary for reverse transcription, including a primer binding site to bind the tRNA primer of reverse transcription, terminal repeat sequences that guide the switch of RNA strands during DNA synthesis, a purine rich sequence 5′ to the 3′ LTR that serve as the priming site for the synthesis of the second strand of DNA synthesis, and specific sequences near the ends of the LTRs that enable the insertion of the DNA state of the retrovirus to insert into the host genome. The removal of the gag, pol, and env genes allows for about 8 kb of foreign sequence to be inserted into the viral genome, become reverse transcribed, and upon replication be packaged into a new retroviral particle. This amount of nucleic acid is sufficient for the delivery of a one to many genes depending on the size of each transcript. It is preferable to include either positive or negative selectable markers along with other genes in the insert.

Since the replication machinery and packaging proteins in most retroviral vectors have been removed (gag, pol, and env), the vectors are typically generated by placing them into a packaging cell line. A packaging cell line is a cell line which has been transfected or transformed with a retrovirus that contains the replication and packaging machinery, but lacks any packaging signal. When the vector carrying the DNA of choice is transfected into these cell lines, the vector containing the gene of interest is replicated and packaged into new retroviral particles, by the machinery provided in cis by the helper cell. The genomes for the machinery are not packaged because they lack the necessary signals.

(ii) Adenoviral Vectors

The construction of replication-defective adenoviruses has been described (Berkner et al., J. Virology 61:1213-1220 (1987); Massie et al., Mol. Cell. Biol. 6:2872-2883 (1986); Haj-Ahmad et al., J. Virology 57:267-274 (1986); Davidson et al., J. Virology 61:1226-1239 (1987); Zhang “Generation and identification of recombinant adenovirus by liposome-mediated transfection and PCR analysis” BioTechniques 15:868-872 (1993)). The benefit of the use of these viruses as vectors is that they are limited in the extent to which they can spread to other cell types, since they can replicate within an initial infected cell, but are unable to form new infectious viral particles. Recombinant adenoviruses have been shown to achieve high efficiency gene transfer after direct, in vivo delivery to airway epithelium, hepatocytes, vascular endothelium, CNS parenchyma and a number of other tissue sites (Morsy, J. Clin. Invest. 92:1580-1586 (1993); Kirshenbaum, J. Clin. Invest. 92:381-387 (1993); Roessler, J. Clin. Invest. 92:1085-1092 (1993); Moullier, Nature Genetics 4:154-159 (1993); La Salle, Science 259:988-990 (1993); Gomez-Foix, J. Biol. Chem. 267:25129-25134 (1992); Rich, Human Gene Therapy 4:461-476 (1993); Zabner, Nature Genetics 6:75-83 (1994); Guzman, Circulation Research 73:1201-1207 (1993); Bout, Human Gene Therapy 5:3-10 (1994); Zabner, Cell 75:207-216 (1993); Caillaud, Eur. J. Neuroscience 5:1287-1291 (1993); and Ragot, J. Gen. Virology 74:501-507 (1993)). Recombinant adenoviruses achieve gene transduction by binding to specific cell surface receptors, after which the virus is internalized by receptor-mediated endocytosis, in the same manner as wild type or replication-defective adenovirus (Chardonnet and Dales, Virology 40:462-477 (1970); Brown and Burlingham, J. Virology 12:386-396 (1973); Svensson and Persson, J. Virology 55:442-449 (1985); Seth, et al., J. Virol. 51:650-655 (1984); Seth, et al., Mol. Cell. Biol. 4:1528-1533 (1984); Varga et al., J. Virology 65:6061-6070 (1991); Wickham et al., Cell 73:309-319 (1993)).

A viral vector can be one based on an adenovirus which has had the E1 gene removed and these virons are generated in a cell line such as the human 293 cell line. In another preferred embodiment both the E1 and E3 genes are removed from the adenovirus genome.

(iii) Adeno-Asscociated Viral Vectors

Another type of viral vector is based on an adeno-associated virus (AAV). This defective parvovirus is a preferred vector because it can infect many cell types and is nonpathogenic to humans. AAV type vectors can transport about 4 to 5 kb and wild type AAV is known to stably insert into chromosome 19. Vectors which contain this site specific integration property are preferred. An especially preferred embodiment of this type of vector is the P4.1 C vector produced by Avigen, San Francisco, Calif., which can contain the herpes simplex virus thymidine kinase gene, HSV-tk, and/or a marker gene, such as the gene encoding the green fluorescent protein, GFP.

In another type of AAV virus, the AAV contains a pair of inverted terminal repeats (ITRs) which flank at least one cassette containing a promoter which directs cell-specific expression operably linked to a heterologous gene. Heterologous in this context refers to any nucleotide sequence or gene which is not native to the AAV or B19 parvovirus.

Typically the AAV and B19 coding regions have been deleted, resulting in a safe, noncytotoxic vector. The AAV ITRs, or modifications thereof, confer infectivity and site-specific integration, but not cytotoxicity, and the promoter directs cell-specific expression. U.S. Pat. No. 6,261,834 is herein incorproated by reference for material related to the AAV vector.

The disclosed vectors thus provide DNA molecules which are capable of integration into a mammalian chromosome without substantial toxicity.

The inserted genes in viral and retroviral usually contain promoters, and/or enhancers to help control the expression of the desired gene product. A promoter is generally a sequence or sequences of DNA that function when in a relatively fixed location in regard to the transcription start site. A promoter contains core elements required for basic interaction of RNA polymerase and transcription factors, and may contain upstream elements and response elements.

(iv) Large Payload Viral Vectors

Molecular genetic experiments with large human herpesviruses have provided a means whereby large heterologous DNA fragments can be cloned, propagated and established in cells permissive for infection with herpesviruses (Sun et al., Nature genetics 8: 33-41, 1994; Cotter and Robertson, Curr Opin Mol Ther 5: 633-644, 1999). These large DNA viruses (herpes simplex virus (HSV) and Epstein-Barr virus (EBV), have the potential to deliver fragments of human heterologous DNA>150 kb to specific cells. EBV recombinants can maintain large pieces of DNA in the infected B-cells as episomal DNA. Individual clones carried human genomic inserts up to 330 kb appeared genetically stable The maintenance of these episomes requires a specific EBV nuclear protein, EBNA1, constitutively expressed during infection with EBV. Additionally, these vectors can be used for transfection, where large amounts of protein can be generated transiently in vitro. Herpesvirus amplicon systems are also being used to package pieces of DNA>220 kb and to infect cells that can stably maintain DNA as episomes.

Other useful systems include, for example, replicating and host-restricted non-replicating vaccinia virus vectors.

(b) Non-Nucleic Acid Based Systems

The disclosed compositions can be delivered to the target cells in a variety of ways. For example, the compositions can be delivered through electroporation, or through lipofection, or through calcium phosphate precipitation. The delivery mechanism chosen will depend in part on the type of cell targeted and whether the delivery is occurring for example in vivo or in vitro.

Thus, the compositions can comprise, in addition to the disclosed compositions such as peptides or vectors for example, lipids such as liposomes, such as cationic liposomes (e.g., DOTMA, DOPE, DC-cholesterol) or anionic liposomes. Liposomes can further comprise proteins to facilitate targeting a particular cell, if desired. Administration of a composition comprising a compound and a cationic liposome can be administered to the blood afferent to a target organ or inhaled into the respiratory tract to target cells of the respiratory tract. Regarding liposomes, see, e.g., Brigham et al. Am. J. Resp. Cell. Mol. Biol. 1:95-100 (1989); Felgner et al. Proc. Natl. Acad. Sci USA 84:7413-7417 (1987); U.S. Pat. No. 4,897,355. Furthermore, the compound can be administered as a component of a microcapsule that can be targeted to specific cell types, such as macrophages, or where the diffusion of the compound or delivery of the compound from the microcapsule is designed for a specific rate or dosage.

In the methods described above which include the administration and uptake of exogenous DNA into the cells of a subject (i.e., gene transduction or transfection), delivery of the compositions to cells can be via a variety of mechanisms. As one example, delivery can be via a liposome, using commercially available liposome preparations such as LIPOFECTIN, LIPOFECTAMINE (GIBCO-BRL, Inc., Gaithersburg, Md.), SUPERFECT (Qiagen, Inc. Hilden, Germany) and TRANSFECTAM (Promega Biotec, Inc., Madison, Wis.), as well as other liposomes developed according to procedures standard in the art. In addition, the disclosed nucleic acids or vectors can be delivered in vivo by electroporation, the technology for which is available from Genetronics, Inc. (San Diego, Calif.) as well as by means of a SONOPORATION machine (ImaRx Pharmaceutical Corp., Tucson, Ariz.).

The materials may be in solution, suspension (for example, incorporated into microparticles, liposomes, or cells). These may be targeted to a particular cell type via antibodies, receptors, or receptor ligands. The following references are examples of the use of this technology to target specific proteins to tumor tissue (Senter, et al., Bioconiugate Chem., 2:447-451, (1991); Bagshawe, K. D., Br. J. Cancer, 60:275-281, (1989); Bagshawe, et al., Br. J. Cancer, 58:700-703, (1988); Senter, et al., Bioconjugate Chem., 4:3-9, (1993); Battelli, et al., Cancer Immunol. Immunother., 35:421-425, (1992); Pietersz and McKenzie, Immunolog. Reviews, 129:57-80, (1992); and Roffler, et al., Biochem. Pharmacol, 42:2062-2065, (1991)). These techniques can be used for a variety of other specific cell types. Vehicles such as “stealth” and other antibody conjugated liposomes (including lipid mediated drug targeting to colonic carcinoma), receptor mediated targeting of DNA through cell specific ligands, lymphocyte directed tumor targeting, and highly specific therapeutic retroviral targeting of murine glioma cells in vivo. The following references are examples of the use of this technology to target specific proteins to tumor tissue (Hughes et al., Cancer Research, 49:6214-6220, (1989); and Litzinger and Huang, Biochimica et Biophysica Acta, 1104:179-187, (1992)). In general, receptors are involved in pathways of endocytosis, either constitutive or ligand induced. These receptors cluster in clathrin-coated pits, enter the cell via clathrin-coated vesicles, pass through an acidified endosome in which the receptors are sorted, and then either recycle to the cell surface, become stored intracellularly, or are degraded in lysosomes. The internalization pathways serve a variety of functions, such as nutrient uptake, removal of activated proteins, clearance of macromolecules, opportunistic entry of viruses and toxins, dissociation and degradation of ligand, and receptor-level regulation. Many receptors follow more than one intracellular pathway, depending on the cell type, receptor concentration, type of ligand, ligand valency, and ligand concentration. Molecular and cellular mechanisms of receptor-mediated endocytosis has been reviewed (Brown and Greene, DNA and Cell Biology 10:6, 399-409 (1991)).

Nucleic acids that are delivered to cells which are to be integrated into the host cell genome, typically contain integration sequences. These sequences are often viral related sequences, particularly when viral based systems are used. These viral intergration systems can also be incorporated into nucleic acids which are to be delivered using a non-nucleic acid based system of deliver, such as a liposome, so that the nucleic acid contained in the delivery system can be come integrated into the host genome.

Other general techniques for integration into the host genome include, for example, systems designed to promote homologous recombination with the host genome. These systems typically rely on sequence flanking the nucleic acid to be expressed that has enough homology with a target sequence within the host cell genome that recombination between the vector nucleic acid and the target nucleic acid takes place, causing the delivered nucleic acid to be integrated into the host genome. These systems and the methods necessary to promote homologous recombination are known to those of skill in the art.

(c) In Vivo/Ex Vivo

As described above, the compositions can be administered in a pharmaceutically acceptable carrier and can be delivered to the subject's cells in vivo and/or ex vivo by a variety of mechanisms well known in the art (e.g., uptake of naked DNA, liposome fusion, intramuscular injection of DNA via a gene gun, endocytosis and the like).

If ex vivo methods are employed, cells or tissues can be removed and maintained outside the body according to standard protocols well known in the art. The compositions can be introduced into the cells via any gene transfer mechanism, such as, for example, calcium phosphate mediated gene delivery, electroporation, microinjection or proteoliposomes. The transduced cells can then be infused (e.g., in a pharmaceutically acceptable carrier) or homotopically transplanted back into the subject per standard methods for the cell or tissue type. Standard methods are known for transplantation or infusion of various cells into a subject.

(6) Expression Systems

The nucleic acids that are delivered to cells typically contain expression controlling systems. For example, the inserted genes in viral and retroviral systems usually contain promoters, and/or enhancers to help control the expression of the desired gene product. A promoter is generally a sequence or sequences of DNA that function when in a relatively fixed location in regard to the transcription start site. A promoter contains core elements required for basic interaction of RNA polymerase and transcription factors, and may contain upstream elements and response elements.

(a) Viral Promoters and Enhancers

Preferred promoters controlling transcription from vectors in mammalian host cells may be obtained from various sources, for example, the genomes of viruses such as: polyoma, Simian Virus 40 (SV40), adenovirus, retroviruses, hepatitis-B virus and most preferably cytomegalovirus, or from heterologous mammalian promoters, e.g. beta actin promoter. The early and late promoters of the SV40 virus are conveniently obtained as an SV40 restriction fragment which also contains the SV40 viral origin of replication (Fiers et al., Nature, 273: 113 (1978)). The immediate early promoter of the human cytomegalovirus is conveniently obtained as a HindIII E restriction fragment (Greenway, P. J. et al., Gene 18: 355-360 (1982)). Of course, promoters from the host cell or related species also are useful herein.

Enhancer generally refers to a sequence of DNA that functions at no fixed distance from the transcription start site and can be either 5′ (Laimins, L. et al., Proc. Natl. Acad. Sci. 78: 993 (1981)) or 3′ (Lusky, M. L., et al., Mol. Cell Bio. 3: 1108 (1983)) to the transcription unit. Furthermore, enhancers can be within an intron (Banerji, J. L. et al., Cell 33: 729 (1983)) as well as within the coding sequence itself (Osborne, T. F., et al., Mol. Cell Bio. 4: 1293 (1984)). They are usually between 10 and 300 bp in length, and they function in cis. Enhancers f unction to increase transcription from nearby promoters. Enhancers also often contain response elements that mediate the regulation of transcription. Promoters can also contain response elements that mediate the regulation of transcription. Enhancers often determine the regulation of expression of a gene. While many enhancer sequences are now known from mammalian genes (globin, elastase, albumin, -fetoprotein and insulin), typically one will use an enhancer from a eukaryotic cell virus for general expression. Preferred examples are the SV40 enhancer on the late side of the replication origin (bp 100-270), the cytomegalovirus early promoter enhancer, the polyoma enhancer on the late side of the replication origin, and adenovirus enhancers.

The promotor and/or enhancer may be specifically activated either by light or specific chemical events which trigger their function. Systems can be regulated by reagents such as tetracycline and dexamethasone. There are also ways to enhance viral vector gene expression by exposure to irradiation, such as gamma irradiation, or alkylating chemotherapy drugs.

In certain embodiments the promoter and/or enhancer region can act as a constitutive promoter and/or enhancer to maximize expression of the region of the transcription unit to be transcribed. In certain constructs the promoter and/or enhancer region be active in all eukaryotic cell types, even if it is only expressed in a particular type of cell at a particular time. A preferred promoter of this type is the CMV promoter (650 bases). Other preferred promoters are SV40 promoters, cytomegalovirus (full length promoter), and retroviral vector LTF.

It has been shown that all specific regulatory elements can be cloned and used to construct expression vectors that are selectively expressed in specific cell types such as melanoma cells. The glial fibrillary acetic protein (GFAP) promoter has been used to selectively express genes in cells of glial origin.

Expression vectors used in eukaryotic host cells (yeast, fungi, insect, plant, animal, human or nucleated cells) may also contain sequences necessary for the termination of transcription which may affect mRNA expression. These regions are transcribed as polyadenylated segments in the untranslated portion of the mRNA encoding tissue factor protein. The 3′ untranslated regions also include transcription termination sites. It is preferred that the transcription unit also contain a polyadenylation region. One benefit of this region is that it increases the likelihood that the transcribed unit will be processed and transported like mRNA. The identification and use of polyadenylation signals in expression constructs is well established. It is preferred that homologous polyadenylation signals be used in the transgene constructs. In certain transcription units, the polyadenylation region is derived from the SV40 early polyadenylation signal and consists of about 400 bases. It is also preferred that the transcribed units contain other standard sequences alone or in combination with the above sequences improve expression from, or stability of, the construct.

(b) Markers

The viral vectors can include nucleic acid sequence encoding a marker product. This marker product is used to determine if the gene has been delivered to the cell and once delivered is being expressed. Preferred marker genes are the E. Coli lacZ gene, which encodes β-galactosidase, and green fluorescent protein.

In some embodiments the marker may be a selectable marker. Examples of suitable selectable markers for mammalian cells are dihydrofolate reductase (DHFR), thymidine kinase, neomycin, neomycin analog G418, hydromycin, and puromycin. When such selectable markers are successfully transferred into a mammalian host cell, the transformed mammalian host cell can survive if placed under selective pressure. There are two widely used distinct categories of selective regimes. The first category is based on a cell's metabolism and the use of a mutant cell line which lacks the ability to grow independent of a supplemented media. Two examples are: CHO DHFR-cells and mouse LTK-cells. These cells lack the ability to grow without the addition of such nutrients as thymidine or hypoxanthine. Because these cells lack certain genes necessary for a complete nucleotide synthesis pathway, they cannot survive unless the missing nucleotides are provided in a supplemented media. An alternative to supplementing the media is to introduce an intact DHFR or TK gene into cells lacking the respective genes, thus altering their growth requirements. Individual cells which were not transformed with the DHFR or TK gene will not be capable of survival in non-supplemented media.

The second category is dominant selection which refers to a selection scheme used in any cell type and does not require the use of a mutant cell line. These schemes typically use a drug to arrest growth of a host cell. Those cells which have a novel gene would express a protein conveying drug resistance and would survive the selection. Examples of such dominant selection use the drugs neomycin, (Southern P. and Berg, P., J. Molec. Appl. Genet. 1: 327 (1982)), mycophenolic acid, (Mulligan, R. C. and Berg, P. Science 209: 1422 (1980)) or hygromycin, (Sugden, B. et al., Mol. Cell. Biol. 5: 410-413 (1985)). The three examples employ bacterial genes under eukaryotic control to convey resistance to the appropriate drug G418 or neomycin (geneticin), xgpt (mycophenolic acid) or hygromycin, respectively. Others include the neomycin analog G418 and puramycin.

(7) Pharmaceutical Carriers/Delivery of Pharmceutical Products

As described above, the compositions can also be administered in vivo in a pharmaceutically acceptable carrier. By “pharmaceutically acceptable” is meant a material that is not biologically or otherwise undesirable, i.e., the material may be administered to a subject, along with the nucleic acid or vector, without causing any undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art.

The compositions may be administered orally, parenterally (e.g., intravenously), by intramuscular injection, by intraperitoneal injection, transdermally, extracorporeally, topically or the like, although topical intranasal administration or administration by inhalant is typically preferred. As used herein, “topical intranasal administration” means delivery of the compositions into the nose and nasal passages through one or both of the nares and can comprise delivery by a spraying mechanism or droplet mechanism, or through aerosolization of the nucleic acid or vector. The latter may be effective when a large number of animals is to be treated simultaneously. Administration of the compositions by inhalant can be through the nose or mouth via delivery by a spraying or droplet mechanism. Delivery can also be directly to any area of the respiratory system (e.g., lungs) via intubation. The exact amount of the compositions required will vary from subject to subject, depending on the species, age, weight and general condition of the subject, the severity of the allergic disorder being treated, the particular nucleic acid or vector used, its mode of administration and the like. Thus, it is not possible to specify an exact amount for every composition. However, an appropriate amount can be determined by one of ordinary skill in the art using only routine experimentation given the teachings herein.

Parenteral administration of the composition, if used, is generally characterized by injection. Injectables can be prepared in conventional forms, either as liquid solutions or suspensions, solid forms suitable for solution of suspension in liquid prior to injection, or as emulsions. A more recently revised approach for parenteral administration involves use of a slow release or sustained release system such that a constant dosage is maintained. See, e.g., U.S. Pat. No. 3,610,795, which is incorporated by reference herein.

The materials may be in solution, suspension (for example, incorporated into microparticles, liposomes, or cells). These may be targeted to a particular cell type via antibodies, receptors, or receptor ligands. The following references are examples of the use of this technology to target specific proteins to tumor tissue (Senter, et al., Bioconiugate Chem., 2:447-451, (1991); Bagshawe, K. D., Br. J. Cancer, 60:275-281, (1989); Bagshawe, et al., Br. J. Cancer. 58:700-703, (1988); Senter, et al., Bioconiugate Chem., 4:3-9, (1993); Battelli, et al., Cancer Immunol. Immunother., 35:421-425, (1992); Pietersz and McKenzie, Immunolog. Reviews, 129:57-80, (1992); and Roffler, et al., Biochem. Pharmacol, 42:2062-2065, (1991)). Vehicles such as “stealth” and other antibody conjugated liposomes (including lipid mediated drug targeting to colonic carcinoma), receptor mediated targeting of DNA through cell specific ligands, lymphocyte directed tumor targeting, and highly specific therapeutic retroviral targeting of murine glioma cells in vivo. The following references are examples of the use of this technology to target specific proteins to tumor tissue (Hughes et al., Cancer Research, 49:6214-6220, (1989); and Litzinger and Huang, Biochimica et Biophysica Acta, 1104:179-187, (1992)). In general, receptors are involved in pathways of endocytosis, either constitutive or ligand induced. These receptors cluster in clathrin-coated pits, enter the cell via clathrin-coated vesicles, pass through an acidified endosome in which the receptors are sorted, and then either recycle to the cell surface, become stored intracellularly, or are degraded in lysosomes. The internalization pathways serve a variety of functions, such as nutrient uptake, removal of activated proteins, clearance of macromolecules, opportunistic entry of viruses and toxins, dissociation and degradation of ligand, and receptor-level regulation. Many receptors follow more than one intracellular pathway, depending on the cell type, receptor concentration, type of ligand, ligand valency, and ligand concentration. Molecular and cellular mechanisms of receptor-mediated endocytosis has been reviewed (Brown and Greene, DNA and Cell Biology 10:6, 399-409 (1991)).

(a) Pharmaceutically Acceptable Carriers

The compositions, including antibodies, can be used therapeutically in combination with a pharmaceutically acceptable carrier.

Pharmaceutical carriers are known to those skilled in the art. These most typically would be standard carriers for administration of drugs to humans, including solutions such as sterile water, saline, and buffered solutions at physiological pH. The compositions can be administered intramuscularly or subcutaneously. Other compounds will be administered according to standard procedures used by those skilled in the art.

Pharmaceutical compositions may include carriers, thickeners, diluents, buffers, preservatives, surface active agents and the like in addition to the molecule of choice. Pharmaceutical compositions may also include one or more active ingredients such as antimicrobial agents, antiinflammatory agents, anesthetics, and the like.

The pharmaceutical composition may be administered in a number of ways depending on whether local or systemic treatment is desired, and on the area to be treated. Administration may be topically (including ophthalmically, vaginally, rectally, intranasally), orally, by inhalation, or parenterally, for example by intravenous drip, subcutaneous, intraperitoneal or intramuscular injection. The disclosed antibodies can be administered intravenously, intraperitoneally, intramuscularly, subcutaneously, intracavity, or transdermally.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Formulations for topical administration may include ointments, lotions, creams, gels, drops, suppositories, sprays, liquids and powders. Conventional pharmaceutical carriers, aqueous, powder or oily bases, thickeners and the like may be necessary or desirable.

Compositions for oral administration include powders or granules, suspensions or solutions in water or non-aqueous media, capsules, sachets, or tablets. Thickeners, flavorings, diluents, emulsifiers, dispersing aids or binders may be desirable.

Some of the compositions may potentially be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, trialkyl and aryl amines and substituted ethanolamines.

(b) Therapeutic Uses

The dosage ranges for the administration of the compositions are those large enough to produce the desired effect in which the symptoms disorder are effected. The dosage should not be so large as to cause adverse side effects, such as unwanted cross-reactions, anaphylactic reactions, and the like. Generally, the dosage will vary with the age, condition, sex and extent of the disease in the patient and can be determined by one of skill in the art. The dosage can be adjusted by the individual physician in the event of any counterindications. Dosage can vary, and can be administered in one or more dose administrations daily, for one or several days.

The compositions can also be used for tracking changes within cellular chromosomes or for the delivery of diagnositc tools for example can be delivered in ways similar to those described for the pharmaceutical products.

The compositions can also be used for example as tools to isolate and test new drug candidates for a variety of diseases. They can also be used for the continued isolation and study, for example, the cell cycle. There use as exogenous DNA delivery devices can be expanded for nearly any reason desired by those of skill in the art.

(8) Chips and Micro Arrays

Disclosed are chips where at least one address is the sequences or part of the sequences set forth in any of the nucleic acid sequences disclosed herein. Also disclosed are chips where at least one address is the sequences or portion of sequences set forth in any of the peptide sequences disclosed herein.

Also disclosed are chips where at least one address is a variant of the sequences or part of the sequences set forth in any of the nucleic acid sequences disclosed herein. Also disclosed are chips where at least one address is a variant of the sequences or portion of sequences set forth in any of the peptide sequences disclosed herein.

(9) Computer Readable Mediums

It is understood that the disclosed nucleic acids and proteins can be represented as a sequence consisting of the nucleotides of amino acids. There are a variety of ways to display these sequences, for example the nucleotide guanosine can be represented by G or g. Likewise the amino acid valine can be represented by Val or V. Those of skill in the art understand how to display and express any nucleic acid or protein sequence in any of the variety of ways that exist, each of which is considered herein disclosed. Specifically contemplated herein is the display of these sequences on computer readable mediums, such as, commercially available floppy disks, tapes, chips, hard drives, compact disks, and video disks, or other computer readable mediums. Also disclosed are the binary code representations of the disclosed sequences. Those of skill in the art understand what computer readable mediums. Thus, computer readable mediums on which the nucleic acids or protein sequences are recorded, stored, or saved.

Disclosed are computer readable mediums comprising the sequences and information regarding the sequences set forth herein. Also disclosed are computer readable mediums comprising the sequences and information regarding the sequences set forth herein wherein the sequences do not include SEQ ID Nos:1-6.

(10) Kits

Disclosed herein are kits that are drawn to reagents that can be used in practicing the methods disclosed herein. The kits can include any reagent or combination of reagent discussed herein or that would be understood to be required or beneficial in the practice of the disclosed methods. For example, the kits could include primers to perform the amplification reactions discussed in certain embodiments of the methods, as well as the buffers and enzymes required to use the primers as intended. For example, disclosed is a kit for producing antibodies that bind SEQ ID NOs:1-6 comprising the oligonucleotides set forth in SEQ ID NOs:1-6.

(11) Compositions with Similar Funtions

It is understood that the compositions disclosed herein have certain functions, such as reducing gp120-Bob interactions or binding Bob. Disclosed herein are certain structural requirements for performing the disclosed functions, and it is understood that there are a variety of structures which can perform the same function which are related to the disclosed structures, and that these structures will ultimately achieve the same result, for example inhibition of gp120-Bob binding.

2. Methods of Making the Compositions

The compositions disclosed herein and the compositions necessary to perform the disclosed methods can be made using any method known to those of skill in the art for that particular reagent or compound unless otherwise specifically noted.

a) Nucleic Acid Synthesis

For example, the nucleic acids, such as, the oligonucleotides to be used as primers can be made using standard chemical synthesis methods or can be produced using enzymatic methods or any other known method. Such methods can range from standard enzymatic digestion followed by nucleotide fragment isolation (see for example, Sambrook et al., Molecular Cloning: A Laboratory Manual, 2nd Edition (Cold Spring Harbor Laboratory Press, Cold Spring Harbor, N.Y., 1989) Chapters 5, 6) to purely synthetic methods, for example, by the cyanoethyl phosphoramidite method using a Milligen or Beckman System 1Plus DNA synthesizer (for example, Model 8700 automated synthesizer of Milligen-Biosearch, Burlington, Mass. or ABI Model 380B). Synthetic methods useful for making oligonucleotides are also described by Ikuta et al., Ann. Rev. Biochem. 53:323-356 (1984), (phosphotriester and phosphite-triester methods), and Narang et al., Methods Enzymol., 65:610-620 (1980), (phosphotriester method). Protein nucleic acid molecules can be made using known methods such as those described by Nielsen et al., Bioconjug. Chem. 5:3-7 (1994).

b) Peptide Synthesis

One method of producing the disclosed proteins, such as SEQ ID NO:1-6, is to link two or more peptides or polypeptides together by protein chemistry techniques. For example, peptides or polypeptides can be chemically synthesized using currently available laboratory equipment using either Fmoc (9-fluorenylmethyloxycarbonyl) or Boc (tert-butyloxycarbonoyl) chemistry. (Applied Biosystems, Inc., Foster City, Calif.). One skilled in the art can readily appreciate that a peptide or polypeptide corresponding to the disclosed proteins, for example, can be synthesized by standard chemical reactions. For example, a peptide or polypeptide can be synthesized and not cleaved from its synthesis resin whereas the other fragment of a peptide or protein can be synthesized and subsequently cleaved from the resin, thereby exposing a terminal group which is functionally blocked on the other fragment. By peptide condensation reactions, these two fragments can be covalently joined via a peptide bond at their carboxyl and amino termini, respectively, to form an antibody, or fragment thereof (Grant G A (1992) Synthetic Peptides: A User Guide. W. H. Freeman and Co., N.Y. (1992); Bodansky M and Trost B., Ed. (1993) Principles of Peptide Synthesis. Springer-Verlag Inc., NY (which is herein incorporated by reference at least for material related to peptide synthesis). Alternatively, the peptide or polypeptide is independently synthesized in vivo as described herein. Once isolated, these independent peptides or polypeptides may be linked to form a peptide or fragment thereof via similar peptide condensation reactions.

For example, enzymatic ligation of cloned or synthetic peptide segments allow relatively short peptide fragments to be joined to produce larger peptide fragments, polypeptides or whole protein domains (Abrahmsen L et al., Biochemistry, 30:4151 (1991)). Alternatively, native chemical ligation of synthetic peptides can be utilized to synthetically construct large peptides or polypeptides from shorter peptide fragments. This method consists of a two step chemical reaction (Dawson et al. Synthesis of Proteins by Native Chemical Ligation. Science, 266:776-779 (1994)). The first step is the chemoselective reaction of an unprotected synthetic peptide—thioester with another unprotected peptide segment containing an amino-terminal Cys residue to give a thioester-linked intermediate as the initial covalent product. Without a change in the reaction conditions, this intermediate undergoes spontaneous, rapid intramolecular reaction to form a native peptide bond at the ligation site (Baggiolini M et al. (1992) FEBS Lett. 307:97-101; Clark-Lewis I et al., J. Biol. Chem., 269:16075 (1994); Clark-Lewis I et al., Biochemistry, 30:3128 (1991); Rajarathnam K et al., Biochemistry 33:6623-30 (1994)).

Alternatively, unprotected peptide segments are chemically linked where the bond formed between the peptide segments as a result of the chemical ligation is an unnatural (non-peptide) bond (Schnolzer, M et al. Science, 256:221 (1992)). This technique has been used to synthesize analogs of protein domains as well as large amounts of relatively pure proteins with full biological activity (deLisle Milton RC et al., Techniques in Protein Chemistry IV. Academic Press, New York, pp. 257-267 (1992)).

c) Process for Making the Compositions

Disclosed are processes for making the compositions as well as making the intermediates leading to the compositions. For example, disclosed are peptides in SEQ ID NOs:1-6. There are a variety of methods that can be used for making these compositions, such as synthetic chemical methods and standard molecular biology methods. It is understood that the methods of making these and the other disclosed compositions are specifically disclosed.

Disclosed are any of the disclosed peptides produced by the process of expressing any of the disclosed nucleic acids. Disclosed are any of the non-naturally occurring disclosed peptides produced by the process of expressing any of the disclosed nucleic acids. Disclosed are any of the disclosed peptides produced by the process of expressing any of the non-naturally disclosed nucleic acids.

Disclosed are animals produced by the process of transfecting a cell within the animal with any of the nucleic acid molecules disclosed herein. Disclosed are animals produced by the process of transfecting a cell within the animal any of the nucleic acid molecules disclosed herein, wherein the animal is a mammal. Also disclosed are animals produced by the process of transfecting a cell within the animal any of the nucleic acid molecules disclosed herein, wherein the mammal is mouse, rat, rabbit, cow, sheep, pig, or primate.

Also disclosed are animals produced by the process of adding to the animal any of the cells disclosed herein.

3. Methods of Using the Compositions

a) Methods of Using the Compositions as Research Tools

The disclosed compositions can be used in a variety of ways as research tools. For example, the disclosed compositions, such as SEQ ID NOs:1-6 can be used to study the interactions between gp120 and Bob, by for example acting as inhibitors of binding.

The compositions can be used for example as targets in combinatorial chemistry protocols or other screening protocols to isolate molecules that possess desired functional properties related to the polyclonal antibodies disclosed herein.

The disclosed compositions can be used as discussed herein as either reagents in micro arrays or as reagents to probe or analyze existing microarrays. The disclosed compositions can be used in any known method for isolating or identifying single nucleotide polymorphisms. The compositions can also be used in any method for determining allelic analysis. The compositions can also be used in any known method of screening assays, related to chip/micro arrays. The compositions can also be used in any known way of using the computer readable embodiments of the disclosed compositions, for example, to study relatedness or to perform molecular modeling analysis related to the disclosed compositions.

These results indicate that the use of the disclosed compositions, such as Bob37, could be used in a preventative way, with for example, other therapies. For example, someone at risk for acquiring an HIV infection, could take a disclosed composition to reduce the chance for initial infection.

b) Methods of Targeting HIV Related Diseases

The disclosed compositions and methods can be used to target or alleviate HIV-related diseases or symptoms which are related to Bob activation as discussed herein. For example, progressive loss of CD4 lymphocytes, that can lead to immunodeficiency and AIDS (via enhanced infectivity of otherwise unactivated CD4-positive T cells or enhances apoptosis, or of effects on macrophages). Also HIV enteropathy, HIV nephropathy, HIV-related hyperlipidemia, and HIV-related infertility and loss of testicular germ cells are examples of HIV related disorders and/or symptoms that can be addressed by the disclosed compositions, for example, compositions that prevent the interaction between gp120 and Bob.

### C. EXAMPLES

Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### 1. Example 1

### gp120 Interacts with Bob

a) Materials and Methods

(1) Primary Antibodies

Two sets of three peptide antigens from the human GPR15/Bob sequence were synthesized, namely HAEDFARRRKRSVSL (SEQ ID NO:1) the carboxy end of the carboxy terminal intracellular domain, DKEASLGLWRTGSFLCK (SEQ ID NO:2) the N terminal end of the first extracellular loop, and MDPEETSVYLDYYYATS (SEQ ID NO:3) the N terminal end of the extracellular domain; and SGLRQEHYLPSAILQ (SEQ ID NO:4) from the third extracellular loop, RELTLIDDKPYCAEKKAT (SEQ ID NO:5) from the second extracellular loop, and KNYDFGSSTETSDSHLTK (SEQ ID NO:6) from the carboxy terminal intracellular domain. Rabbits were injected with either the first three or second three peptides, and immune sera were affinity purified with the corresponding antigen peptides. These antibodies were named anti-Bob37 and anti-Bob39 respectively. This work was performed at Research Genetics, Huntsville Ala. Mouse monoclonal anti-acetylated tubulin (5 μg/ml, Sigma), monoclonal antiCXCR4 and antiCCR5 (5 μg/ml, AIDS Research and Reference Reagent Program), polyclonal antigalactosyl ceramide (Sigma), and monoclonal anti galactosyl ceramide (Chemicon) were also used.

MDPEET . . . , DKEAS . . . , and KNYDF . . . were used together, both as antigens and for affinity purification. One rabbit, whose antibodies were used, developed high titer antibodies to MDPEET . . . and DKEAS . . . but not KNYDF. These antibodies were designated antiBob37. RELTL . . . , SGLRQ . . . , and HAEDF . . . were coimmunized into rabbits. In one rabbit, all three peptides engendered high titer antibodies, which were affinity purified. These antibodies were designated antiBob39.

(2) Western Blotting

Western blots were made from tissue or cultured cells; the soluble fractions of homogenates were prepared in TritonX100-containing lysis buffer²⁵ at 4° C. Electrophoresis in 10% polyacrilamide gels was done with 50 micrograms protein per lane, and the protein was electroblotted onto nitrocellulose. The blots were blocked with 20 mM Tris (pH 7.6), 140 mM NaCl, 0.1% Tween 20, and 5 g/100 ml nonfat dry milk for one hour, then treated with 0.5 μg/ml primary antibody in the same buffer for one hour. Blots were visualized either by chemiluminescence using ECL reagents (Amersham/Pharmacia) or by alkaline phosphatase conjugated secondary antibodies with NBT/BCIP staining. Negative controls included omitting the primary antibody and overnight preabsorption of the primary antibody with 500-fold excess of the three appropriate peptides.

The HT-29 lysates were immunoprecipitated with 2 μg/ml Bob37 or polyclonal antigalactosyl ceramide (1:100 dilution), then incubated with protein A/G Sepharose (1.5% v/v) for 2 hours (both steps on a rocker at 4° C.). The Sepharose beads were washed, then boiled and used in Western blotting. These Western blots were stained with either anti-Bob37, anti-Bob39, or monoclonal antigalactosyl ceramide antibodies at 0.5 μg/ml.

(3) Immunoprecipitation

Immunoprecipitation of a 36 kD protein corresponding to Bob was obtained with 2 micrograms/ml of either the antiBob37 or the antiBob39 antibody for 2 hours, then a 2 hour incubation with 1.5% v/v protein A/G Sepharose (both steps at 0-4° C.). The beads were then washed, then boiled and used in Western blots as above.

### (4) RNA in Situ Hybridization

RNA in situ hybridization was done with a digoxygenin labeled cRNA antisense riboprobe prepared as follows: PCR amplification was made of the pBABE-Bob plasmid using primers described by Deng et al.¹⁴ to which a T7 RNA polymerase site had been added at the 5′ end of the downstream primer. This yielded a product of expected size (562 bp) and sequence. This was used with T7 polymerase in the presence of digoxygenin-labeled UTP (Dig-RNA labeling kit, Boehringer Mannheim/Roche) to make the antisense probe. Probe specificity was confirmed by Southern blot analysis of pBABE-BOB and comparing our Northern blot analysis of a human MTCII panel (Clontech), with results similar to those of Deng et al.¹⁴

In situ hybridization was performed as previously described,²⁶ except that formalin fixed, paraffin-embedded colon tissue was used, and protease digestion was performed with pepsin (2 mg/ml in 0.1 N HCl for one hour at room temperature). After inhibition of endogenous alkaline phosphatase by 20% acetic acid for 15 seconds at 4° C., immunodetection was performed with alkaline phosphatase-conjugated antidigoxygenin and overnight NBT/BCIP staining. Slides treated with RNAse prior to in situ hybridization, but otherwise treated identically, were used as negative controls.

(5) Immunostaining

Except for the microtubule staining mentioned below and the CXCR4 and CCR5 staining (for which air dried, unfixed sections were used), all indirect immunofluorescent staining was done on acetone fixed frozen tissue sections. Cultured cells for microtubule staining were fixed in paraformaldehyde phosphate buffered saline (PBS) as previously described.⁷ The Bob37 and Bob39 antibodies were preincubated with 100-fold excess of the peptides DKEAS . . . or KNYDF . . . respectively, which was found to increase staining specificity, presumably by interfering with nonspecific binding to these epitopes. Thus, the amino terminal sequence MDPEET . . . is presumed to be the best antigen for eliciting antibodies of good specificity in immunostaining. These were incubated in 10% normal goat serum, then the primary antibody at 5 μg/ml for 1 hour, then washed and incubated with Cy-3 conjugated Goat antirabbit or antimouse IgG (1:100 dilution, Jackson Immunoresearch) as appropriate, all at room temperature. Similar preparations omitting the primary antibody were made as negative controls.

Indirect immunofluorescent staining was done on acetone fixed or unfixed frozen sections, using 5 micrograms/ml of the primary antibody at room temperature for 30-60 minutes, then Cy3 conjugated goat antirabbit IgG (Jackson Immunoresearch, West Grove, Pa.) as the secondary antibody.

Initial formalin fixed, paraffin embedded sections demonstrated only very weak staining for Bob. However, good staining with DKEAS . . . -preabsorbed antiBob37 (similar to that of frozen sections) was obtained if the slides were pretreated with pressure cooker triethanolamine-based antigen retrieval using Trilogy (Cell Marque, Austin Tex.) using the manufacturer's procedure.

(6) Cell Culture Studies—Calcium Measurements and Microtubules

HT-29 cells (from ATCC) were grown in DMEM-F12 with 10% bovine calf serum, and were used 24-48 hours after plating. The Ghost (3) cells were obtained from the AIDS Research and Reference Reagent Program, and were grown DMEM with 10% bovine calf serum supplemented with 500 μg/ml G418, 100 μg/ml hygromycin, and 1 μg/ml puromycin.²⁷ These cells were used 24 to 96 hours after plating.

Calcium studies were done on HT-29 cells and Ghost (3) cells loaded with Fluo 4. The cells were placed in Locke's buffer containing 5 μg/ml Fluo 4 AM ester (Molecular Probes) for 20 to 30 minutes, then in Locke's buffer for an additional 30 minutes before testing. Various concentrations of gp120_{IIIB} (X4 trophic), gp120_{CM235} (R5 trophic), gp120_{mN} (X4 trophic), gp120_{93TH975} (nonsyncytium-inducing primary isolate from an asymptomatic subject, trophism undetermined but likely R5), and gp120_{SF2} (X4R5 trophic), ranging from 10 nM to less than 1 pM, were added in Locke's buffer.⁶ The gp120 proteins were obtained from the AIDS Research Reagent Referral Program. Some HT-29 cells were given one of the following pretreatments: 50 μg/ml anti-Bob37 overnight, 1 μg/ml pertussis toxin overnight, or 4.6 μg/ml U73122 for one hour. With the HT-29 cells only, similar tests were also performed with SDF-1, MIP-1α, MIP-1β, and RANTES rather than gp120. The cells were visualized every 4 seconds in a confocal microscope, using 488 nm argon laser excitation and detection filters appropriate for fluorescein-like dyes.

To examine the microtubules, HT-29 cells were treated with 10 nM gp120_{CM235} or 50 pM gp120_{IIIB}, then fixed one hour later in 4 g/100 ml paraformaldehyde PBS, then stained with mouse anti acetylated tubulin (5 μg/ml, Sigma) by a method similar to that given above. Some cells were pretreated with 50 μg/ml antiBob37 overnight, 1 μg/ml pertussis toxin overnight, or 4.6 μg/ml U73122 for one hour before the gp120 was added. Untreated cells were also stained.

(7) Blocking Antibodies

Blocking antibody experiments were done with HT-29 cells (monitoring calcium flux, microtubule loss, or transepithelial resistance), with Ghost (3) Bob cells (monitoring calcium flux) and peripheral blood mononuclear cells (monitoring calcium flux and HIV infection). Specifics of these protocols and the specific results are given herein. Initially a combination of antiBob37 and antiBob39 were used, but in subsequent HT-29 calcium flux experiments it was determined that antiBob37 was the neutralizing antibody and that antiBob39 had no significant effect. Antibody concentrations of 40 to 50 micrograms/ml overnight were needed for maximal effect.

b) Results

Antibody preabsorbtion studies with Ghost (3) cells suggest that antibodies to the N terminal domain epitope (MDPEET . . . ) and the first extra cellular loop epitope (DKEAS . . . ) can induce a greater loss, such as 10 fold, in sensitivity to gp120IIIB-induced calcium flux.

The fact that antiBob37 antibodies are better than the antiBob39 antibodies at neutralizing virus suggests that Bob's N terminal domain, the first extracellular loop, or both contain the activating binding site. Inhibition by pretreatment of the gp120 by heparin suggests that the activating binding on gp120 is in the amino half of the V3 loop.

(1) Western Blots

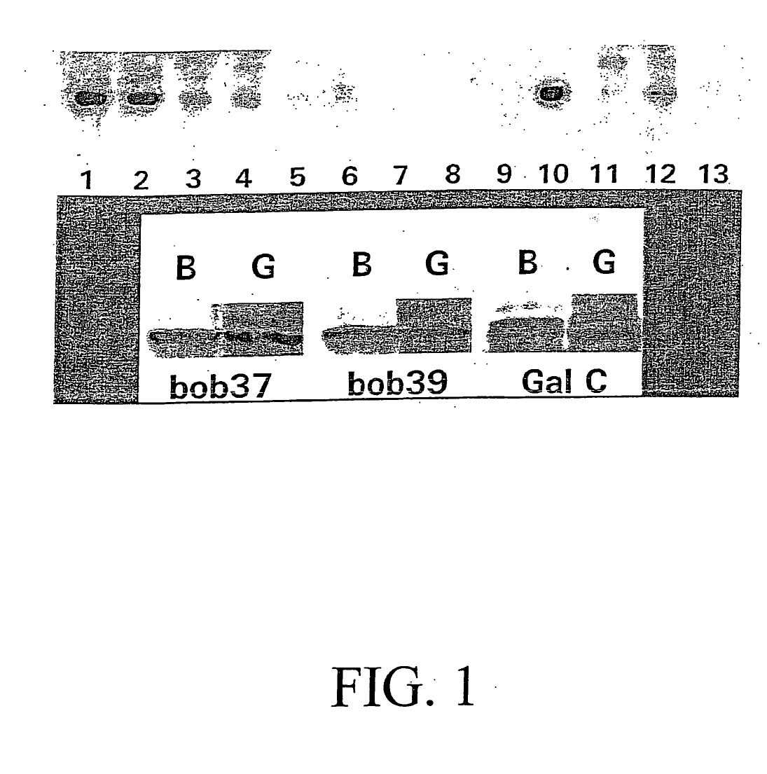

In Western blots, homogenates of small bowel and colonic mucosa, lymph node, prostate, testis, and liver, both antibodies stained bands at 36 kD (FIG. 1a), slightly less than the 40.8 kD molecular weight of Bob estimated from the mRNA sequence. No similar bands were observed in homogenates of brain, placenta, lung, uterus, heart, pancreas, or skeletal muscle. The 36 kD band was absent in Western blots omitting the anti-Bob primary antibody, and the bands were greatly reduced if the anti-Bob was pretreated with excess of the appropriate peptides.

HT-29 homogenates immunoprecipitated with either anti-Bob37 or polyclonal antigalactosyl ceramide were stained similarly with either anti-Bob37, anti-Bob39, or monoclonal antigalactosyl ceramide, resulting in similar 36 kD bands (FIG. 1b). This indicates that both Bob antibodies, formed to different epitopes, recognize the same protein and that both polyclonal and monoclonal antigalactosyl ceramide antibodies also bind this protein.

For the Western blots typically, a range of 0.01 to 0.5 micrograms/ml, each antibody stained a protein band at a relative molecular weight of 36 kD, slightly less than the 40.8 kD anticipated from the Bob mRNA sequence. The 36 kD Western blot bands were virtually eliminated by overnight preincubation with 500-fold excess of the antibodies used as antigens. Control blots without the primary antibody had no 36 kD band at all. These bands were recovered almost exclusively from the cold Triton X-100 soluble fraction, unlike most raft components.

The Western blots were typically done with 50 micrograms protein per lane, in a SDS 10% polyacrylamide gel electrophoresis, then electroblotted onto nitrocellulose. The blots were treated with with 5 g/100 ml nonfat dry milk for one hour in 20 mM Tris buffered saline, pH 7.6, with 0.1% Tween 20. Blots were visualized either by chemiluminescence using ECL reagents (Amersham/Pharmacia, Arlington Heights, Ill.) or by alkaline phosphatase anti rabbit IgG with NBT/BCIP staining.

(2) Bob and Galactosyl Ceramide—Both Cross Reaction and Binding

Prior work had indicated that antibodies to the glycosphyngolipid galactosyl ceramide can induce changes similar to HIV enteropathy in HT-29 cells, or block the similar effect of gp120 (ref Delezay O. Virol 238:231-42, 1997). Also, as demonstrated in the previous example, galactosyl ceramide, like Bob, is present mostly in lipid rafts. Therefore, the relationship between Bob and galactosyl ceramide was studied.

As indicated in FIG. 1, both antiBob37 and monoclonal antiGalactosyl ceramide antibodies are capable of immunoprecipitating Bob, and polyclonal antiGalactosyl ceramide, as well as antiBob37 and antiBob39 are capable of Western blot staining a 36 kD protein corresponding to Bob with very similar bands. This result indicates that antiGalacosyl ceramide antibodies cross react with Bob.

(a) Bob Peptides Bind Galactosyl Ceramide

Artificial membranes composed entirely of galactosyl ceramide with an alpha hydroxylated fatty acid were made. The surface pressure was measured with a microtensimeter (Kibron Inc., Finland) designed for the study of protein-lipid interactions. The peptides used for immunization were added to the subphase, and the surface pressure was continuously measured until equilibrium was reached. The increase in pressure was expressed in mN/m. Significant galacotsyl ceramide binding was seen with peptides corresponding to the three extracellular loops, but not the N terminal domain or the peptides from the cytoplasmic carboxy terminal domain. Results are shown in the table below.

Table 5 shows an interaction of synthetic Bob peptides with GalCer purified from HT-29-D4 rafts. A monolayer of GalCer was prepared at the air-water interface at an initial surface pressure of 10 mN/m. The maximal surface surface pressure increase (Δπmax) reached at the equilibrium is indicated. The values are the mean of three separate experiments (±SD).

TABLE 5

| | | Δπ max |
|---|---|---|
| Peptide sequence | Topology | (mN/m) |
| HAEDFARRRKRSVSL (P1) | Internal C-ter domain | 1.89 ± 0.85 |
| SEQ ID NO: 1 | | |
| DKEASLGLWRTGSFLCK | First extracellular loop | 5.40 ± 1.52 |
| (P2) (SEQ ID NO: 2) | | |
| MDPEETSVYLDYYYATS | Extracellular N-ter end | 2.26 ± 0.12 |
| (P3) (SEQ ID NO: 3) | | |
| SGLRQEHYLPSAILQ (P4) | Third extracellular loop | 5.21 ± 1.06 |
| (SEQ ID NO: 4) | | |
| SRELTLIDDKPYCAEKKAT | Second extracellular | 6.86 ± 1.37 |
| (P5) (SEQ ID NO: 5) | loop | |
| KNYDFGSSTETSDSHLTK | Internal C-ter domain | 2.67 ± 0.40 |
| (P6) (SEQ ID NO: 6) | | |
| P1 + P2 + P3 + P4 + P5 + P6 | — | 8.86 ± 1.73 |
| P1 + P3 + P6 | — | 1.52 ± 0.39 |
| P2 + P4 + P5 | — | 8.61 ± 1.54 |

While certain galactosyl ceramide antibodies can cross react with Bob, immunostaining with Bob37 demonstrates no staining of brain or peripheral nerve, indicating that the antigenic sites recognized by this antibody do not include or cross react with either galactosyl ceramide or myelin basic protein, which are present in abundance in these tissues.

These findings indicate that Bob and galactosyl ceramide can have both immunologic cross reactivity and also that peptides from Bob bind galactosyl ceramide. Myelin basic protein has a similar property, that has been explained by the presence of the modified amino acid citrulline, which is similar to the immunogenic site in galactosyl ceramide (ref McLaurin J, Moscarello M A. J Neurol Sci 108:73-7, 1992). However, the sites recognized by the neutralizing antibody antiBob37 are distinct from galactosyl ceramide and myelin basic protein. An experimental demyelinating disorder (experimental allergic encephalomyelitis) can be induced by immunizing with myelin (ref Genain CP. Immunol Rev 183:159-72, 2001). Antibodies to a related myelin component, MOG, has recently been linked to progressive forms of multiple sclerosis (ref Genain CP et. al: Abstract #WP5 presented at American Neurological Association, October 2002). Thus antibodies interacting with components of myelin sheaths could have serious side effects, but antibodies or other substances binding Bob but not galactosyl ceramide, such as antiBob37, would be more desirable as inhibitors of Bob activation, as related to the myelin issues.

The Bob antibodies discussed herein, however, do not have cross reactivivty with galactosyl ceramide, indicating that the epitopes used for Bob antibody recognition are not epitopes that are recognized by galactosyl ceramide antibodies.

(3) RNA In Situ Hybridization and Immunostaining

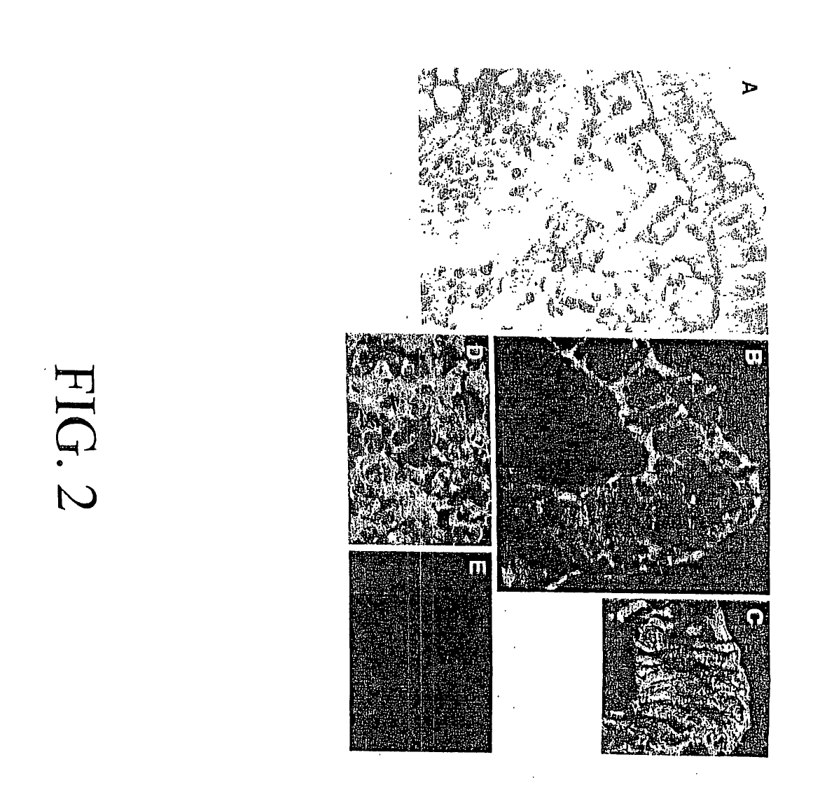

RNA in situ hybridization using a Bob antisense riboprobe showed granular cytoplasmic staining of both surface and crypt epithelial cells and lamina propria mononuclear cells in colonic mucosa (FIG. 2a). Indirect immunofluorescent staining with antiBob antibodies showed extensive staining of intestinal epithelium and lamina propria mononuclear cells. In essentially all small intestinal epithelial cells and in a few colonic crypt cells, there was granular membranous staining, mostly of the basal membrane and just under the brush border of the luminal surface (FIG. 2b). The staining was similar throughout the villi and crypts. In colonic epithelium, Bob staining was mostly granular and cytoplasmic (FIG. 2c), although a few crypt cells had membranous staining resembling that of small bowel.

Either antiBob antibody stained the Bob-transfected Ghost (3) cells in a finely granular, predominantly cytoplasmic pattern, but did not stain the Ghost (3) parent cell line or cells transfected with CCR5 or CXCR4 (FIGS. 2d and e). Both the small bowel and the colonic epithelium had weak staining for CXCR4 and CCR5, which was mostly at or near the luminal surface. Small bowel epithelial cells staining was very weak for both CXCR4 and CCR5.

(4) Calcium Flux Studies

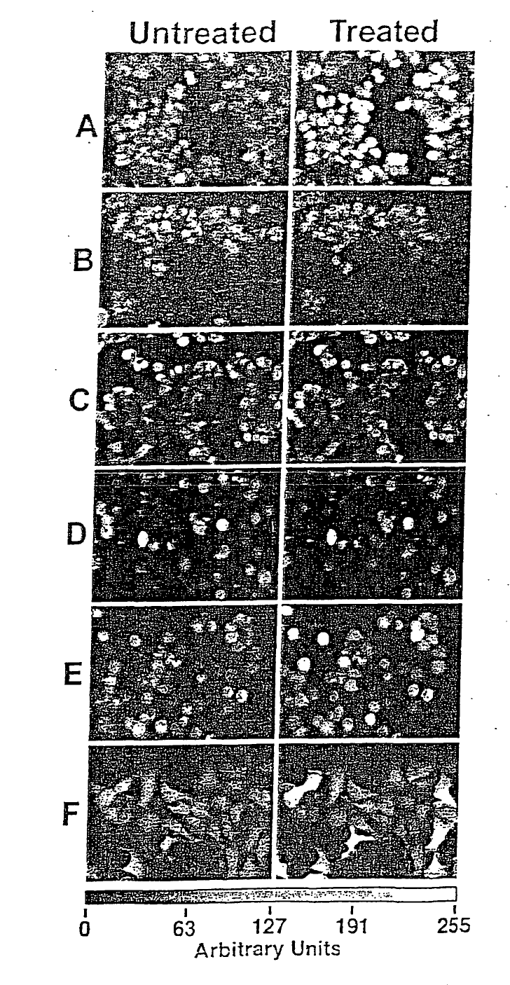

Gp120_{IIIB} and gp120_{CM235} induced an identical, immediate (within 4 seconds) increase in free calcium in HT-29 cells (FIG. 3a). The signals lasted from about 12 seconds to several minutes, and varied from cell to cell. The signals were generally longer when more gp120 was added. Serial dilution studies showed that the minimal concentration causing calcium signaling was 1 pM for gp120_{IIIB} and 2.5 nM for gp120_{CM235}. Gp120_{MN}, gp120_{SF2}, and gp120_{93TH975} induced no calcium signaling, even at 10 nM concentrations.

Each of the natural ligands of CXCR4 and CCR5-100 ng/ml SDF-1, 50 ng/ml MIP-1α or MIP-1β, and 10 ng/ml RANTES—induced similar calcium signaling. Calcium signaling induced by either gp120 or SDF-1 was inhibited by the selective G protein inhibitor pertussis toxin and by the phospholipase inhibitor U73122 (FIG. 3b, c). Pretreatment with antiBob37 antibodies inhibited the calcium signaling by both gp120_{CM235} and gp120_{IIIB} (FIG. 3d). In cells pretreated with antiBob, calcium signaling could then be induced by 100 ng/ml of SDF-1, implying that the CXCR4 receptors and the G protein-induced calcium signaling pathway remained functional (FIG. 3e). Although HIV_{IIIB} is an X4 or T lymphocyte-trophic virus, gp120_{IIB}-induced calcium signaling was not inhibited by antiCXCR4 neutralizing antibodies. Furthermore, the polarized cells could be stimulated by gp120_{IIIB} from either the basal or top surface, and anti-Bob37 neutralized activation from either side (results not shown).

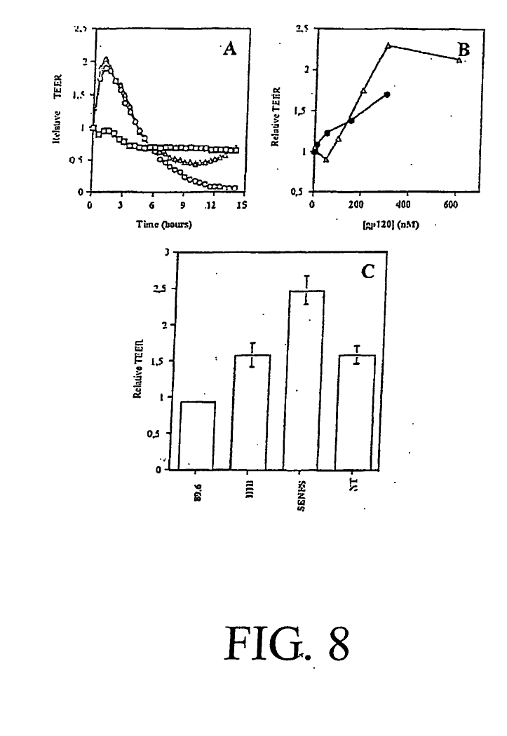

To confirm that Bob is the receptor mediating gp120-induced calcium signals, gp120-induced calcium signals were also examined in Ghost (3) cells transfected to express Bob, CXCR4, or CCR5, as well as the parental cell line.²⁷ Calcium signaling was induced by as little as 0.015 pM gp120_{IIIB} (FIG. 3f) or 8 pM gp120_{CM235} in Bob-transfected Ghost (3) cells, while Ghost (3) CXCR4-transfected cells required at least 150 pM gp120_{IIIB} and did not respond to gp120_{CM235} at any concentration tested. Both Ghost (3) CCR5-transfected cells and the parental cell line (which expresses CXCR4 at low levels) required about 1 nM gp120_{IIIB} and did not respond to gp120_{CM235} at any concentration tested. Gp120 from each of the three viral strains (MN, SF2, and 93TH975) which did not cause calcium signals in HT-29 cells similarly did not cause calcium signals in the Ghost (3) Bob-transfected cells at any concentration tested.

The calcium signals in the Ghost (3) cells were usually very intense and often lasted for several minutes. At the minimal concentrations, only some of the cells exhibited calcium signals (FIG. 3f), but at higher concentrations most cells exhibited signaling. However, at very high gp120 concentrations, calcium signaling became weak or negative.

(5) Microtubule Staining

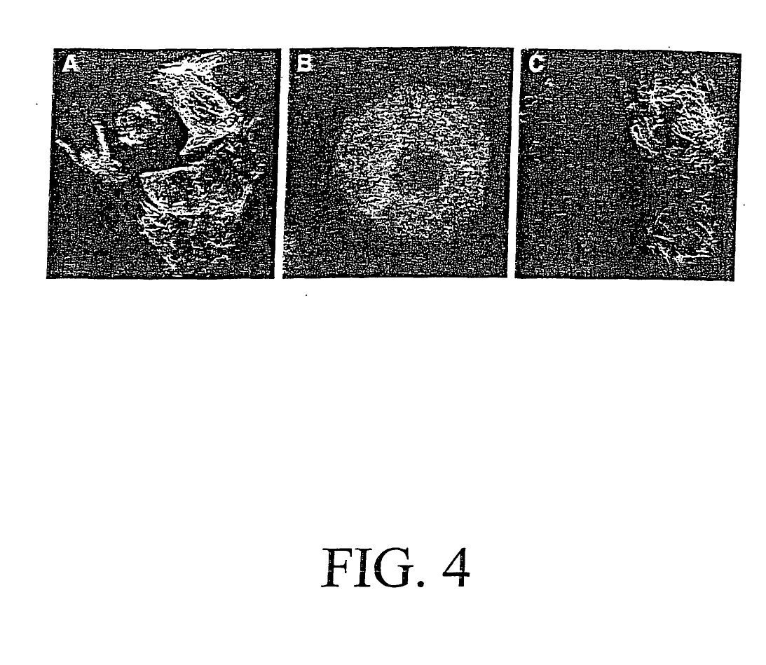

It was demonstrated that gp120-induced microtubule alterations in HT-29 cells. Either gp120_{IIIB} or gp120_{CM235} caused a marked loss of acetylated tubulin-staining microtubules (FIG. 4a, b). This effect was most apparent in clusters of several cells—large cell clusters (>20 cells) often retained stainable microtubules. The loss of microtubule staining was inhibited by pretreatment with antiBob (FIG. 4c), pertussis toxin, or U73122 under the same conditions used for calcium signaling, except that the cells were in DMEM-F12 rather than Locke's buffer. The loss of microtubules induced by gp120_{IIIB}, and that this effect could be inhibited by antiBob, was confirmed with differentiated, polarized HT-29 cells.

Both in situ hybridization and immunostaining with two different antibodies demonstrate that Bob is present in intestinal epithelium and in lymphocytes. Also, Bob is abundantly expressed at the basal and apical surfaces of small bowel epithelium. In agreement with prior results¹² and in contrast to Bob, CCR5 and CXCR4, while present in intestinal epithelium, are mostly at the luminal surface. In chronic HIV infection, most productively HIV-infected cells in intestinal mucosa are superficial lamina propria macrophages;¹³ intestinal epithelium is probably exposed to gp120 on the basal surface. Thus Bob, unlike CCR5 or CXCR4, is present at the correct site to mediate gp120-induced signaling.

The inhibition of the gp120-induced calcium fluxes and microtubule loss in HT-29 cells by pertussis toxin and U73122, as well as induction of similar calcium signals by the natural ligands of CCR5 and CXCR4, show a G protein coupled receptor/pertussis toxin-sensitive G protein/phospholipase mechanism. Both strains of gp120 that induce these changes are inhibited by affinity purified anti-Bob antibodies. Also, picomolar or lower concentrations of gp120 of either strain induced calcium fluxes in Bob-transfected Ghost (3) cells but not in the parental cell line or in CCR5 or CXCR4-transfected Ghost (3) cells. All three gp120's of strains not inducing signaling in HT-29 cells also do not induce calcium fluxes in Bob-transfected Ghost (3) cells. Thus, this indicates Bob mediates these gp120-induced effects in HT-29 cells, which are very similar to those of HIV enteropathy.⁷

Thus, Bob is present at the right site, Bob induces physiologic changes in HT-29 cells resembling HIV enteropathy, Bob cross reacts with anti-galactosyl ceramide antibodies, and that, unlike the principal coreceptors CCR5 and CXCR4, Bob induces calcium signaling at extremely low gp120 concentrations. A decrease in acetylated tubulin staining in the intestinal epithelium of HIV-infected subjects has also been shown which indicates decreased microtubule stability in vivo.⁸ Thus, gp120-induced Bob activation causes calcium signaling and microtubule loss which is associated with in HIV-associated enteropathy and other HIV-related effects.

One cell signaling pathway induced gp120 caused HIV enteropathy is the following. Gp120 activates intestinal epithelial cell Bob, inducing G protein and phospholipase activation and thus inositol triphosphate and calcium signaling. Increased cytosolic calcium depolymerizes microtubules.²⁹ Microtubule loss alters RhoA and RacI activation, increasing actin-myosin contraction³⁰ near the tight junctions, causing increased paracellular permeability and diarrhea.³¹ Microtubule loss also reduces enterocyte lipid transport,⁹ causing lipid malabsorption.

Prior studies of coreceptor activation by gp120 focused almost exclusively on the major coreceptors CCR5 and CXCR4, with the implicit assumption that the activation was an incidental phenomenon with receptors for which viral fusion was the main goal. Finding extremely sensitive gp120-induced activation of Bob, a very inefficient coreceptor, by both an X4 trophic strain (HIV_{IIIB}) and an R5 trophic strain (HIV_{CM235}) indicates that different receptors can be activated than those used as maincoreceptors for viral fusion.

(6) Tissue and Cellular Localization of Bob

Tissue homogenates were made from a variety of normal human tissues obtained as excess tissue from surgical pathology specimens or from autopsies performed within one day of death. The tissue was frozen at less than −70° C., then thawed and homogenized at 0-4° C. in Tris buffered saline, pH 7.6 with 1% Triton X-100 and a combination of protease inhibitors (Complete (TM) protease inhibitor tablets, Boerhinger Mannheim). Tissue culture cells (HT-29 cells and Ghost (3) cells) were lysed and handled in a similar fashion. Supernates were assayed for protein content (Pierce BCA assay). The homogenates were tested in Western blots as discussed herein. Results are as follows: Abundant Bob was detected in colonic mucosa, small bowel mucosa, lymph node, testis, prostate, and liver (FIG. 1). No Bob was detected in brain, placenta, lung, uterus, heart, pancreas, or smooth or skeletal muscle. A 36 kD Western blot band corresponding to Bob was also found in kidney, and in HT-29 cells and Bob-transfected Ghost (3) cells, but not the Ghost (3) parent cell line. Additional studies demonstrated Bob in renal tissue in Western blots. Immunostaining demonstrated Bob in renal tubular cells and, to a lesser degree, in glomerular epithelium.

Indirect immunofluorescent staining for Bob was found in two patterns, either granular cell membrane staining (most lymphocytes, small bowel, and germinal epithelium of the testis) or granular cytoplasmic staining, usually with some additional cell membrane staining (colonic and rectal epithelium, prostate, HT-29 and Ghost (3) Bob cells). The pattern of staining in intestinal epithelium included both apical and basolateral cell membrane staining, unlike the mainly apical staining for CCR5 and CXCR4. The liver had a unique pattern of bile canalicular/pericanalicular granular staining. Coimmunostaining with FITC-labeled Cholera toxin B subunit (a marker for GM1 and thus of lipid rafts/glycosphyngolipid-enriched microdomains, staining done at 0-4° C.) demonstrated that the granular cell membrane staining for Bob colocalized with the Cholera toxin, indicating that the granular cell membrane staining corresponded to lipid rafts.

RNA in situ hybridization, done with a digoxygenin-labeled riboprobe prepared by PCR from the pBABE-Bob plasmid (courtesy of Dr. Dan Littman and the AIDS Research and Reference Reagent Program with a T7 promotor site. RNA was then synthesized using T7 polymerase in the presence of digoxygenin-labeled UTP. In situ hybridization was done on formalin fixed, paraffin embedded normal rectal tissue sections after the following pretreatments: pepsin digestion (2 mg/ml in 0.1 N HCl for one hour at room temperature, then inhibition of endogenous alkaline phosphatase by 20 acetic acid for 15 seconds at 4° C. Slides treated with RNAse before the in situ hybridization were used as negative controls. The sections showed good staining of rectal crypt and surface epithelium, as well as essentially all the lamina propria lymphocytes.

(7) Bob Activation in Other Cells

Bob is abundantly present in lymphoid tissue by Western blot, and it is present in most lymphocytes, based on both mRNA in situ hybridization and immunostaining evidence. In lymphocytes, it is in a peripheral cell membrane granular pattern, often limited to one side of the cell.

HIV induces derangements of inositol polyphosphate metabolism and calcium flux in lymphocytes, and that these changes are promoted by HIV envelope surface protein gp120 (Kornfeld H Nature 335:445-8, 1988). These changes likely both promote HIV infection and subtly alter immune function. This was partially explained when it was discovered that the HIV envelope surface protein, gp120, induces activation of the major coreceptors CCR5 and CXCR4 (Arthos J. J Virol 74:6418-24, 2000, Herbein G. Nature 395:189-94, 1998, and Weissman D. Nature 389:981-5, 1997), but this cannot address many issues with HIV activation—these receptors were activated only at relatively high gp120 doses (200 picomolar to nanomolar) while in vivo concentrations, at least in blood, were generally subpicomolar.

Human osteosarcoma cell lines were transfected to express CD4, one or another of the HIV coreceptors, and a Green Fluorescent Protein reporter for active HIV infection, for the purpose of testing which of the known coreceptors was used to induce HIV infection by a given HIV strain (Morner A. J Virol 73:2343-9, 1999 and Deng K Nature 388:296-300, 1997). These cells were obtained from the AIDS Research and Reference Reagent Program, courtesy of Dr. Vineet KewalRamani and Dr. Dan Littman. These cells were grown in DMEM with 10% bovine calf serum supplemented with 0.5 mg/ml G418, 0.1 mg/ml hygromycin, and 1 microgram/ml puromycin, and were used at low passage number (less than 10). We have discovered that these cell lines are also activated by gp120 of certain viral strains, inducing a calcium flux in these cells.

Within 4 days of plating, coreceptor-transfected Ghost (3) cells (as well as the parent cell line not transfected to express any HIV coreceptors as a control) were put in Locke's medium, then treated with Fluo-4 (5 micrograms/ml for 30 minutes at 37° C.). After an additional 30 minute incubation in Locke's buffer at room temperature, the cells were examined in a confocal microscope while gp120 of various viral strains was added, and the cells were examined at 4 second intervals to look for a gp120-induced calcium flux. The results are given in the following table, expressed as the minimal concentration of gp120 resulting in a significant calcium flux. “−” means that no reproducible calcium flux was observed. “ND” means that the gp120 of that viral strain was not tested with the stated coreceptor. Various concentrations of gp120 from 250 nM to 1 pM was done for each of the cell lines, and further dilutions were done if calcium fluxes were observed at 1 pM.

gp120_{IIIB} is an extremely potent activator for Bob activation. See Table 3.

TABLE 3

| | HIV strain | | | | |
|---|---|---|---|---|---|
| Coreceptor | IIIB | CM235 | SF2 | 93TH435 | MN |
| Fusion | X4 | R5 | X4R5 | probable | X4 |
| trophism | | | | R5 | |
| Bob | 15 fM | 10 pM | — | — | — |
| Parental | 250 nM | — | — | — | — |
| CXCR4 | 150 pM | — | — | — | — |
| CCR5 | 250 nM | — | — | 100 nM | — |
| CCR1 | — | — | — | — | ND |
| CCR2b | 250 nM | — | — | — | ND |
| CCR3 | — | — | — | — | ND |
| CCR4 | — | — | — | — | ND |
| CCR8 | — | — | — | — | ND |
| CX₃CR1 | — | — | — | — | ND |
| CXCR6/Bonzo | — | — | — | — | ND |

Abbreviations:
nM = nanomolar,
pM = picomolar,
fM = femtomolar,
— = no calcium flux seen at any dilution tested.

Table 3 shows Ghost (3) cells, transfected to express one of the known HIV coreceptors as stated on the left column, were loaded with Fluo-4 to determine the cytosol calcium content. These cells were treated with serial dilutions (100 or 250 nM to at least 1 pM, or even less if calcium fluxes were noted) of gp120. Gp120s from each of four HIV-1 strains: IIIB, CM235, SF2, and 93TH435 were used. Listed is the greatest dilution which induced a significant, reproducible calcium flux. The parental line is known to express CXCR4 at very low levels. Those results indicated by an asterisk are thought to represent this artifact.

There was activation of Bob, CXCR4, and CCR5 in a viral strain-specific manner. While CCR2b activation was seen with the IIIB strain, this was similar to that of the parental cell line. The parental cell line was not transfected to express a coreceptor, but contained small quantities of CXCR4. Activation of gp120 by Bob occurred at a wide range of gp120 concentrations, down to 15 fM for the IIIB strain and 10 pM for the CM235 strains. Activation of CXCR4 requires at least 150 pM, and CCR5 activation requires 100 nM. Thus, Bob was 10,000 fold more sensitive to gp120-induced activation than the next most sensitive receptor, CXCR4. HIV CM235 activates Bob and, at 100 fold greater gp120 concentrations, than CCR2B. HIV 93TH435 activates CCR5, but only at high gp120 concentrations.

Bob is the only receptor activated by less than 150 pM of any gp120. There is 1 pM of gp120 or less in the blood of HIV-infected subjects, although it is likely more abundant in some tissues. Thus Bob is the only coreceptor that is activated at clinically relevant gp120 concentrations in blood or in tissues with low abundance of gp120.

It is consistent that gp120-induced Bob activation induces similar calcium signaling in other Bob-expressing cells such as lymphocytes, explaining the long known gp120-induced alterations in lymphocyte inositol phosphate metabolism.³³ It is also consistent that Bob activation could inhibit the anti-HIV immune response or induce the gp120_{IIIB}-induced, actin-mediated colocalization of CD4 and the major coreceptors advantageous for HIV infection.³⁴ This is shown herein.

Normal human peripheral blood mononuclear cells were isolated by Ficoll gradient centrifugation, and loaded with the calcium sensitive dye Fluo-4. HIV gp120_{IIIB} induced strong calcium fluxes in the range of 7 nM to 50 pM, and there was a weaker signal at 5 pM. Preincubation with antiBob37, 10 micrograms/ml for 12 hours, eliminated the calcium flux induced by gp120IIIB, even at 7 nM.

(8) Gp120—Induced Peripheral Blood Mononuclear Cell Activation

Experiments corresponding to those with HT-29 cells were done with peripheral blood mononuclear cells. These were isolated from the blood of a healthy, HIV-uninfected male by a Ficoll-Hypaque techique (Histopaque, Sigma), then loaded with Fluo-4 by a technique nearly identical to that used for HT-29 cells, except that Hank's buffer was used rather than Locke's, and the cells were centrifuged (2,000 g for 10 minutes) just before calcium flux measurements to improve adherence. Gp120-induced calcium fluxes were documented with as little as 5 pM gp120_{IIIB}. Overnight incubation incubation in RPMI1640 plus 10% fetal calf serum reduced the sensitivity to gp¹²⁰_{IIIB} to 500 pM to 5 nM. However, after an overnight incubation with 50 micrograms/ml of antiBob37, no calcium fluxes were detected, even with 5 nM or 50 nM added gp120_{IIIB} (at least 10-fold inhibition). Similarly, a 15 minute preincubation of the gp120_{IIIB} with 40 micrograms/ml sodium heparin at 0-4° C. inhibited the calcium flux, even at 200 nM gp120_{IIIB} (40-fold inhibition).

This blocking antibody effect, along with the low picomolar concentrations of gp120IIIB used (which induce only Bob among the coreceptors tested disclosed herein), indicate that calcium fluxes in normal human lymphocytes are induced by gp120 via Bob. It has been shown that phospholipase C and inositol triphosphate are involved in this signaling mechanism, thus Bob activation is a cause consistent with the deranged inositol polyphosphate metabolism seen in AIDS. Gp 120 induced actin-based contraction of the IIIB strain renders cells more susceptible to HIV-1 infection. (Iyengar S, Hildreth J E K, Schwartz D H. Actin-Dependent Receptor Colocalization Required for HIV Entry into Host Cells. J Virol 72:5251-55, 1998) Calcium signaling is widely known to induce actin-based contraction. Herein it is shown that calcium signaling is caused by gp120/Bob interactions at very low concentrations of gp120. Thus Bob activation is consistent as being the cause of HIV infection of CD4 lymphocytes.

(9) GP120—Induced Intestinal Cell Line Activation

Experiments with both undifferentiated and differentiated forms of the intestinal HT-29 cell line have demonstrated changes when treated with HIV gp120. For the first two or three days after plating, gp120 induces an immediate (within 4 seconds) calcium flux in undifferentiated HT-29 cells. The cells had previously been loaded with Fluo-4 AM ester (Molecular Probes, Eugene Oreg.) 5 micrograms/ml in the presence of Pleuronic F-127 0.02% in Locke's buffer for 30 minutes, then incubated at room temperature for an additional 30 minutes. Gp120_{IIIB} was added while the cells were on an inverted confocal microscope using a 488 nm argon laser, with detection filters suitable for fluorescein-like dyes. A ten-fold dilution series was done, and the lowest dose of gp120_{IIIB} producing a calcium flux was 1 pM, in good agreement with a prior study. (ref Dayanithi G. Cell Calcium 18:9-18, 1995) This effect was inhibited by at least ten fold (no response at 10 pM gp120) by an overnight prior incubation with antiBob37, 50 micrograms/ml. Similar inhibition was noted with overnight incubation with pertussis toxin (Sigma, 1 microgram/ml) or with a one hour incubation with U73122 (Sigma, 4.6 microgram/ml) but not an equivalent dose of a related, inactive compound U73343. Similar gp120-induced calcium fluxes were noted with gp120 from strain CM235 (down to 2.5 nM), but not strains SF2, MN, and 93TH975. No inhibition was seen with antiBob39, 50 micrograms/ml overnight.

Similarly treated undifferentiated HT-29 cells (except that Fluo-4 was not added) were fixed in paraformaldehyde phosphate buffered saline one hour after gp120 treatment. A marked loss of microtubules was noted in the gp120_{IIIB} treated cells, but not in untreated controls or in cells pretreated with antiBob37, pertussis toxin, or U73122.

Qualitatively similar changes in calcium flux and in microtubules were seen in differentiated, polarized HT-29-D4 cells using methods and reagents as discussed herein (data not shown). Also, gp120 induced substantial changes in transepithelial resistance (a greater than 80% decrease in resistance after 16 hours, following a transient increase in resistance). Pretreatment with antiBob reduced the changes in resistance by approximately 70%.

### 2. Example 2

a) Materials and Methods

(1) Materials

Six peptides corresponding to the deduced amino acid sequence of human GPR15/Bob (Deng et al., 1997) were synthesized and purified by high performance liquid chromatography: HAEDFARRRKRSVSL (P1) (SEQ ID NO:1), DKEASLGLWRTGSFLCK (P2) (SEQ ID NO:2), MDPEETSVYLDYYYATS (P3) (SEQ ID NO:3), SGLRQEHYLPSAILQ (P4 (SEQ ID NO:4)), RELTLIDDKPYCAEKKAT (P5) (SEQ ID NO:5), and KNYDFGSSTETSDSHLTK (P6) (SEQ ID NO:6). Rabbits were injected with either the first three (P1-P3) or second three (P4-P6) peptides, and immune sera were affinity purified with the corresponding antigen peptides. The antibodies were named Bob37 and Bob39 respectively. The anti-CXCR4 mouse monoclonal antibody (clone 12G5) was purchased from R&D. Rabbit anti-GalCer antibodies were from Chemicon. The anti-α-tubulin antibody (clone B-5-1-2) and rhodamine (TRITC)-labeled phalloidin were from Sigma. The soluble analog of GalCer CA52 (Fantini et al., 1997).

(2) Cell Culture

HT-29-D4 cells were routinely grown in 75-cm² flasks (Costar) in DMEM/F12 medium (Biowhittaker) supplemented with 10% fetal calf serum (Dutscher). To induce differentiation, half-confluent HT-29-D4 cells were grown in glucose-free DMEM (Sigma) supplemented with 5 mM galactose and 10% dialyzed fetal calf serum, as previously reported (Fantini et al., 1989).

(3) Virus Production

HIV-1 viruses were produced in peripheral blood mononuclear cells (PBMC). The isolates used in this study were the laboratory strain HIV-1(IIIB) (Popovic et al., 1984) and two primary isolates, HIV-1(89.6) (Collman et al., 1992) and HIV-1(SEN), obtained from a lymph node biopsy (Hammache et al., 1998). Surface envelope glycoproteins were purified by lectin affinity chromatography as previously reported (Hammache et al., 1998).

(4) Immunocytochemistry

HT-29-D4 cells grown on glass coverslips were fixed with 4% paraformaldehyde (w/v) in 0.1M phosphate buffer, pH 7.4, for 30 min at room temperature. The cells were then treated with 50 mM NaCl (15 min), rinsed with phosphate buffer containing 1% bovine serum albumin (BSA), and permeabilized with 0.2% Triton X-100 in phosphate buffer containing 5% BSA (30 min). For α-tubulin staining, coverslips were incubated in a humid atmosphere at 4° C. for 4 hr with the primary antibody (5 μg/ml) in phosphate buffer containing 1% BSA. After washing, the cells were incubated with fluoresecin-conjugated secondary antibodies for 90 min at 4° C. The coverslips were mounted in a Mowiol solution and analyzed by confocal microscopy (Leica TCS inverted laser scanning microscope) as previously reported (Delézay et al., 1997b). For actin staining, rhodamine-phalloidin was added to the secondary antibody solution. To determine the saturating concentrations of anti-GPR15/Bob, anti-CXCR4, and anti-GalCer antibodies, HT-29-D4 cells were incubated with various dilutions of each antibody and revealed with appropriate peroxidase-coupled secondary antibodies (Delezay et al., 1997b).

(5) Transmission Electron Microscopy

The cells were fixed in situ with 2.5% glutaraldehyde in 0.1M phosphate buffer (pH 7.3) for 1 hr at room temperature, washed for 10 min in the same buffer with 6.84% saccharose and post-fixed for 1 hr in 2% osmium tetroxide. After dehydration in graded ethanol, the cells were embedded in epon. Sections were cut perpendicularly to the monolayer and observed with a CM 10 Philips electron microscope.

(6) Electrophysiological Measurements

HT-29-D4 cells were cultured in two-compartment cell culture chambers on a polycarbonate filter (Transwell-clear, Catalog No. 3450, Costar) and analyzed for electrical parameters in modified custom made Ussing chambers as previously reported (Fantini et al., 1989). The chamber was maintained at 37° C. on a hot plate. Apical and basal compartments were filled with electrophysiological medium: 137 mM NaCl, 5.36 mM KCl, 0.4 mM Na₂HPO₄, 0.8 mM MgCl₂, 1.8 mM CaCl₂, 20 mM N-2-hydroxyethylpiperazine-N′-2-ethanesulfonic acid (HEPES), pH 7.4. Transepithelial potential difference was measured with Ag electrodes and continuously recorded using a voltage clamp unit (Physiologic Instrument). Bipolar current pulses (20 μA for 2 s, every 30 s) were passed through Ag electrodes to measure the transepithelial resistance (TEER), which was determined according to the Ohm's law (Delézay et al., 1997b). The sodium-dependent electrogenic glucose transport activity was measured as an increase of short-circuit current (ΔIsc) following apical incubation of HT-29-D4 cells with the nonmetabolizable analog of glucose α-methylglucose (Fantini et al., 2000a). Except when indicated, purified gp120s were incubated in the apical compartment of the cell culture chamber (total volume 1.5 ml instead of 10 ml for the basal compartment of the Ussing chamber device unit).

(7) Surface Pressure Measurements

Galactosylceramide with an α-hydroxylated fatty acid (referred to as GalCer throughout this study) was either prepared from bovine brain (Sigma) or purified from HT-29-D4 membrane rafts (Fantini et al., 2000a). The surface pressure was measured with a microtensiometer (Kibron Inc., Finland) specially designed for studying protein-lipid interactions (Fantini et al., 2000a). The experiments were carried at 25° C. out in a laboratory equipped with a filtered air supply. A solution of GalCer was prepared extemporaneously in hexane:chloroform:methanol, 11:5:4 (v:v:v) at a concentration of 1 mg/ml. The lipid was carefully spread onto the meniscus of a pure water subphase (800 μl). The proteic ligand (GPR15/Bob peptides or HIV-1 gp120) was injected into the subphase with a 10 μl Hamilton syringe. The surface pressure was continuously measured as a function of time until reaching equilibrium corresponding to the maximal surface presure increase (Δπmax) expressed in mN/m.

b) Results

(1) Effect of HIV-1 gp120 on Cytoskeletal Organization in a Model Intestinal Epithelium

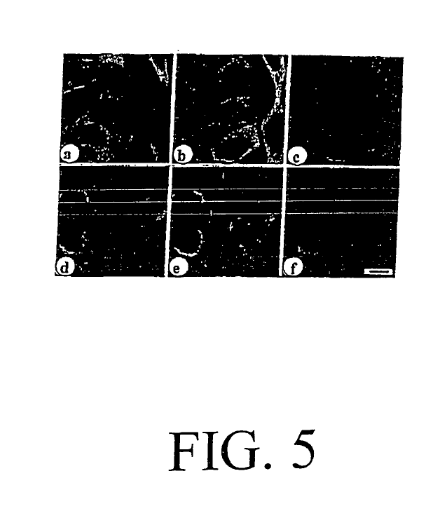

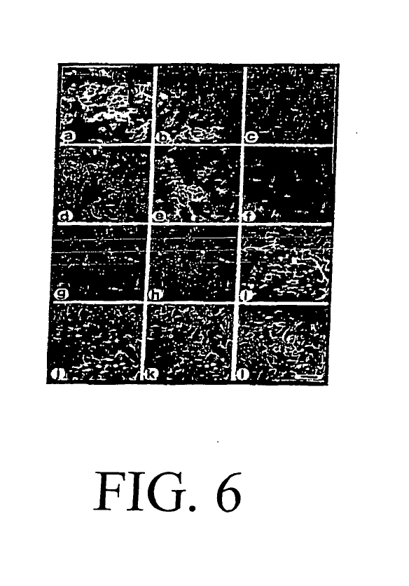

The staining of α-tubulin and actin in HT-29-D4 cells is shown in FIG. 5 (a-c). The α-tubulin labeling was particularly intense in the medium part of the cells where it formed a complex network of microtubules surrounding the nucleus (a, b). Actin filaments were observed in most confocal planes, especially in the basal part of the cells (c). After 1 hr of incubation with gp120 purified from HIV-1 (IIIB) virions, a dramatic decrease in the staining of α-tubulin was observed in most cells, whereas the pattern of actin labeling was poorly affected (FIG. 5, d-f). At lower magnification, numerous lumina or cysts heavily stained with rhodamine-phalloidin were found in gp120-treated cells, but not in control cells. Therefore, the presence of such cysts in HT-29-D4 cell monolayer was carefully analyzed by single staining with rhodamine-plalloidin. Exposure of differentiated HT-29-D4 cells to either HIV-1(IIIB) virions for 16 hr (FIG. 6, e-h), or to purified gp120 (IIIB) for 1 hr (FIG. 6, i-1), resulted in the appearance of cysts in about 5% of cells. In contrast, cysts were usually not present in control HT-29-D4 cells (FIG. 6, a-d). Confocal microscopy analysis provided some insights into the tridimensional organization of these cysts. Indeed, according to the confocal plane, the staining of these structures evolved from a rounded-shape patch to a bright fluorescent ring. Based on these observations, it was conclude that these cysts correspond to vesicles lined with a membrane closely associated to actin.

(2) Effect of HIV-1 on the Ultrastructure of Differentiated Intestinal Cells

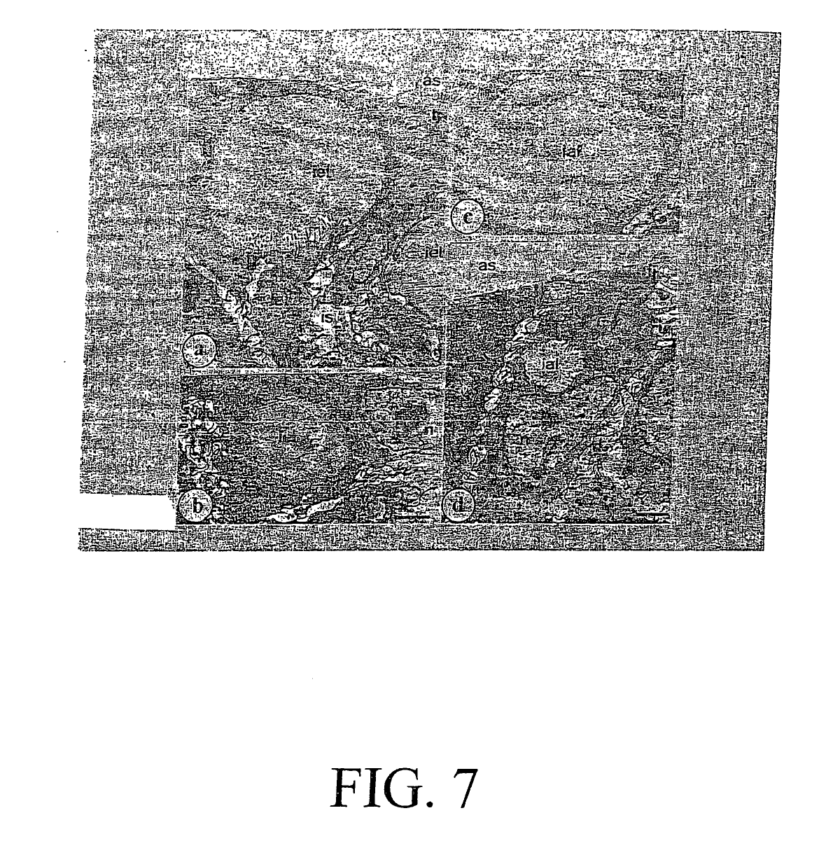

Next, the ultrastructural changes induced by HIV-1 in differentiated HT-29-D4 cells was analyzed. In these experiments, the cells were exposed to either HIV-1(IIIB) virions for 16 hr (FIG. 7, a-c), or to purified gp120 (IIIB) for 1 hr (FIG. 7, d). The main ultrastructural change during incubation with virions or gp120 was the presence of intra- and inter-cellular lumina that could be detected in about 5% of the cells. The lumina were characterized by the presence of either well-organized (a-c) or abortive (d) microvilli. These results were in perfect agreement with the confocal microscopy study. Moreover, the apical plasma membrane of cyst-containing cells was generally devoid of brush border microvilli (a, d). None of these ultrastructural features were observed in the absence of HIV-1. In particular, the apical plasma membrane of control HT-29-D4 cells displayed a regular arrangement of microvilli (e).

(3) Effect of Purified HIV-1 gp120 on Intestinal Barrier and Absorption Functions

Differenfiated HT-29-D4 cells cultured on permeable filters were incubated with purified gp120 (IIIB isolate) in complete, serum-containing medium. After 16 hr of incubation, the transepithelial resistance (TEER) was decreased by 81% (Table 4).

TABLE 4
HIV-1 gp120-induced impairement of intestinal barrier and
absorption functions.

| | | ΔIsc after |
|---|---|---|
| | | α-methylglucose |
| | | treatment |
| Treatment | TEER (Q · cm²) | (μA · cm⁻²) |
| Control (16 hr) | 1,569 ± 225 | 1.18 ± 0.13 |
| +gp120 (16 hr) | 300 ± 108 | 0.35 ± 0.07 |
| Control (48 hr) | 1,228 ± 384 | 1.44 ± 0.25 |
| +gp120 (48 hr) | 320 ± 267 | 0.29 ± 0.11 |

Differentiated HT-29-D4 cells cultured in Transwell chambers were either not treated (control) or treated with 50 nM of purified gp120 (IIIB isolate) for the time indicated in serum-containing culture medium. Transepithelial resistance (TEER) values and short-circuit current variations (ΔIsc) after incubation with 3 mM α-methylglucose (sodium-dependent glucose transport activity) were measured as described in Materials and Methods. The results are expressed as the mean SD of 4 separate experiments.

At the same time, the electrogenic activity of the apical sodium/glucose cotransporter (SGLT1) was reduced by 70%. After 48 hr of treatment with the purified gp120, TEER and SGLT1 activity were decreased by 74 and 80%, respectively. The electrophysiological activity of HT-29-D4 cells was continuously recorded over a period of 14 hr to show the kinetics of gp120-induced defects in barrier and absorption functions (FIG. 8A). The cells could not be bathed in complete medium which interfered with real-time electrode recordings. Instead, a buffer specifically designed for electrophysiological studies was used (Delezay et al., 1997b). Under these conditions, the TEER of control cells was slightly decreased after 4 hr of analysis, but it remained stable for the next 10 hr. When the cells were incubated with gp120 (IIIB isolate), the TEER was dramatically increased during the first hour, then it gradually decreased to null values after 12 hr. Similar variations of TEER were observed during the incubation with gp120 purified from a primary isolate, HIV-1(SEN), that does not infect HT-29 cells (Hammache et al., 1998). In this case however, TEER was found to increase again after 10 hr of incubation with the gp120, so that it recovered a value identical to control cells after 14 hr (FIG. 8A). The initial increase in TEER induced by gp120(IIIB) and gp120(SEN) was a saturable, dose-dependent process (FIG. 8B). 20 min after the addition of gp120, the TEER was enhanced by 2.52±0.19 and 1.60±0.17 (n=3) for gp120(SEN) and gp120(IIIB), respectively. The effect was strain-specific since gp120 from 89.6, another primary isolate that does not infect HT-29 cells (Hammache et al., 1988), was totally inactive (FIG. 8C). A similar increase of TEER was measured upon treatment of the cells with neurotensin, a potent activator of inositol triphosphate-dependent calcium signaling in intestinal cells (Dayanithi et al., 1995).

(4) Lack of Effect of Purified HIV-1 gp120 on Chloride Secretion

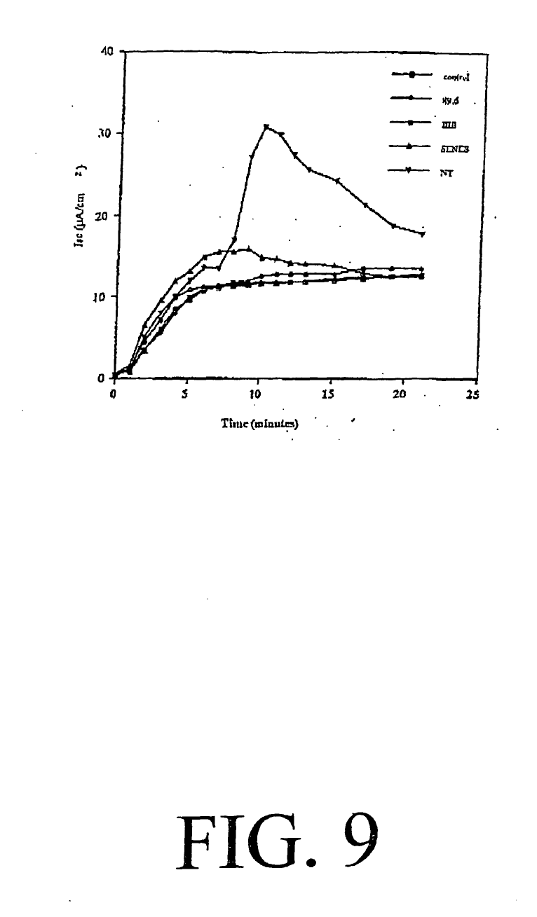

It is well established that abnormal stimulation of intestinal chloride secretion through the cAMP regulated channel CFTR induces watery diarrhea (Kunzelmann, 1999). gp120 coul was shown herein to stimulate cAMP-dependent chloride secretion. As shown in FIG. 9, the cAMP activator forskolin induced a dramatic increase of the short-circuit current (Isc), corresponding to a net apical chloride secretion through CFTR. None of the gp120s tested did induce such an effect in HT-29-D4 cells. The cAMP-dependent chloride secretion could be potentiated by calcium agonists. Indeed, addition of neurotensin after forskolin induced an additive variation of Isc (FIG. 9). Since gp120 virotoxins activate calcium signaling pathways in HT-29-D4 cells (Dayanithi et al., 1995), it was postulated that HIV-associated diarrhea could, at least in part, result from calcium-dependent potentiation of chloride secretion (Fantini et al., 2000a). The effect of gp120 after forskolin activation of CFTR showed no additive effect of Isc (FIG. 9). Taken together, these data show that gp120 virotoxins do not activate nor potentiate chloride secretion in resting or stimulated HT-29-D4 cells.

(5) Protection of HT-29-D4 cells from HT-29 gp120-Induced Enteropathy

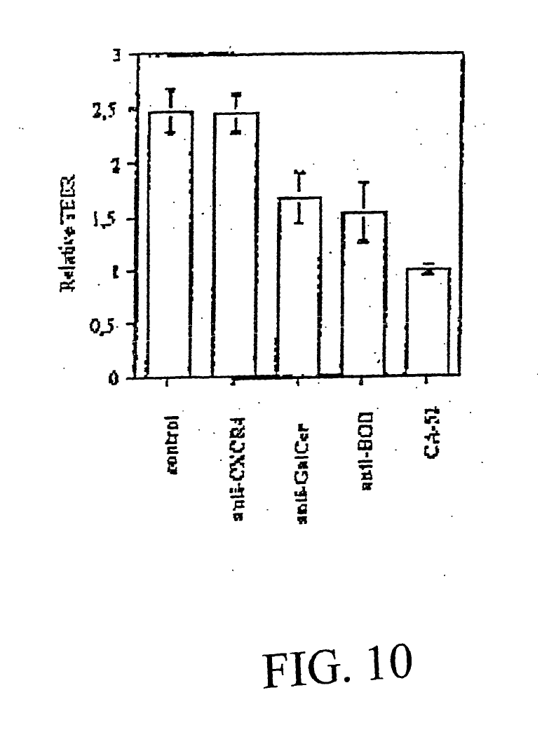

Overall, the most dramatic electrophysiological effect of gp120 on differentiated HT-29-D4 cells is a gradual decrease of TEER systematically preceded by a transient increase which is characteristic of activators of the calcium pathway. This early TEER response is almost complete after 20 min of incubation with gp120(IIIB) or gp120(SEN), and is not observed with gp120(89.6). It is therefore indicative of the enterotoxicity of a gp120. Electrophysiological signature of gp120 virotoxins were used to develop a reliable assay for screening potential protective agents of a gp120-induced experimental enteropathy. In this assay, HT-29-D4 cells were incubated with a saturating concentration of the most potent gp120, i.e. 300 nM of gp120(SEN), in competition with the tested agent. As shown in FIG. 10, neutralizing anti-CXCR4 antibodies did not affect the gp120-induced TEER response. In contrast, a significant protection was achieved by anti-GR15/Bob antibodies, anti-GalCer antibodies, and a synthetic soluble analog of GalCer.

(6) Involvement of GalCer Rafts in the Pathogenesis of HIV Enteropathy

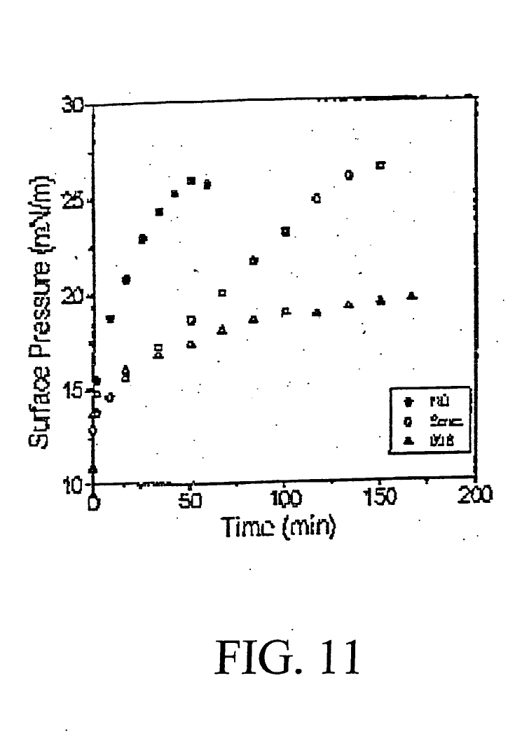

These data indicated that gp120 virotoxins can interact with both GalCer and GPR15/Bob on the surface of HT-29-D4 cells. In a recent study, it was shown that the interaction between GalCer and gp120 purified from HIV-1(IIIB) was very efficient, whereas gp120s from 89.6 and SEN isolates interacted very weakly (Hammache et al., 1998). The kinetics of this interaction are an important physicochemical parameter allowing the direct comparison of GalCer recognition by various gp120s. In the experiment shown in FIG. 11, gp120 purified from IIIB, SEN, or 89.6 was injected underneath a monomolecular film of GalCer at the air-water interface. The GalCer film mimics the topology of a GalCer raft as it is organized in the external leaflet of the plasma membrane. For this reason, the Langmuir film balance technique is one of the most sensitive and reliable for studying protein-glycolipid interactions (Maggio et al., 1994). Immediately after the addition of gp120(IIIB) in the aqueous subphase, an exponential increase in the surface pressure of the film was measured, and the equilibrium was reached after 45 minutes of incubation. The surface increase indicates a condensing effect of the gp120 on the lipidic film due to its interaction with GalCer molecules (Hammache et al., 1998). In contrast, the interaction with gp120(SEN) was very slow, linear rather than exponential, and the equilibrium was not reached even after 150 minutes of incubation. At this time however, the level of interaction indicated by the absolute increase of the surface pressure was identical to the one of gp120(IIIB). This indicates that the binding reaction between GalCer and gp120(SEN) requires a rearrangement of GalCer molecules that is not required for GalCer-gp120(IIIB) interaction. Finally, gp120(89.6) showed very little association with GalCer with a maximal surface pressure increase of less than 50% of the one measured for IIIB and SEN. Taken together, these data confirmed the lack of significant interaction between GalCer and gp120(89.6) and the high affinity of gp120(II13) for the lipid. The GalCer-binding capacity of gp120(SEN) was reevaluated and shown to occur much more slowly, but not less efficiently, than of gp120(IIIB).

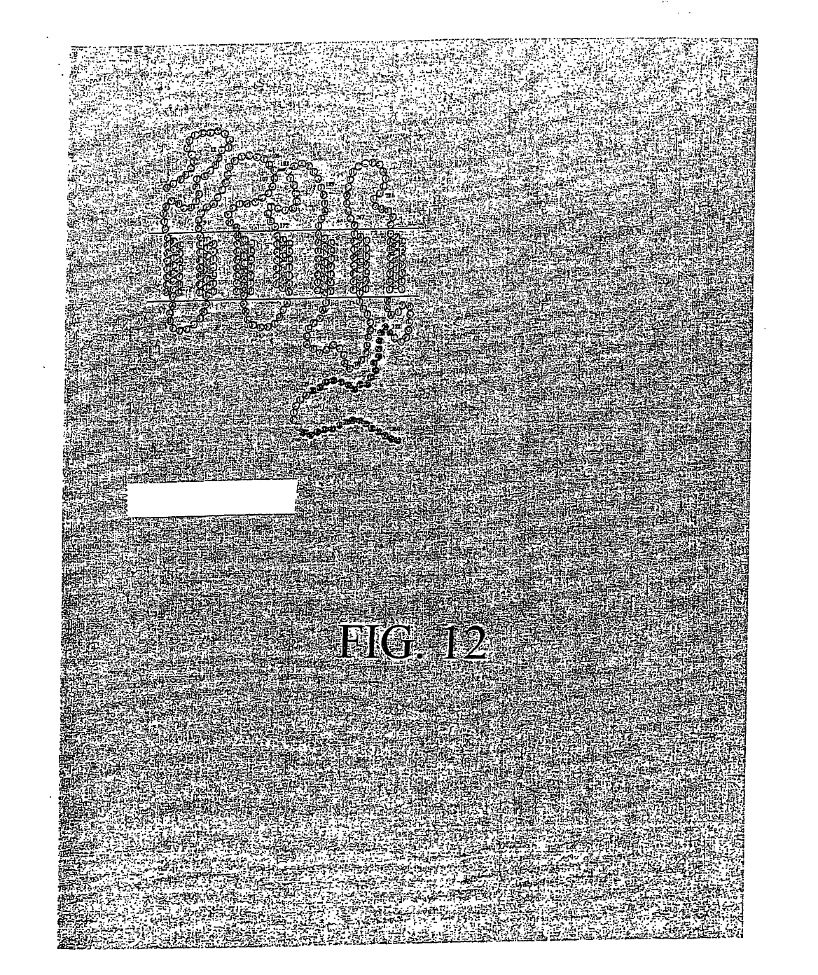

Several lines of evidence suggest that the HIV-1 fusion complex is assembled in glycolipid-cholesterol rich microdomains of the plasma membrane (Fantini et al., 2000b; Hug et al., 2000; Hammache et al., 1999; Manes et al., 2000; Puri et al., 1998; 1999). The role of these glycolipid rafts would be i) to bring the viral particles to an appropriate fusion coreceptor by lateral diffusion of the raft in the external leaflet of the plasma membrane (Hammache et al., 2000), and ii) to induce the conformational changes of HIV-1 envelope glycoproteins needed for exposing the fusion peptide (Hug et al., 2000). G protein transducer molecules are concentrated in rafts in various cell types including HT-29-D4 cells (Fantini et al., 2000a). Thus, GPR15/Bob was shown to associate with GalCer, an interaction which would allow the formation of a trimolecular complex involving a GalCer raft, gp120, and GPR15/Bob. The membrane topology and predicted orientation of GPR15/Bob is shown in FIG. 12. The synthetic peptides used for obtaining anti-GPR15/Bob antibodies are indicated. These peptides were injected, alone or in association, underneath a monomolecular film of GalCer prepared at the air-water interface. The maximal surface pressure increases measured after reaching equilibrium are shown in Table 5.

The external N-terminal peptide and the intracytoplasmic C-terminal peptides did not interact with GalCer. Peptides dervied from the second, third, and fourth external domains were found to interact with GalCer, suggesting that the receptor can be surrounded by several GalCer molecules belonging to distinct rafts. Thus, GPR15/Bob-induced fusion of these rafts not only increases the local concentration of gp120 in the vicinity of GPR15/Bob molecules, but also brings together the seven transmembrane domains receptor with raft-associated transducer molecules.

Thus, gp120 purified from selected HIV-1 strains can induce specific morphological and functional alterations in a model intestinal epithelium. These changes include microtubule disruption, appearance of intra- and inter-cellular lumina or cysts, perturbation of transepithelial resistance (TER), and decrease of sodium-dependent glucose absorption. All these symptoms are hallmarks of HIV-associated enteropathy (Clayton et al., 2001; Ehrenpreis et al., 1992; Kotler et al., 1984; Stockinann et al., 1998; Miller et al., 1988; Gillin et al., 1985; Patterson et al., 1993). The gp120s used were purified from laboratory and primary HIV-1 isolates produced in human peripheral blood mononuclear cells.

The intestinal tropism of HIV-1 has been previously established on the basis of infection of intestinal epithelial cell lines, chiefly HT-29 (Yahi et al., 1996; Trujillo et al., 2000). The conclusion of these studies was that those isolates able to infect intestinal cells in vitro recognized both GalCer and CXCR4 (Delezay et al., 1997a). HIV-1(IIIB), which interacts strongly with GalCer and uses CXCR4 as coreceptor to gain entry into target cells, can infect HT-29 cells. In contrast, HIV-1(89.6), a R5X4 isolate that can use CXCR4 but does not interact with GalCer, does not infect HT-29 cells (Hammache et al., 1998). HIV-1(SEN) is a R5 primary isolate that interacts weakly with GalCer and does not infect HT-29 cells (Hammache et al., 1998). Herein it was shown that the gp120s purified from 111B and SEN, but not from 89.6, induced marked alterations of TEER. Therefore, the capacity of gp120 to induce an HT-29 enteropathy is not correlated with the ability of the corresponding virus to infect HT-29 cells. These data strongly support the concept that HIV-associated gastrointestinal symptoms are due to a toxin-like effect of gp120 from specific isolates (e.g. IIIB and SEN) on intestinal epithelial cells. Since the TEER changes induced by these gp120s could be inhibited by anti-GPR15/Bob, this indicates this receptor is involved in the pathogenesis of HIV enteropathy. Strongly consistent with this, anti-CXCR4 antibodies that block HIV-1 infection of HT-29 cells (Delezay et al., 1997a; Trujillo et al., 2000) do not protect the cells from the toxic effects of gp120. Moreover, both anti-GalCer antibodies (which bind to the cell surface of intestinal cells) and a synthetic soluble analog of GalCer (which neutralizes soluble gp120), inhibited gp120-induced TEER changes. Taken together, these data indicate that HIV-1 enteropathy is caused by a subclass of gp120s which recognize both GalCer and GPR15/Bob on the surface of intestinal cells. The beneficial effects of antiretroviral therapies could be due to a blockade of HIV-1 replication in the lamina propria, resulting in a decrease in the concentration of gp120 virotoxins nearby the epithelium.

### 3. Example 3 AntiBob37 Neutralizing Antibody Inhibits or Delays Viral Infection

Peripheral blood mononuclear cells, isolated as disclosed in the examples, were incubated overnight in AIM-V medium (a serum-free artificial growth medium which does not induce a calcium flux, BRL/Gibco/Life Technologies) with 25 u/ml human recombinant IL-2, containing either 50 micrograms/ml antiBob37 or, as a control, 50 micrograms/ml normal rabbit gamma globulin (consisting almost entirely of IgG, Jackson Immunoresearch). After the overnight incubation, the cells were infected with either HIV LAI, HIV IIIB, or HIV MN at a multiplicity of infection in the range of 0.1 to 0.3, in the presence of 2 micrograms/ml polybrene. After 90 minutes, the virus was removed, and the culture medium was changed to RPMI 1640 supplemented with 10% fetal calf serum and 5 micrograms/ml phytohemaglutinin-P, but without the antiBob neutalizing antibodies. The culture supernates were collected and frozen daily from day 4 onward, and were tested for HIV p24 antigen by an Elisa method similar to that of Wehrly and Chesebro (Methods: A Companion to Methods in Enzymology 12: 288-293, 1997).

Conventionally, peripheral blood mononuclear cells are HIV infected when they are stimulated with fetal calf serum and, importantly, phytohemaglutinin, both of which, unlike the AIM-V medium, induce calcium fluxes. No effect with antiBob was seen under these conditions. Thus, the goal was to look for a difference in infection in resting lymphocytes. The antiBob37 treated cultures infected with HIV IIIB were consistently slower to grow than the controls and the total amount of virus produced was decreased. HIV IIIB was detected in 6 to 7 days and was maximal on day 8. If pretreated with antiBob37, infection was detected on day 7 to 8, and was maximal on day 9. Also, the peak HIV p24 protein content detected was reduced by approximately 68%. HIV MN-treated lymphocytes did not show detectable productive HIV infection for at least 10 days, whether or not they were treated with antiBob37.

The gp120 from HIV IIIB activates Bob, while the gp120 from HIV MN does not. These data indicate that Bob activation puts “resting” lymphocytes in a more HIV-infectious state and hastens their productive infection.

### 4. Example 4

### Simple Method of Detecting Bob-Activating Strains

Detecting calcium fluxes induced by HIV components has required the purification of the appropriate protein, which involves a biohazard associated with culture of large quantities of virus. HIV gp120 is also quite labile and hard to isolate in its active, undenatured state. Alternatives such as its production in transfected cells are laborious, particularly if done for a variety of strains, and do not eliminate the denaturation problem. HIV is traditionally grown in the presence of fetal bovine serum and phytohemaglutinin, both of which induce calcium fluxes which could interfere with a direct assay of culture supernates.

To overcome these problems, HIV was grown in cells which are initially in a traditional medium (RPMI 1640 with 10% fetal calf serum, 5 micrograms/ml phytohemaglutinin-P, and 20 u/ml IL-2, grown in this medium for 3 days prior to infection), then, 3 days after infection, switching the medium to AIM-V with 20 u/ml IL-2 but no serum or phytohemaglutinin. This medium does not induce calcium fluxes, allowing the direct testing of culture supernates for Bob activation in an appropriately transfected cell line. Furthermore, the Bob-transfected Ghost (3) cells are about 70 to 300-fold more sensitive to gp120-induced Bob activation than are the non-transfected cell line HT-29 or normal lymphocytes.

HIV LAI culture supernates obtained in this manner from the first day in which abundant p24 was detected in the culture supemates by ELISA (this is generally the 4^{th} or 5^{th} day after infection of activated lymphocytes) were inactivated with 0.12% Empigen BB (Calbiochem, La Jolla, Calif.), diluted in Locke's medium and tested with Ghost (3) Bob cells and the parent cell line. HIV p24 content of the supemate was 0.21 micrograms/ml. Calcium fluxes in the Ghost (3) Bob cells, but not the parent cell line, were detected in the 1:1,000, 1:10,000, and 1:100,000-fold dilutions. Similar testing with the MN strain (p24 content of 0.14 micrograms/ml) showed no significant calcium fluxes at 1:1,000, 1:10,000 or 1:100,000 dilutions.

### D. REFERENCES

1. Stockmann M, Fromm M, Schmitz H, Schmidt W, Riecken E O, Schulzke J D: Duodenal biopsies of HIV-infected patients with diarrhoea exhibit epithelial barrier defects but no active secretion. AIDS 1998,12:43-51.
2. Benhamou Y, Hilmarsdottir I, Desportes Livage I, Hoang C, Datry A, Danis M, Gentilini M, Opolon P: Association of lipid accumulation in small intestinal mucosa with decreased serum triglyceride and cholesterol levels in AIDS. Dig Dis Sci 1994,39:2163-9.
3. Ehrenpreis E D, Gulino S P, Patterson B K, Craig R M, Yokoo H, Atkinson A J, Jr.: Kinetics of D-xylose absorption in patients with human immunodeficiency virus enteropathy. Clin Pharmacol Ther 1991,49:632-40.
4. Ullrich R, Zeitz M, Heise W, L'Age M, Hoffken G, Riecken E O: Small intestinal structure and function in patients infected with human immunodeficiency virus (HIV): evidence for HIV-induced enteropathy. Ann Intern Med 1989,111:15-21.
5. Kotler DP, Shimada T, Snow G, Winson G, Chen W, Zhao M, Inada Y, Clayton F: Effect of combination antiretroviral therapy upon rectal mucosal HIV RNA burden and mononuclear cell apoptosis. AIDS 1998,12:597-604.
6. Dayanithi G, Yahi N, Baghdiguian S, Fantini J: Intracellular calcium release induced by human immunodeficiency virus type 1 (HIV-1) surface envelope glycoprotein in human intestinal epithelial cells: a putative mechanism for HIV-1 enteropathy. Cell Calcium 1995,18:9-18.
7. Delezay O, Yahi N, Tamalet C, Baghdiguian S, Boudier J A, Fantini J: Direct effect of type 1 human immunodeficiency virus (HIV-1) on intestinal epithelial cell differentiation: Relationship to HIV-1 enteropathy. Virology 1997,238:231-42.
8. Clayton F, Kapetanovic S K, Kotler D P: Enteric Microtubule Depolymerization in HIV Infection—A Possible Cause of HIV-Associated Enteropathy. AIDS 2001,15:123-4.
9. Glickman R M, Perrotto J L, Kirsch K: Intestinal lipoprotein formation: effect of cholchicine. Gastroenterology 1976,70:347-52.
10. Fradkin A, Yahav J, Diver Haber A, Zemer D, Jonas A: Colchicine enhances intestinal permeability in patients with familial Mediterranean fever. Eur J Clin Pharmacol 1996,51:241-5.
11. Dukes M N G, editor. Meyler's Side Effects of Drugs. 13 ed. Amsterdam: Elsevier; 1996.
12. Dwinell M B, Eckmann L, Leopard J D, Varki N M, Kagnoff M F: Chemokine receptor expression by human intestinal epithelial cells. Gastroenterology 1999,117:359-67.
13. Fox C H, Kotler D, Tierney A, Wilson C S, Fauci A S: Detection of HIV-1 RNA in the lamina propria of patients with AIDS and gastrointestinal disease. J Infect Dis 1989,159:467-71.
14. Deng H K, Unutmaz D, KewalRamani V N, Littman D R: Expression cloning of new receptors used by simian and human immunodeficiency viruses. Nature 1997,388:296-300.
15. Farzan M, Choe H, Martin K, Marcon L, Hofiann W, Karlsson G, Sun Y, Barrett P, Marchand N, Sullivan N, Gerard N, Gerard C, Sodroski J: Two orphan seven-transmembrane segment receptors which are expressed in CD4-positive cells support simian immunodeficiency virus infection. J Exp Med 1997,186:405-11.
16. Zhang L, He T, Huang Y, Chen Z, Guo Y, Wu S, Kunstman K J, Brown R C, Phair J P, Neumann A U, Ho D D, Wolinsky S M: Chemokine coreceptor usage by diverse primary isolates of human immunodeficiency virus type 1. J Virol 1998,72:9307-12.
17. Edinger A L, Hoffman T L, Sharron M, Lee B, O'Dowd B, Doms R W: Use of GPR1, GPR15, and STRL33 as coreceptors by diverse human immunodeficiency virus type 1 and simian immunodeficiency virus envelope proteins. Virology 1998,249:367-78.
18. Xiao L, Rudolph DL, Owen S M, Spira T J, Lal R B: Adaptation to promiscuous usage of CC and CXC-chemokine coreceptors in vivo correlates with HIV-1 disease progression. AIDS 1998,12:F13743.
19. Pohlmann S, Krumbiegel M, Kirchhoff F: Coreceptor usage of BOB/GPR15 and Bonzo/STRL33 by primary isolates of human immunodeficiency virus type 1. J Gen Virol 1999,80:1241-51.
20. Schneider J, Kaaden O, Copeland T D, Oroszlan S, Hunsmann G: Shedding and interspecies type sero-reactivity of the envelope glycopolypeptide gp120 of the human immunodeficiency virus. J Gen Virol 1986,67:2533-8.
21. Weissman D, Rabin R L, Arthos J, Rubbert A, Dybul M, Swofford R, Venkatesan S, Farber J M, Fauci A S: Macrophage-tropic HIV and SIV envelope proteins induce a signal through the CCR5 chemokine receptor. Nature 1997,389:981-5.
22. Herbein G, Mahlknecht U, Batliwalla F, Gregersen P, Pappas T, Butler J, O'Brien W A, Verdin E: Apoptosis of CD8+ T cells is mediated by macrophages through interaction of HIV gp120 with chemokine receptor CXCR4. Nature 1998,395:189-94.
23. Arthos J, Rubbert A, Rabin R L, Cicala C, Machado E, Wildt K, Hanbach M, Steenbeke T D, Swofford R, Farber J M, Fauci A S: CCR5 signal transduction in macrophages by human immunodeficiency virus and simian immunodeficiency virus envelopes. J Virol 2000,74:6418-24.
24. Oh S K, Cruikshank W W, Raina J, Blanchard G C, Adler W H, Walker J, Komfeld H: Identification of HIV-1 envelope glycoprotein in the serum of AIDS and ARC patients. J Acquir Immune Defic Syndr 1992,5:251-6.
25. Kuwada S K, Lund K A, Li X F, Cliften P, Amsler K, Opresko L K, Wiley H S: Differential signaling and regulation of apical vs. basolateral EGFR in polarized epithelial cells. Am J Physiol 1998,275:C1419-28.
26. Morgan T, Craven C, Ward K: Human spiral artery renin-angiotensin system. Hypertension 1998,32:683-7.
27. Momer A, Bjorndal A, Albert J, Kewalramani V N, Littman D R, Inoue R, Thorstensson R, Fenyo E M, Bjorling E: Primary human immunodeficiency virus type 2 (HIV-2) isolates, like HIV-1 isolates, frequently use CCR5 but show promiscuity in coreceptor usage. J Virol 1999,73:2343-9.
28. McLaurin J, Moscarello M A: Reactivity of two anti-galactosyl ceramide antibodies towards myelin basic protein. J Neurol Sci 1992,108:73-9.
29. O'Brien E T, Salmon E D, Erickson H P: How calcium causes microtubule depolymerization. Cell Motil Cytoskeleton 1997,36:125-35.
30. Waterman Storer C M, Salmon E: Positive feedback interactions between microtubule and actin dynamics during cell motility. Curr Opin Cell Biol 1999,11:61-7.
31. Madara J L, Pappenheimer J R: Structural basis for physiological regulation of paracellular pathways in intestinal epithelia. J Membr Biol 1987,100:149-64.
32. Baik S S, Doms R W, Doranz B J: HIV and SIV gp120 binding does not predict coreceptor function. Virology 1999,259:267-73.
33. Nye K E, Knox K A, Pinching A J: Lymphocytes from HIV-infected individuals show aberrant inositol polyphosphate metabolism which reverses after zidovudine therapy. AIDS 1991,5:413-7.
34. Iyengar S, Hildreth J E, Schwartz D H: Actin-dependent receptor colocalization required for human immunodeficiency virus entry into host cells. J Virol 1998,72:5251-5.
Achler, C., Filmer, D., Merte, C., and Drenckhahn, D. (1989). Role of microtubules in polarized delivery of apical membrane proteins to the brush border of the intestinal epithelium. J Cell Biol 109, 179-89.
Adachi, A., Koenig, S., Gendelman, H. E., Daugherty, D., Gattoni-Celli, S., Fauci, A. S., and Martin, M. A. (1987). Productive, persistent infection of human colorectal cell lines with human immunodeficiency virus. J Virol 61, 209-13.
Asmuth, D. M., Hammer, S. M., and Wanke, C. A. (1994). Physiological effects of HIV infection on human intestinal epithelial cells: an in vitro model for HIV enteropathy. AIDS 8, 205-11.
Barnett, S. W., Barboza, A., Wilcox, C. M., Forsmark, C. E., and Levy, J. A. (1991). Characterization of human immunodeficiency virus type 1 strains recovered from the bowel of infected individuals. Virology 182, 802-9.
Bartlett, J. G., Belistsos, P. C., and Sears, C. L. (1992). AIDS enteropathy. Clinical Infectious Diseases. 15, 726-735.
Benhamou, Y., Hilmarsdottir, I., Desportes-Livage, I., Hoang, C., Datry, A., Danis, M., Gentilini, M., and Opolon, P. (1994). Association of lipid accumulation in small intestinal mucosa with decreased serum triglyceride and cholesterol levels in AIDS. Dig Dis Sci 39, 2163-9.
Chenine, A. L., Matouskova, E., Sanchez, G., Reischig, J., Pavlikova, L., LeContel, C., Chermann, J. C., and Hirsch, I. (1998). Primary intestinal epithelial cells can be infected with laboratory-adapted strain HIV type I NDK but not with clinical primary isolates. AIDS Res Hum Retroviruses 14, 1235-8.
Clayton, F., Kapetanovic, S., and Kotler, D. P. (2001). Enteric microtubule depolymerization in HIV infection: a possible cause of HIV-associated enteropathy. AIDS 15, 123-4.
Collman, R., Balliet, J. W., Gregory, S. A., Friedman, H., Kolson, D. L., Nathanson, N., and Srinivasan, A. (1992). An infectious molecular clone of an unusual macrophage-tropic and highly cytopathic strain of human immunodeficiency virus type 1. J Virol 66, 7517-21.
Cook, D. G., Fantini, J., Spitalnik, S. L., and Gonzalez-Scarano, F. (1994). Binding of human immunodeficiency virus type I (HIV-1) gp120 to galactosylceramide (GalCer): relationship to the V3 loop. Virology 201, 206-14.
Cutz, E., Rhoads, J. M., Drumm, B., Sherman, P. M., Durie, P. R., and Forstner, G. G. (1989). Microvillus inclusion disease: an inherited defect of brush-border assembly and differentiation. N Engl J Med 320, 646-51.
Dayanithi, G., Yahi, N., Baghdiguian, S., and Fantini, J. (1995). Intracellular calcium release induced by human immunodeficiency virus type 1 (HIV-1) surface envelope glycoprotein in human intestinal epithelial cells: a putative mechanism for HIV-1 enteropathy. Cell Calcium 18, 9-18.
Delezay, O., Koch, N., Yahi, N., Hammache, D., Tourres, C., Tamalet, C., and Fantini, J. (1997a). Co-expression of CXCR4/fusin and galactosylceramide in the human intestinal epithelial cell line HT-29. Aids 11, 1311-8.
Delezay, O., Yahi, N., Tamalet, C., Baghdiguian, S., Boudier, J. A., and Fantini, J. (1997b). Direct effect of type 1 human immunodeficiency virus (HIV-1) on intestinal epithelial cell differentiation: relationship to HIV-1 enteropathy. Virology 238, 231-42.
Deng, H. K., Unutmaz, D., KewalRamani, V. N., and Littman, D. R. (1997). Expression cloning of new receptors used by simian and human immunodeficiency viruses. Nature 388, 296-300.
Denker, B. M., and Nigam, S. K. (1998). Molecular structure and assembly of the tight junction. Am J Physiol 274, F1-9.
Dwinell, M. B., Eckmann, L., Leopard, J. D., Varki, N. M., and Kagnoff, M. F. (1999). Chemokine receptor expression by human intestinal epithelial cells. Gastroenterology 117, 359-67.
Ehrenpreis, E. D., Ganger, D. R., Kochvar, G. T., Patterson, B. K., and Craig, R. M. (1992). D-xylose malabsorption: characteristic finding in patients with the AIDS wasting syndrome and chronic diarrhea. J Acquir Immune Defic Syndr 5, 1047-50.
Ehrenpreis, E. D., Gulino, S. P., Patterson, B. K., Craig, R. M., Yokoo, H., and Atkinson, A. J., Jr. (1991). Kinetics of D-xylose absorption in patients with human immunodeficiency virus enteropathy. Clin Pharmacol Ther 49, 632-40.
Fantini, J., Verrier, B., Marvaldi, J., and Mauchamp, J. (1989). In vitro differentiated HT 29-D4 clonal cell line generates leakproof and electrically active monolayers when cultured in porous-bottom culture dishes. Biol Cell 65, 163-9.
Fantini, J., Yahi, N., and Chermann, J. C. (1991). Human immunodeficiency virus can infect the apical and basolateral surfaces of human colonic epithelial cells. Proc Natl Acad Sci USA 88, 9297-301.
Fantini, J., Yahi, N., Baghdiguian, S., and Chermann, J. C. (1992). Human colon epithelial cells productively infected with human immunodeficiency virus show impaired differentiation and altered secretion. J Virol 66, 580-5.
Fantini, J., Hammache, D., Delezay, O., Yahi, N., Andre-Barres, C., Rico-Lattes, I., and Lattes, A. (1997). Synthetic soluble analogs of galactosylceramide (GalCer) bind to the V3 domain of HIV-1 gp120 and inhibit HIV-1-induced fusion and entry. J Biol Chem 272, 7245-52.
Fantini, J., Hammache, D., Delezay, O., Pieroni, G., Tamalet, C., and Yahi, N. (1998). Sulfatide inhibits HIV-1 entry into CD4−/CXCR4+cells. Virology 246, 211-20.
Fantini, J., Maresca, M., Hammache, D., Yahi, N., and Delezay, O. (2000a). Glycosphingolipid (GSL) microdomains as attachment platforms for host pathogens and their toxins on intestinal epithelial cells: activation of signal transduction pathways and perturbations of intestinal absorption and secretion. Glycoconj J17, 173-9.
Fantini, J., Hammache, D., Pieroni, G., and Yahi, N. (2000b). Role of glycosphingolipid microdomains in CD4-dependent HIV-1 fusion. Glycoconj J17, 199-204.
Fujita, E., Nishimura, S. I. (2000). Density dependent interaction of polymeric analogs of beta-galactosyl ceramide with gp120 of human immunodeficiency virus 1. Carbohydrate Lett. 4, 53-60.
Foudraine, N. A., Weverling, G. J., van Gool, T., Roos, M. T., de Wolf, F., Koopmans, P. P., van den Broek, P. J., Meenhorst, P. L., van Leeuwen, R., Lange, J. M., and Reiss, P. (1998). Improvement of chronic diarrhoea in patients with advanced HIV-1 infection during potent antiretroviral therapy. AIDS 12, 35-41.
Gerhard, R., Schmidt, G., Hofmann, F., and Aktories, K. (1998). Activation of Rho GTPases by Escherichia coli cytotoxic necrotizing factor 1 increases intestinal pemmeability in Caco-2 cells. Infect Immun 66, 5125-31.
Gillin, J. S., Shike, M., Alcock, N., Urmacher, S., Krown, S., Kurtz, R. C., Lightdale, C. J. and Winawer, S. J. (1985). Malabsorption and mucosal abnormalities of the small intestine in the acquired immunodeficiency syndrome. Ann. Intern. Med. 102, 619-624.
Glickman, R. M., Perrotto, J. L., and Kirsch, K. (1976). Intestinal lipoprotein formation: effect of cholchicine. Gastroenterology 70, 347-52.
Hammache, D., Pieroni, G., Yahi, N., Delezay, O., Koch, N., Lafont, H., Tamalet, C., and Fantini, J. (1998). Specific interaction of HIV-1 and HIV-2 surface envelope glycoproteins with monolayers of galactosylceramide and ganglioside GM3. J Biol Chem 273, 7967-71.
Hammache, D., Yahi, N., Maresca, M., Pieroni, G., and Fantini, J. (1999). Human erythrocyte glycosphingolipids as alternative cofactors for human immunodeficiency virus type 1 (HIV-1) entry: evidence for CD4-induced interactions between HIV-1 gp120 and reconstituted membrane microdomains of glycosphingolipids (Gb3 and GM3). J Virol 73, 5244-8.
Hopkins, A. M., Li, D., Mrsny, R. J., Walsh, S. V., and Nusrat, A. (2000). Modulation of tight junction function by G protein-coupled events. Adv Drug Deliv Rev 41, 329-40.
Hug, P., Lin, H. M., Korte, T., Xiao, X., Dimitrov, D. S., Wang, J. M., Puri, A., and Blumenthal, R. (2000). Glycosphingolipids promote entry of a broad range of human immunodeficiency virus type 1 isolates into cell lines expressing CD4, CXCR4, and/or CCR5. J. Virol 74, 6377-85.
Kotler, D. P., Gaetz, H. P., Lange, M., Klein, E. B., and Holt, P. R. (1984). Enteropathy associated with the acquired immunodeficiency syndrome. Ann. Intern. Med. 101, 421-428.
Kunzelmann, K. (1999). The cystic fibrosis transmembrane conductance regulator and its function in epithelial transport. Rev Physiol Biochem Pharmacol 137, 1-70.
Maggio, B. (1994). The surface behavior of glycosphingolipids in biomembranes: a new frontier of molecular ecology. Prog Biophys Mol Biol 62, 55-117.
Manes, S., del Real, G., Lacalle, R. A., Lucas, P., Gomez-Mouton, C., Sanchez-Palomino, S., Delgado, R., Alcami, J., Mira, E., and Martinez, A. C. (2000). Membrane raft microdomains mediate lateral assemblies required for HIV-1 infection. Embo 1, 190-6.
Meinild, A., Klaerke, D. A., Loo, D. D., Wright, E. M., and Zeuthen, T. (1998). The human Na+-glucose cotransporter is a molecular water pump. J Physiol 508, 15-21.
Miller, A. R., Griffin, G. E., Batman, P., Farquar, C., Forster, S. M., Pinching, A. J., and Harris, J. R. (1988). Jejunal mucosal architecture and fat absorption in male homosexuals infected with human immunodeficiency virus. Q J Med 69,1009-19.
Nelson, J. A., Wiley, C. A., Reynolds-Kohler C., Reese, C. E., Margaretten, W., and Levy, J. A. (1988). Human immunodefiency virus detected in bowel epithelium from patients with gastrointestinal symptoms. Lancet. i, 259-262.
Omary, M. B., Brenner, D. A., de Grandpre, L. Y., Roebuck, K. A., Richman, D. D., and Kagnoff, M. F. (1991). HIV-1 infection and expression in human colonic cells: infection and expression in CD4+ and CD4− cell lines. AIDS5, 275-81.
Patterson, B. K., Ehrenpreis E. D., and Yokoo, H. (1993). Focal enterocyte vacuolization. A new microscopic finding in the acquired immune deficiency wasting syndrome. Am. J. Clin. Pathol. 99, 24-27.
Pinto, M., Appay, M. D., Simon-Assman, P., Chevalier, G., Dracopoli, N., Fogh, J., and Zweibaum, A. (1982). Enterocytic differentiation of cultured human colon cancer cells by replacement of glucose by galactose in medium. Biol. Cell 44, 193-196.
Popovic, M., Samgadharan, M. G., Read, E., and Gallo, R. C. (1984). Detection, isolation, and continuous production of cytopathic retroviruses (HTLV-III) from patients with AIDS and pre-AIDS. Science 224, 497-500.
Puri, A., Hug, P., Jernigan, K., Barchi, J., Kim, H. Y., Hamilton, J., Wiels, J., Murray, G. J., Brady, R. O., and Blumenthal, R. (1998). The neutral glycosphingolipid globotriaosylceramide promotes fusion mediated by a CD4-dependent CXCR4-utilizing HIV type 1 envelope glycoprotein. Proc Natl Acad Sci USA 95, 14435-40.
Puri, A., Hug, P., Jernigan, K., Rose, P., and Blumenthal, R. (1999). Role of glycosphingolipids in HIV-1 entry: requirement of globotriosylceramide (Gb3) in CD4/CXCR4-dependent fusion. Biosci Rep 19, 317-25.
Stockmann, M., Frornm, M., Schmitz, H., Schmidt, W., Riecken, E. O., and Schulzke, J. D. (1998). Duodenal biopsies of HIV-infected patients with diarrhoea exhibit epithelial barrier defects but no active secretion. AIDS 12, 43-51.
Trujillo, J. R., Goletiani, N. V., Bosch, I., Kendrick, C., Rogers, R. A., Trujillo, E. B., Essex, M., and Brain, J. D. (2000). T-tropic sequence of the V3 loop is critical for HIV-1 infection of CXCR4-positive colonic HT-29 epithelial cells. J Acquir Immune Defic Syndr 25, 1-10.
Ullrich, R., Zeitz, M., Heise, W., L'age, M., Hoffken, G., and Riecken, E. O. (1989). Small intestinal structure and function in patients infected with human immunodeficiency virus (HIV). Evidence for HIV-induced enteropathy. Ann. Intern. Med. 111, 15-21.
Ullrich, R., Heise, W., Bergs, C., L'age, M., Riecken, E. O., and Zeitz, M. (1992). Effect of zidovudine treatment on the small intestinal mucosa in patients infected with the human immunodeficiency virus. Gastroenterology. 102, 1483-1492.
Veazey, R. S., and Lackner, A. (1998). The gastrointestinal tract and the pathogenesis of AIDS. AIDS 12, S35-S42.
Waterman-Storer, C. M., and Salmon, E. (1999). Positive feedback interactions between microtubule and actin dynamics during cell motility. Curr Opin Cell Biol 11, 61-7.
Yahi, N., Baghdiguian, S., Moreau, H., and Fantini, J. (1992). Galactosyl ceramide (or a closely related molecule) is the receptor for human immunodeficiency virus type 1 on human colon epithelial HT29 cells. J Virol 66, 4848-54.
Yahi, N., Sabatier, J. M., Baghdiguian, S., Gonzalez-Scarano, F., and Fantini, J. (1995). Synthetic multimeric peptides derived from the principal neutralization domain (V3 loop) of human immunodeficiency virus type 1 (HIV-1) gp120 bind to galactosylceramide and block HIV-1 infection in a human CD4-negative mucosal epithelial cell line. J Virol 69, 320-5.

Yahi, N., Ratner, L., Harouse, J. M., Gonzalez-Scarano, F., and Fantini, J. (1996). Genetic determinants controlling HIV-1 tropism for CD4⁻/GalCer⁺ human intestinal epithelial cells. Perspect. Drug Discov. Des. 5, 161-168.

| | |
|---|---|
| E. Sequences | |
| (SEQ ID NO: 1). | HAEDFARRRKRSVSL |
| (SEQ ID NO: 2). | DKEASLGLWRTGSFLCK |
| (SEQ ID NO: 3). | MDPEETSVYLDYYYATS |
| (SEQ ID NO: 4). | SGLRQEHYLPSAILQ |
| (SEQ ID NO: 5). | RELTLIDDKPYCAEKKAT |
| (SEQ ID NO: 6). | KNYDFGSSTETSDSHLTK |

| SEQ ID NO: 7 gp120 protein sequence | |
|---|---|
| MRVKEKYQHLWRWGWRWGTMLLGMLMICSATEKLWVTVYYGVPVWKEATTTLFCASDAKAYDTEVHNVWA | |
| THACVPTDPNPQEVVLVNVTENFNMWKNDMVEQMHEDIISLWDQSLKPCVKLTPLCVSLKCTDLKNDTNT | |
| NSSSGRMIMEKGEIKNCSFNISTSIRGKVQKEYAFFYKLDIIPIDNDTTSYTLTSCNTSVITQACPKVSF | |
| EPIPIHYCAPAGFAILKCNNKTFNGTGPCTNVSTVQCTHGIRPVVSTQLLLNGSLAEEEVVIRSANFTDN | |
| AKTIIVQLNQSVEINCTRPNNNTRKSIRIQRGPGRAFVTIGKIGNMRQAHCNISRAKWNNTLKQIDSKLR | |
| EQFGNNKTIIFKQSSGGDPEIVTHSFNCGGEFFYCNSTQLFNSTWFNSTWSTKGSNNTEGSDTITLPCRI | |
| KQIINMWQEVGKAMYAPPISGQIRCSSNITGLLLTRDGGNSNNESEIFRPGGGDMRDNWRSELYKYKVVK | |
| IEPLGVAPTKAKRRVVQREKR | |
| SEQ ID NO: 8 variant SEQ ID NO: 1 | |
| HAEDFARRRKRSISL | |
| SEQ ID NO: 9 Bob protein sequence | |
| MDPEETSVYLDYYYATSPNSDIRETHSHVPYTSVFLPVFYTAVFLTGVLGNLVLMGALHFKPGSR | |
| RLIDIFIINLAASDFIFLVTLPLWVDKEASLGLWRTGSFLCKGSSYMISVNMHCSVLLLTCMSVDRYLAI | |
| VWPVVSRKFRRTDCAYVVCASIWFISCLLGLPTLLSRELTLIDDKPYCAEKKATPIKLIWSLVALIFTFF | |
| VPLLSIVTCYCCIARKLCAHYQQSGKHNKKLKKSIKIIFIVVAAFLVSWLPFNTFKFLAIVSGLRQEHYL | |
| PSAILQLGMEVSGPLAFANSCVNPFIYYIFDSYIRRAIVHCLCPCLKNYDFGSSTETSDSHLTKALSTFI | |
| HAEDFARRRKRSVSL | |
| SEQ ID NO: 10 gp 120 DNA sequence HIVIIIB's gp120 (Nucleotides | |
| 6230 to 7746 fo full length, and the V3 loop is 7114 to 7218). | |
| ATGAGAGTGAAGGAGAAATATCAGCACTTGTGGAGATGGGGGTGGAGATGGGGCACCATGCTCCTTGGGA | |
| TGTTGATGATCTGTAGTGCTACAGAAAAATTGTGGGTCACAGTCTATTATGGGGTACCTGTGTGGAAGGA | |
| AGCAACCACCACTCTATTTTGTGCATCAGATGCTAAAGCATATGATACAGAGGTACATAATGTTTGGGCC | |
| ACACATGCCTGTGTACCCACAGACCCCAACCCACAAGAAGTAGTATTGGTAAATGTGACAGAAAATTTTA | |
| ACATGTGGAAAAATGACATGGTAGAACAGATGCATGAGGATATAATCAGTTTATGGGATCAAAGCCTAAA | |
| CCATGTGTAAAATTAACCCCACTCTGTGTTAGT | |
| TTAAAGTGCACTGATTTGAAGAATGATACTAATACCAATAGTAGTAGCGGGAGAATGATAATGGAGAAAG | |
| GAGAGATAAAAAACTGCTCTTTCAATATCAGCACAAGCATAAGAGGTAAGGTGCAGAAAGAATATGCATT | |
| TTTTTATAAACTTGATATAATACGAATAGATAATGATACTACCAGCTATACGTTGACAAGTTGTAACACC | |
| TGAGTCATTACACAGGCCTGTCCAAAGGTATCCTTTGAGCCAATCCCATACATTATTGTGCCCCGGCTGG | |
| TTTTGCGATTCTAAAATGTAATAATAAGACGTTCAATGGAACAGGACCATGTACAAATGTCAGCACAGTA | |
| CAATGTAGACATGGATTAGGCCAGTAGTATCAACTCAACTGCTGTTAAATGGCAGTCTGGCAGAAGAAGA | |
| GGTAGTAATTAGATCTGCCAATTTCACAGACAATGCTAAAACCATAATAGTACAGCTGAACCAATCTGTA | |
| GAAATTAATTGTACAAGACCCAACAACAATACAAGAAAAAGTATCCGTATCCAGAGAGGACCAGGGAGAG | |
| CATTTGTTACAATAGGAATAAAAATAGGAAATATGAGACAAGCACATTGTAACATTAGTAGAGCAAAATG | |
| GAATAACACTTTAAAACAGATAGATAGCAAATTAAGAGAACAATTTGGAAATAATAAAACAATAATCTTT | |
| AAGCAGTCCTCAGGAGGGGACCCAGAAAGTTGTAACGCACAGTTTTAATTGTGGAGGGGAATTTTTCTAC | |
| TGTAATTCAACACAACTGTTTAATAGTACTTGGTTTAATAGTACTTGGAGTACTAAAGGGTCAAATAACA | |
| CTGAAGGAAGTGACACAATCACCCTCCCATGCAGAATAAAACAAATTATAAACATGTGGCAGGAAGTAGG | |
| AAAAGCAATGTATGCCCCTCCCATCAGTGGACAAATTAGATGTTCATCAAATATTACAGGGCTGCTATTA | |
| ACAAGAGATGGTGGTAATAGCAACAATGAGTCCGAGATCTTCAGACCTGGAGGAGGAGATATGAGGGACA | |
| ATTGGAGAAGTGAATTATATAAATATAAAGTAGTAAAAATTGAACCATTAGGAGTAGCACCCACCCAAGG | |
| CAAAGAGAAGAGTGGTGCAGAGAGAAAAAAGA | |
| SEQ ID NO: 11 Bob DNA sequence | |
| 1 | atggacccag aagaaacttc agtttatttg gattattact atgctacgag cccaaactct |
| 61 | gacatcaggg agacccactc ccatgttcct tacacctctg tcttccttcc agtcttttac |
| 121 | acagctgtgt tcctgactgg agtgctgggg aaccttgttc tcatgggagc gttgcatttc |
| 181 | aaacccggca gccgaagact gatcgacatc tttatcatca atctggctgc ctctgacttc |
| 241 | atttttcttg tcacattgcc tctctgggtg gataaagaag catctctagg actgtggagg |
| 301 | acgggctcct tcctgtgcaa agggagctcc tacatgatct ccgtcaatat gcactgcagt |
| 361 | gtcctcctgc tcacttgcat gagtgttgac cgctacctgg ccattgtgtg gccagtcgta |
| 421 | tccaggaaat tcagaaggac agactgtgca tatgtagtct gtgccagcat ctggtttatc |
| 481 | tcctgcctgc tggggttgcc tactcttctg tccagggagc tcacgctgat tgatgataag |
| 541 | ccatactgtg cagagaaaaa ggcaactcca attaaactca tatggtccct ggtggcctta |
| 601 | attttcacct tttttgtccc tttgttgagc attgtgacct gctactgttg cattgcaagg |
| 661 | aagctgtgtg cccattacca gcaatcagga aagcacaaca aaaagctgaa gaaatctata |
| 721 | aagatcatct ttattgtcgt ggcagccttt cttgtctcct ggctgccctt caatactttc |
| 781 | aagttcctgg ccattgtctc tgggttgcgg caagaacact atttaccctc agctattctt |
| 841 | cagcttggta tggaggtgag tggacccttg gcatttgcca acagctgtgt caaccctttc |
| 901 | atttactata tcttcgacag ctacatccgc cgggccattg tccactgctt gtgcccttgc |
| 961 | ctgaaaaact atgactttgg gagtagcact gagacatcag atagtcacct cactaaggct |
| 1021 | ctctccacct tcattcatgc agaagatttt gccaggagga ggaagaggtc tgtgtcactc |
| 1081 | taa |
| SEQ ID NO: 12 gp120 V3 sequence for isolate 89.6 | |
| CTRPNNNTRRRLSIGPGRAFYARRNIIGDIRQAHC | |
| SEQ ID NO: 13 gp120 V3 sequence for isolate SEN, | |
| CTRPNNNTRKRISMGPGRVFYTTKDIVGDIRQAHC | |
| SEQ ID NO: 14 Full length HIVIIIb genome | |
| | ggtctctcnn gttagaccag atttgagcct ggnagctctc tggctaacta gggaacccac |
| 61 | tgcttaagcc tcaataaagc ttgccttgag tgcttcaagt agtgtgtgcc cgtctgttgt |
| 121 | gtgactctgg taactagaga tccctcagac ccttttagtc agtgtggaaa atctctagca |
| 181 | gtggcgcccg aacagggact tgaaagcgaa agggaaacca gaggagctct ctcgacgcag |
| 241 | gactcggctt gctgaagcgc gcacggcaag aggcgagggg aggcgactgg tgagtacgcc |
| 301 | aaaaattttg actagcggag gctagaagga gagagatggg tgcgagagcg tcagtattaa |
| 361 | gcgggggaga attagatcga tgggaagaaa ttcggttaag gccaggggga aagaaaaaat |
| 421 | ataaattaaa acatatagta tgggcaagca gggagctaga acgattcgca gttaatcctg |
| 481 | gcctgttaga aacatcagaa ggctgtagac aaatactggg acagctacaa ccatcccttc |
| 541 | agacaggatc agaagaactt agatcattat ataatacagt agcaaccctc tattgtgtgc |
| 601 | atcaaaggat agagataaaa gacaccaagg aagctttaga caagatagag gaagagcaaa |
| 661 | acaaaagtaa gaaaaaagca cagcaagcag cagctgacac aggacacagc agccaggtca |
| 721 | gccaaaatta ccctatagtg cagaacatcc aggggcaaat ggtacatcag gccatatcac |
| 781 | ctagaacttt aaatgcatgg gtaaaagtag tagaagagaa ggctttcagc ccagaagtga |
| 841 | tacccatgtt ttcagcatta tcagaaggag ccaccccaca agatttaaac accatgctaa |
| 901 | acacagtggg gggacatcaa gcagccatgc aaatgttaaa agagaccatc aatgaggaag |
| 961 | ctgcagaatg ggatagagtg catccagtgc atgcagggcc tattgcacca ggccagatga |
| 1021 | gagaaccaag gggaagtgac atagcaggaa ctactagtac ccttcaggaa caaataggat |
| 1081 | ggatgacaaa taatccacct atcccagtag gagaaattta taaaagatgg ataatcctgg |
| 1141 | gattaaataa aatagtaaga atgtatagcc ctaccagcat tctggacata agacaaggac |
| 1201 | caaaagaacc ctttagagac tatgtagacc ggttctataa aactctaaga gccgagcaag |
| 1261 | cttcacagga ggtaaaaaat tggatgacag aaaccttgtt ggtccaaaat gcgaacccag |
| 1321 | attgtaagac tattttaaaa gcattgggac cagcagctac actagaagaa atgatgacag |
| 1381 | catgtcaggg agtgggagga cccggccata aggcaagagt tttggctgaa gcaatgagcc |
| 1441 | aagtaacaaa ttcagctacc ataatgatgc aaagaggcaa ttttaggaac caaagaaaga |
| 1501 | ttgttaagtg tttcaattgt ggcaaagaag ggcacatagc cagaaattgc agggccccta |
| 1561 | ggaaaaaggg ctgttggaaa tgtggaaagg aaggacacca aatgaaagat tgtactgaga |
| 1621 | gacaggctaa ttttttaggg aagatctggc cttcctacaa gggaaggcca gggaattttc |
| 1681 | ttcagagcag accagagcca acagccccac cagaagagag cttcaggtct ggggtagaga |
| 1741 | caacaactcc ctctcagaag caggagccga tagacaagga actgtatcct ttaacttccc |
| 1801 | tcagatcact ctttggcaac gacccctcgt cacaataaag ataggggggc aactaaagga |
| 1861 | agctctatta gatacaggag cagatgatac agtattagaa gaaatgagtt tgccaggaag |
| 1921 | atggaaacca aaaatgatag ggggaattgg aggttttatc aaagtaagac agtatgatca |
| 1981 | gatactcata gaaatctgtg gacataaagc tataggtaca gtattagtag gacctacacc |
| 2041 | tgtcaacata attggaagaa atctgttgac tcagattggt tgcactttaa attttcccat |
| 2101 | tagtcctatt gaaactgtac cagtaaaatt aaagccagga atggatggcc caaaagttaa |
| 2161 | acaatggcca ttgacagaag aaaaaataaa agcattagta gaaatttgta cagaaatgga |
| 2221 | aaaggaaggg aaaatttcaa aaattgggcc tgaaaatcca tacaatactc cagtatttgc |
| 2281 | cataaagaaa aaagacagta ctaaatggag aaaattagta gatttcagag aacttaataa |
| 2341 | gagaactcaa gacttctggg aagttcaatt aggaatacca catcccgcag ggttaaaaaa |
| 2401 | gaaaaaatca gtaacagtac tggatgtggg tgatgcatat ttttcagttc ccttagatga |
| 2461 | agacttcagg aagtatactg catttaccat acctagtata aacaatgaga caccagggat |
| 2521 | tagatatcag tacaatgtgc ttccacaggg atggaaagga tcaccagcaa tattccaaag |
| 2581 | tagcatgaca aaaatcttag agccttttag aaaacaaaat ccagacatag ttatctatca |
| 2641 | atacatggat gatttgtatg taggatctga cttagaaata gggcagcata gaacaaaaat |
| 2701 | agaggagctg agacaacatc tgttgaggtg gggacttacc acaccagaca aaaaacatca |
| 2761 | gaaagaacct ccattccttt ggatgggtta tgaactccat cctgataaat ggacagtaca |
| 2821 | gcctatagtg ctgccagaaa aagacagctg gactgtcaat gacatacaga agttagtggg |
| 2881 | aaaattgaat tgggcaagtc agatttaccc agggattaaa gtaaggcaat tatgtaaact |
| 2941 | ccttagagga accaaagcac taacagaagt aataccacta acagaagaag cagagctaga |
| 3001 | actggcagaa aacagagaga ttctaaaaga accagtacat ggagtgtatt atgacccatc |
| 3061 | aaaagactta atagcagaaa tacagaagca ggggcaaggc caatggacat atcaaattta |
| 3121 | tcaagagcca tttaaaaatc tgaaaacagg aaaatatgca agaacgaggg gtgcccacac |
| 3181 | taatgatgta aaacaattaa cagaggcagt gcaaaaaata accacagaaa gcatagtaat |
| 3241 | atggggaaag actcctaaat ttaaactacc catacaaaag gaaacatggg aaacatggtg |
| 3301 | gacagagtat tggcaagcca cctggattcc tgagtgggag tttgtcaata cccctccttt |
| 3361 | agtgaaatta tggtaccagt tagagaaaga acccatagta ggagcagaaa cgttctatgt |
| 3421 | agatggggca gctagcaggg agactaaatt aggaaaagca ggatatgtta ctaatagagg |
| 3481 | aagacaaaaa gttgtcaccc taactgacac aacaaatcag aagactgagt tacaagcaat |
| 3541 | tcatctagct ttgcaggatt cgggattaga agtaaatata gtaacagact cacaatatgc |
| 3601 | attaggaatc attcaagcac aaccagataa aagtgaatca gagttagtca atcaaataat |
| 3661 | agagcagtta ataaaaaagg aaaaggtcta tctggcatgg gtaccagcac acaaaggaat |
| 3721 | tggaggaaat gaacaagtag ataaattagt cagtgctgga atcaggaaag tactattttt |
| 3781 | agatggaata gataaggccc aagatgaaca tgagaaatat cacagtaatt ggagagcaat |
| 3841 | ggctagtgat tttaacctgc cacctgtagt agcaaaagaa atagtagcca gctgtgataa |
| 3901 | atgtcagcta aaaggagaag ccatgcatgg acaagtagac tgtagtccag gaatatggca |
| 3961 | actagattgt acacatttag aaggaaaagt tatcctggta gcagttcatg tagccagtgg |
| 4021 | atatatagaa gcagaagtta ttccagcaga aacagggcag gaaacagcat actttctttt |
| 4081 | aaaattagca ggaagatggc cagtaaaaac aatacataca gacaatggca gcaatttcac |
| 4141 | cagtactacg gttaaggccg cctgttggtg ggcgggaatc aagcaggaat ttggaattcc |
| 4201 | ctacaatccc caaagtcaag gagtagtaga atctatgaat aaagaattaa agaaaattat |
| 4261 | aggccaggta agagatcagg ctgaacatct taagacagca gtacaaatgg cagtattcat |
| 4321 | ccacaatttt aaaagaaaag gggggattgg ggggtacagt gcaggggaaa gaatagtaga |
| 4381 | cataatagca acagacatac aaactaaaga attacaaaaa caaattacaa aaattcaaaa |
| 4441 | ttttcgggtt tattacaggg acagcagaga tccactttgg aaaggaccag caaagctcct |
| 4501 | ctggaaaggt gaaggggcag tagtaataca agataatagt gacataaaag tagtgccaag |
| 4561 | aagaaaagca aagatcatta gggattatgg aaaacagatg gcaggtgatg attgtgtggc |
| 4621 | aagtagacag gatgaggatt agaacatgga aaagtttagt aaaacaccat atgtatgttt |
| 4681 | cagggaaagc taggggatgg ttttatagac atcactatga aagccctcat ccaagaataa |
| 4741 | gttcagaagt acacatccca ctaggggatg ctagattggt aataacaaca tattggggtc |
| 4801 | tgcatacagg agaaagagac tggcatctgg gtcagggagt ctccatagaa tggaggaaaa |
| 4861 | agagatatag cacacaagta gaccctgaac tagcagacca actaattcat ctgtattact |
| 4921 | ttgactgttt ttcagactct gctataagaa aggccttatt aggacatata gttagcccta |
| 4981 | ggtgtgaata tcaagcagga cataacaagg taggatctct acaatacttg gcactagcag |
| 5041 | cattaataac accaaaaaag ataaagccac ctttgcctag tgttacgaaa ctgacagagg |
| 5101 | atagatggaa caagccccag aagaccaagg gccacagagg gagccacaca atgaatggac |
| 5161 | actagagctt ttagaggagc ttaagaatga agctgttaga cattttccta ggatttggct |
| 5221 | ccatggctta gggcaacata tctatgaaac ttatggggat acttgggcag gagtggaagc |
| 5281 | cataataaga attctgcaac aactgctgtt tatccatttc agaattgggt gtcgacatag |
| 5341 | cagaataggc gttactcaac agaggagagc aagaaatgga gccagtagat cctagactag |
| 5401 | agccctggaa gcatccagga agtcagccta aaactgcttg taccacttgc tattgtaaaa |
| 5461 | agtgttgctt tcattgccaa gtttgtttca caacaaaagc cttaggcatc tcctatggca |
| 5521 | ngaagaagcg gagacagcga cgaanacctc ctcaaggcag tcagactcat caagtttctc |
| 5581 | tatcaaagca gtaagtagta catgtaatgc aacctataca aatagcaata gcagcattag |
| 5641 | tagtagcaat aataatagca atagttgtgt ggtccatagt aatcatagaa tataggaaaa |
| 5701 | tattaagaca aagaaaaata gacaggttaa ttgatagact aatagaaaga gcagaagaca |
| 5761 | gtggcaatga gagtgaagga gaaatatcag cacttgtgga gatgggggtg gaaatggggc |
| 5821 | accatgctcc ttgggatatt gatgatctgt agtgctacag aaaaattgtg ggtcacagtc |
| 5881 | tattatgggg tacctgtgtg gaaggaagca accaccactc tattttgtgc atcagatgct |
| 5941 | aaagcatatg atacagaggt acataatgtt tgggccacac atgcctgtgt acccacagac |
| 6001 | cccaacccac aagaagtagt attggtaaat gtgacagaaa attttaacat gtggaaaaat |
| 6061 | gacatggtag aacagatgca tgaggatata atcagtttat gggatcaaag cctaaagcca |
| 6121 | tgtgtaaaat taaccccact ctgtgttagt ttaaagtgca ctgatttggg gaatgctact |
| 6181 | aataccaata gtagtaatac caatagtagt agcggggaaa tgatgatgga gaaaggagag |
| 6241 | ataaaaaact gctctttcaa tatcagcaca agcataagag gtaaggtgca gaaagaatat |
| 6301 | gcattttttt ataaacttga tataatacca atagataatg atactaccag ctatacgttg |
| 6361 | acaagttgta acacctcagt cattacacag gcctgtccaa aggtatcctt tgagccaatt |
| 6421 | cccatacatt attgtgcccc ggctggtttt gcgattctaa aatgtaataa taagacgttc |
| 6481 | aatggaacag gaccatgtac aaatgtcagc acagtacaat gtacacatgg aattaggcca |
| 6541 | gtagtatcaa ctcaactgct gttgaatggc agtctagcag aagaagaggt agtaattaga |
| 6601 | tctgccaatt tcacagacaa tgctaaaacc ataatagtac agctgaacca atctgtagaa |
| 6661 | attaattgta caagacccaa caacaataca agaaaaagta tccgtatcca gaggggacca |
| 6721 | gggagagcat ttgttacaat aggaaaaata ggaaatatga gacaagcaca ttgtaacatt |
| 6781 | agtagagcaa aatggaatgc cactttaaaa cagatagcta gcaaattaag agaacaattt |
| 6841 | ggaaataata aaacaataat ctttaagcaa tcctcaggag gggacccaga aattgtaacg |
| 6901 | cacagtttta attgtggagg ggaatttttc tactgtaatt caacacaact gtttaatagt |
| 6961 | acttggttta atagtacttg gagtactgaa gggtcaaata acactgaagg aagtgacaca |
| 7021 | atcacactcc catgcagaat aaaacaattt ataaacatgt ggcaggaagt aggaaaagca |
| 7081 | atgtatgccc ctcccatcag cggacaaatt agatgttcat caaatattac agggctgcta |
| 7141 | ttaacaagag atggtggtaa taacaacaat gggtccgaga tcttcagacc tggaggagga |
| 7201 | gatatgaggg acaattggag aagtgaatta tataaatata aagtagtaaa aattgaacca |
| 7261 | ttaggagtag cacccaccaa ggcaaagaga agagtggtgc agagagaaaa aagagcagtg |
| 7321 | ggaataggag ctttgttcct tgggttcttg ggagcagcag gaagcactat gggcgcacgg |
| 7381 | tcaatgacgc tgacggtaca ggccagacaa ttattgtctg gtatagtgca gcagcagaac |
| 7441 | aatttgctga gggctattga ggcgcaacag catctgttgc aactcacagt ctggggcatc |
| 7501 | aagcagctcc aggcaagaat cctggctgtg gaaagatacc taaaggatca acagctcctg |
| 7561 | gggatttggg gttgctctgg aaaactcatt tgcaccactg ctgtgccttg gaatgctagt |
| 7621 | tggagtaata aatctctgga acagatttgg aataacatga cctggatgga gtgggacaga |
| 7681 | gaaattaaca attacacaag cttaatacat tccttaattg aagaatcgca aaaccagcaa |
| 7741 | gaaaagaatg aacaagaatt attggaatta gataaatggg caagtttgtg gaattggttt |
| 7801 | aacataacaa attggctgtg gtatataaaa atattcataa tgatagtagg aggcttggta |
| 7861 | ggtttaagaa tagtttttgc tgtactttct atagtgaata gagttaggca gggatattca |
| 7921 | ccattatcgt ttcagaccca cctcccaacc ccgaggggac ccgacaggcc cgaaggaata |
| 7981 | gaagaagaag gtggagagag agacagagac agatccattc gattagtgaa cggatcctta |
| 8041 | gcacttatct gggacgatct gcggagcctg tgcctcttca gctaccaccg cttgagagac |
| 8101 | ttactcttga ttgtaacgag gattgtggaa cttctgggac gcagggggtg ggaagccctc |
| 8161 | aaatattggt ggaatctcct acagtattgg agtcaggaac taaagaatag tgctgttagc |
| 8221 | ttgctcaatg ccacagccat agcagtagct gaggggacag atagggttat agaagtagta |
| 8281 | caaggagctt gtagagctat tcgccacata cctagaagaa taagacaggg cttggaaagg |
| 8341 | attttgctat aagatgggtg gcaagtggtc aaaaagtagt gtggttggat ggcctactgt |
| 8401 | aagggaaaga atgagacgag ctgagccagc agcagatggg gtgggagcag catctcgaga |
| 8461 | cctggaaaaa catggagcaa tcacaagtag caatacagca gctaccaatg ctgcttgtgc |
| 8521 | ctggctagaa gcacaagagg aggaggaggt gggttttcca gtcacacctc aggtaccttt |
| 8581 | aagaccaatg acttacaagg cagctgtaga tcttagccac tttttaaaag aaaagggggg |
| 8641 | actggaaggg ctaattcact cccaacgaag acaagatatc cttgatctgt ggatctacca |
| 8701 | cacacaaggc tacttccctg attggcagaa ctacacacca gggccagggg tcagatatcc |
| 8761 | actgaccttt ggatggtgct acaagctagt accagttgag ccagataagg tagaagaggc |
| 8821 | caataaagga gagaacacca gcttgttaca ccctgtgagc ctgcatggaa tggatgaccc |
| 8881 | tgagagagaa gtgttagagt ggaggtttga cagccgccta gcatttcatc acgtggcccg |
| 8941 | agagctgcat ccggagtact tcaagaactg ctgacatcga gcttgctaca agggactttc |
| 9001 | cgctggggac tttccaggga ggcgtggcct ggnngggact ggggagtggc gagccctcag |
| 9061 | atgctgcata taagcagctg ctttttgcct gtactgggtc tctctggtta gaccagattt |
| 9121 | gagcctggga gctctctggc taactaggga acccactgct taagcctcaa taaagcttgc |
| 9181 | cttgagtgct tca |

| | |
|---|---|
| SEQ ID NO: 15 | GPGRAF |
| SEQ ID NO: 16 | IQRGPG |
| SEQ ID NO: 17: | GPGRAFVTI |

1. A composition for reducing an interaction between Bob and gp120 comprising a Bob inhibitor that binds a region of Bob, wherein the region of Bob comprises amino acids, 1-33, 91-107, 172-189, or 267-281 of SEQ ID NO 9.

2. A composition for reducing an interaction between Bob and gp120 comprising a Bob inhibitor that binds a region of Bob, and wherein the substance binds Bob preferentially over galactosyl ceramide.

3. The composition of claim 2, wherein the Bob inhibitor does not bind nerve cells.

4. A composition for reducing an interaction between Bob and gp120 comprising a substance that interacts with the N-terminal sequence of the 1^{st} loop or the 1^{st} extracellular loop domains of Bob.

5. A composition for reducing the interaction between Bob and gp120 comprising a substance that binds a peptide having a sequence with at least 80% identity to SEQ ID NO:2, SEQ ID NO:3, SEQ ID NO:4, or SEQ ID NO:5.

6. A composition for reducing the interaction between Bob and gp120 comprising a substance that binds a peptide having a sequence with at least 80% identity to SEQ ID NO:2 or SEQ ID NO:3.

7. The composition of claim 6, wherein the composition binds the peptide with a Kd of less than or equal to 10⁻⁶, 10⁻⁶ M, 10⁻⁷ M, 10⁻⁸ M, 10⁻⁹ M, 10⁻¹⁰ M, 10⁻¹¹ M, or 10⁻¹² M.

8. The composition of claim 6, wherein the composition preferentially binds Bob over galactose-ceramide.

9. The composition of claim 6 wherein the composition binds Bob with an affinity at least 5, 10, 25, 50, 75, 100, 125, 150, or 200 fold better than galactosyl ceramide.

10. The composition of claim 6, wherein the composition is an antibody or antibody fragment.

11. The composition of claim 10, wherein the antibody or antibody fragment is a polyclonal antibody.

12. The composition of claim 11, wherein the antibody or antibody fragment is Bob 37.

13. The composition of claim 10, wherein the antibody or antibody fragment is a monoclonal antibody.

14. The composition of claim 13, wherein the antibody or antibody fragment is humanized.

15. The composition of claim 6, wherein the composition inhibits gp120 activation of a lymphocyte, macrophage, intestinal epithelial cell, renal tubular cell, renal glomerular epithelial cell, hepatocyte, prostatic epithelial cell, or germinal epithelium of the testis or sperm.

16. The composition of claim 15, wherein the activation occurs at gp120 concentrations below 0.15 nM.

17. The composition of claim 6, wherein the composition inhibits gp120 activation on the basolateral surface of enteric epithlium.

18. The composition of claim 6, wherein the composition inhibits the gp120 induced signaling in cells that express Bob.

19. The composition of claim 18, wherein the signaling is calcium signaling.

20. The composition of claim 6, wherein the composition inhibits inositol triphosphate activation by gp120.

21. The composition of claim 6, wherein the composition delays the productive infection of CD4 lymphocytes or macrophages.

22. A cell comprising the composition of claim 6.

23. An animal comprising the cell of claim 22, wherein the animal is not a human.

24. An animal comprising the composition of claim 6, wherein the animal is not a human.

25. The composition of claim 6, wherein the composition comprises an aptamer, peptide, or peptide memetic.

26. The composition of claim 6, wherein the composition further comprises a pharmaceutical acceptable carrier.

27. A composition comprising a monoclonal antibody or antibody fragment, wherein the monoclonal antibody or antibody fragment binds Bob, and wherein the monoclonal antibody is produced by collecting the secreted material of a hybridoma cell producing the antibody, wherein the antibody binds Bob.

28. The composition of claim 27, further comprising isolating the DNA encoding the monoclonal antibody or antibody fragment and producing the monoclonal antibody or antibody fragment using recombinant biotechnology techniques.

29. A composition comprising a monoclonal antibody or antibody fragment, wherein the monoclonal antibody or antibody fragment binds Bob, and wherein the monoclonal antibody is produced by immunizing a lymphocyte with a peptide comprising the sequence set forth in SEQ ID NO:1, SEQ ID NO:2, SEQ ID NO:3, SEQ ID NO:4, SEQ ID NO:5, or SEQ ID NO:6, isolating a cell producing the antibody that binds the peptide, fusing the cell with an immortal cell line forming a hybridoma, and collecting the secreted material of the hybridoma cell producing the antibody.

30. The composition of claim 29, wherein immunizing a lymphocyte occurs in a mammal.

31. The composition of claim 29, wherein the cell comprises a peripheral blood lymphocyte, spleen cell, or lymph node cell.

32. The composition of claim 29, wherein the cell line comprises a transformed mammalian cell line.

33. The composition of claim 32, wherein the cell line comprises a myeloma cell line.

34. The composition of claim 29, wherein the hybridoma cell is maintained as an ascyte in a mammal.

35. A method for producing an antibody to Bob comprising immunizing a mammal with a peptide comprising the sequence set forth in SEQ ID NO:1, SEQ ID NO:2, SEQ ID NO:3, SEQ ID NO:4, SEQ ID NO:5, or SEQ ID NO:6 and collecting the sera of the mammal.

36. A method for producing a monoclonal antibody to Bob comprising immunizing a lymphocyte with a peptide comprising the sequence set forth in SEQ ID NO:1, SEQ ID NO:2, SEQ ID NO:3, SEQ ID NO:4, SEQ ID NO:5, or SEQ ID NO:6, isolating a cell producing the antibody that binds the peptide, fusing the cell with an immortal cell line forming a hybridoma, and collecting the secreted material of the hybridoma cell producing the antibody.

37. The method of claim 36, wherein immunizing a lymphocyte occurs in a mammal.

38. The method of claim 37, wherein the cell comprises a peripheral blood lymphocyte, spleen cell, or lymph node cell.

39. The method of claim 36, wherein the cell line comprises a transformed mammalian cell line.

40. The method of claim 39, wherein the cell line comprises a myeloma cell line.

41. The method of claim 36, wherein the hybridoma cell is maintained as an ascyte in a mammal.

42. The method of claim 36, wherein the antibody produced from the isolated cell is produced using recombinant biotechnology means.

43. A method of producing a molecule that that binds Bob, comprising 1) incubating SEQ ID NO:1, SEQ ID NO:2, SEQ ID NO:3, SEQ ID NO:4, SEQ ID NO:5, or SEQ ID NO:6 with a set of molecules forming a mixture, 2) isolating the molecules that bind SEQ ID NO:1, SEQ ID NO:2, SEQ ID NO:3, SEQ ID NO:4, SEQ ID NO:5, or SEQ ID NO:6 forming a set of isolated molecules, and synthesizing at least one of the isolated molecules.

44. The method of claim 43, wherein the step of isolating comprises incubating the mixture with either Bob 37 or Bob 39.

45. A method of culturing HIV comprising first culturing HIV in an amplification medium, and then culturing the HIV in an assay medium wherein the assay medium does not elicit a calcium flux.

46. The method of claim 45, further comprising infecting cells with the HIV grown in the assay medium producing infected cells.

47. The method of claim 46, wherein the cells comprise Ghost (3) cells, human osteosarcoma cells, chinese hamster ovary (CHO) cells, HEK293 cells, Jurkat cells, HT-29 cells, HCT116 cells, DLD1 cells, intestinal cells, human lymphocytes, macrophages, peripheral blood mononuclear cells, renal tubular cell lines, or Daudi cells, and wherein the cells express Bob.

48. The method of claim 45, further comprising assaying the infected cells for calcium flux.

49. The method of claim 48, wherein assaying the infected cells for calcium flux comprises assaying increased cytosolic calcium content resulting from gp120-induced, Bob-mediated activation.

52. The method of claim 45, further comprising incubating the infected cells with a potential inhibitor of Bob-gp120 binding.

53. The method of claim 45, wherein the amplification medium induces calcium flux.

54. The method of claim 53, wherein the amplification medium comprises serum.

55. The method of claim 54, wherein the serum is bovine serum.

56. The method of claim 53, wherein the amplification medium comprises phytohemaglutinin.

57. The method of claim 56, wherein the amplification medium further comprises bovine serum.

58. The method of claim 45, wherein the HIV is cultured in the amplification medium for at least 3 days.

59. The method of claim 58, wherein the amplification medium comprises RPMI 1640 with 10% fetal calf serum and phytohemaglutinin.

60. The method of claim 45, wherein the assay medium does not comprise bovine serum or phytohemaglutinin.

61. The method of claim 60, wherein the assay medium comprises AIM-V.

62. The method of claim 61, wherein the assay medium comprises 20 u/ml IL-2.

63. A method of reducing an interaction between Bob and gp120 comprising administering the composition of claim 6.

64. A method of reducing activation of lymphocytes by gp120 comprising administering the composition of claim 6.

65. A method of reducing the symptoms of HIV enteropathy, HIV nephropathy, HIV-related hyperlipidemia, or HIV-related infertility comprising administering the composition of claim 6.

66. A method of reducing HIV infection comprising reducing an interaction between gp120 and a protein expressed in a lymphocyte, wherein the interaction between gp120 and the protein occurs at less than or equal to 150 nM gp120.

67. A composition that binds Bob, wherein the composition prevents an interaction between Bob and gp120.

68. The composition of claim 67, wherein the gp120 comprises a V3 loop, and wherein the interaction between Bob and gp120 comprises an interaction between Bob and the V3 loop.

69. The composition of claim 68, wherein the V3 loop comprises SEQ ID NO: 15, SEQ ID NO:16, or SEQ ID NO:17, or a conserved variant thereof.

70. The composition of claim 68, wherein the V3 loop comprises the sequence GPG.

71. The composition of claim 2, wherein the Bob inhibitor is not a myelin binding inhibitor.

72. A composition that inhibits gp120 induced activation of HT-29 cells, wherein the composition is a polyclonal antibody that interacts with SEQ ID NO:1-3.

73. The composition of claim 6, wherein the composition lessens the amount of productive infection of CD4 lymphocytes and/or macrophages.

74. The composition of claim 73, wherein the composition delays th productive infection of CD4 lymphocytes and/or macrophages.

74. The method of claim 46, wherein the cells comprise Ghost (3) cells, human osteosarcoma cells, chinese hamster ovary (CHO) cells, HEK293 cells, or Jurkat cells.
